SEQUENCE LISTING (24 sequences)

SEQ ID NO: 1 - protein, 182 aa, Synthetic construct [sequence omitted]

SEQ ID NO: 2 - protein, 182 aa, Synthetic construct [sequence omitted]

SEQ ID NO: 3 - protein, 10 aa, Synthetic construct
KLVVVGACGV

SEQ ID NO: 4 - protein, 9 aa, Synthetic construct
CYVKDLSRL

SEQ ID NO: 5 - protein, 9 aa, Synthetic construct
RYRPDMPCF

SEQ ID NO: 6 - protein, 9 aa, Synthetic construct
CYIKILHQL

SEQ ID NO: 7 - DNA, 20 bp, Synthetic construct
  source 1..20: mol_type = other DNA
cggctactacaaccagagcg

SEQ ID NO: 8 - DNA, 20 bp, Synthetic construct
  source 1..20: mol_type = other DNA
agatcacactgacctggcag

SEQ ID NO: 9 - DNA, 20 bp, Synthetic construct
  source 1..20: mol_type = other DNA
aggtcagtgtgatctccgca

SEQ ID NO: 10 - protein, 10 aa, Synthetic construct
KLVVVGADGV

SEQ ID NO: 11 - protein, 9 aa, Synthetic construct
NLVPMVATV

SEQ ID NO: 12 - protein, 9 aa, Synthetic construct
CLVPMVATV

SEQ ID NO: 13 - protein, 9 aa, Synthetic construct
NCVPMVATV

SEQ ID NO: 14 - protein, 9 aa, Synthetic construct
NLCPMVATV

SEQ ID NO: 15 - protein, 9 aa, Synthetic construct
NLVCMVATV

SEQ ID NO: 16 - protein, 9 aa, Synthetic construct
NLVPCVATV

SEQ ID NO: 17 - protein, 9 aa, Synthetic construct
NLVPMCATV

SEQ ID NO: 18 - protein, 9 aa, Synthetic construct
NLVPMVCTV

SEQ ID NO: 19 - protein, 9 aa, Synthetic construct
NLVPMVACV

SEQ ID NO: 20 - protein, 9 aa, Synthetic construct
NLVPMVATC

SEQ ID NO: 21 - protein, 10 aa, Synthetic construct
ACDPHSGHFV

SEQ ID NO: 22 - protein, 9 aa, Synthetic construct
NLNRCSVPV

SEQ ID NO: 23 - protein, 492 aa, Synthetic construct
MATGSRTSLLLAFGLLCLPWLQEGSAKLVVVGACGVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSGDYKDDDDK

SEQ ID NO: 24 - protein, 491 aa, Synthetic construct
MATGSRTSLLLAFGLLCLPWLQEGSANLVPMVATVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSGDYKDDDDK

(19) United States
(12) Patent Application Publication
(10) Pub. No.: US 2026/0072029 A1
(43) Pub. Date: Mar. 12, 2026

(54) COMPOSITIONS AND METHODS FOR SCREENING AND IDENTIFYING IMMUNE MODULATORS

(71) Applicant: Northwestern University, Evanston, IL (US)

(72) Inventors: Xiaoyu ZHANG, Evanston, IL (US); Chenlu ZHANG, Evanston, IL (US); Chen ZHOU, Evanston, IL (US)

(21) Appl. No.: 19/083,185

(22) Filed: Mar. 18, 2025

Related U.S. Application Data
(60) Provisional application No. 63/568,719, filed on Mar. 22, 2024.

(51) Int. Cl. G01N 33/569; G01N 21/64; G01N 33/542; G01N 33/68

(52) U.S. Cl. CPC G01N 33/56977; G01N 21/6428; G01N 33/542; G01N 33/6872; G01N 2021/6439; G01N 2333/70539

(57) ABSTRACT

Provided herein are compositions, systems, methods, and kits related to chemical immunology for use in assessing and monitoring disease stages and phases, predicting the likelihood of disease progression, predicting and monitoring responses to disease therapies, and treating disease conditions.

### CROSS REFERENCE TO RELATED APPLICATIONS

This application claims priority to and the benefit of U.S. Provisional Patent Application No. 63/568,719, filed Mar. 22, 2024, which is incorporated herein by reference in its entirety and for all purposes.

### SEQUENCE LISTING PARAGRAPH

The text of the computer readable sequence listing filed herewith, titled “NWEST_43010_202_SequenceListing.xml”, created Aug. 14, 2025, having a file size of 23,171 bytes, is hereby incorporated by reference in its entirety.

### FIELD

Provided herein are compositions, systems, methods, and kits related to chemical immunology for use in assessing and monitoring disease stages and phases, predicting the likelihood of disease progression, predicting and monitoring responses to disease therapies, and treating disease conditions.

### BACKGROUND

The MHC-I antigen presentation pathways play central roles in regulating immune responses and influencing a wide array of physiological functions and disease progression. These roles hinge on the interaction between the T-cell receptor (TCR) of cytotoxic CD8+ T cells and the pMHC-I complex displayed on the cell surface. Exploring the antigen sequences presented by MHC-I is necessary for understanding how pMHC-I influences the immune system and for devising strategies for therapeutic intervention. However, due to the highly polymorphic nature of MHC-I, identifying the antigens associated with MHC-I molecules presents a challenge. Thus, improved tools for predicting antigen-MHC-I binding are needed.

### SUMMARY

The present disclosure provides compositions and methods for determining the reactivity of cysteines within the immunopeptidome. The compositions and methods advance the development and screening of therapeutic agents that target the MHC-I immunopeptidome to treat related disorders such as, for example, cancer.

Described herein is a platform for mapping reactive cysteines on MHC-I-bound peptide antigens. The probes and screening methods described herein advance understanding of reactive cysteines in immunopeptidome and contribute to the development of therapeutic agents that modulate immune function in a variety of disorders.

For example, in some embodiments, provided herein is a cell impermeable probe comprising a cysteine-reactive group linked to desthiobiotin by a linker comprising a carboxyl and/or sulfonate functional group to impart cell impermeability. In some embodiments, the cysteine-reactive group is selected from iodoacetamide, chloroacetamide, and maleimide. In some embodiments, the cysteine-reactive group is maleimide. In some embodiments, the linker comprises a sulfonate functional group to impart cell impermeability. In some embodiments, the maleimide is hydrolyzed or non-hydrolyzed.

In some embodiments, the probe has the structure:

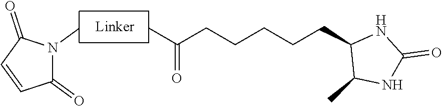

In some embodiments, the linker comprises 1-100 atoms (e.g., 1, 2, 5, 10, 20, 30, 40, 50, 60, 70, 80, 90, 100, or ranges or lengths therebetween).

In some embodiments, the linker comprises any suitable combination of CH₂, C═O, NH, —SO₃H, —COOH, —PO₄H₂, —O—, cycloalkyl rings, aryl rings, heteroalkyl rings, and heteroaryl rings.

In some embodiments, the linker comprises:

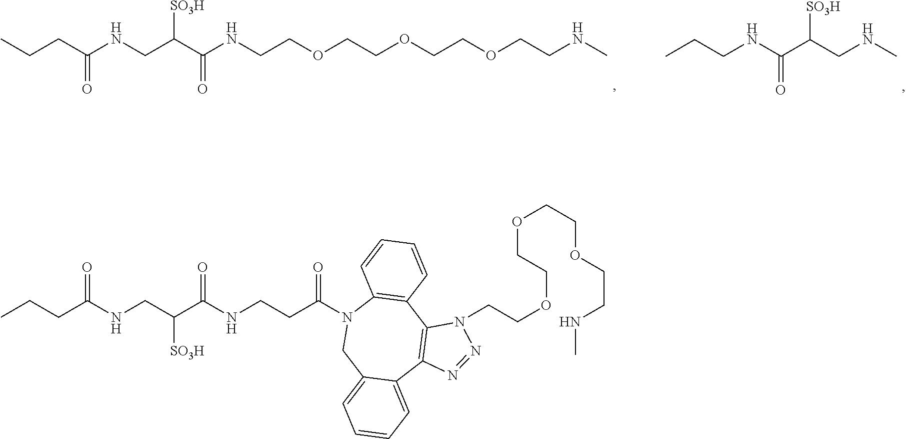

In some embodiments, the probe has the structure:

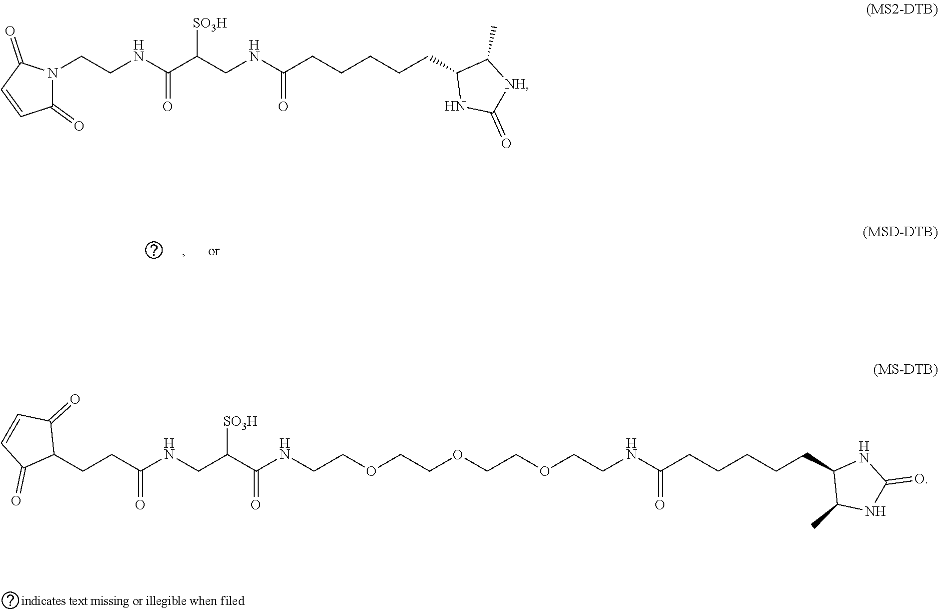 [SMILES: C[C@@H]1NC(=O)N[C@@H]1CCCCCC(=O)NCC(C(=O)NCCN1C(=O)C=CC1=O)S(=O)(=O)O ; C[C@@H]1NC(=O)N[C@@H]1CCCCCC(=O)NCCOCCOCCOCCNC(=O)C(CNC(=O)CCC1C(=O)C=CC1=O)S(=O)(=O)O]

Embodiments of the present disclosure also include a composition, kit, or system comprising a probe described herein and optionally a streptavidin-bound detectable label. In some embodiments, the detectable label is a fluorophore (e.g., fluorescein isothiocyanate (FITC)). In some embodiments, the composition, kit, or system further comprises one or more additional components selected from, for example, buffer, a cytokine (e.g., gamma interferon), a test compound, and a detection reagent.

Embodiments of the present disclosure also include a method of detecting MHC-I-associated peptides comprising contacting a sample with a probe described herein under conditions in which the cysteine-reactive group binds to extracellularly-displayed cysteine-containing peptides.

Additional embodiments provide a method of detecting MHC-I-associated peptides, comprising: a) contacting a sample comprising MHC-I with a probe described herein under conditions such that the cysteine-reactive group of the probe binds to extracellularly-displayed cysteine-containing peptides bound to MHC-1; and b) detecting peptides bound to the cysteine-reactive group. In some embodiments, the detecting comprises detecting comprises contacting the sample with a streptavidin-bound detectable label that binds to desthiobiotin. In some embodiments, the method further comprises detecting the detectable label and thereby detecting the MHC-I-associated peptides. In some embodiments, the method further comprises isolating the MHC-I-associated peptides and/or cells displaying the MHC-I-associated peptides. In some embodiments, the method further comprises identifying the MHC-I-associated peptides (e.g., by chemical proteomics). In some embodiments, the method further comprises analyzing the MHC-I-associated peptides by assay; and identifying the MHC-I-associated peptides by chemical proteomics. In some embodiments, peptides are displayed on single-chain trimers (SCTs) comprising covalently linked single chains of MHC-I, β2-microglobulin, and cysteine-containing peptides.

In some embodiments, methods of screening compounds are provided. For example, in some embodiments, the method comprises contacting the sample with a test compound; and assaying the effect of the test compound on binding of the probe to cysteines on the peptide. The present disclosure is not limited to particular test compounds. Examples include but are not limited to, immune modulators, a bispecific T cell engager (BiTEs), or a compound that induces antibody-dependent cellular phagocytosis (ADCP) (e.g., maleimide moiety bound to an Fc-binding cyclic peptide, for example,

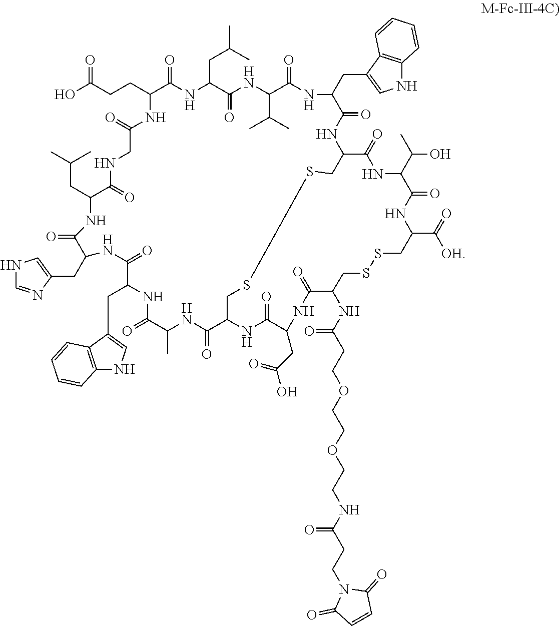 [SMILES: CC(C)CC1NC(=O)C(Cc2c[nH]cn2)NC(=O)C(Cc2c[nH]c3ccccc23)NC(=O)C(C)NC(=O)C2CSSCC(NC(=O)C(Cc3c[nH]c4ccccc34)NC(=O)C(C(C)C)NC(=O)C(CC(C)C)NC(=O)C(CCC(=O)O)NC(=O)CNC1=O)C(=O)NC(C(C)O)C(=O)NC(C(=O)O)CSSCC(NC(=O)CCOCCOCCNC(=O)CCN1C(=O)C=CC1=O)C(=O)NC(CC(=O)O)C(=O)N2]

In some embodiments, the sample is a biological sample, for example, amniotic fluid, ascites, bile, breast milk, breast milk colostrum, bronchoalveolar lavage fluid, cerebrospinal fluid, dialysate, eye aqueous humor, eye vitreous humor, feces, paracentesis, pericardial fluid, peritoneal, blood plasma, pleural, semen, blood serum, synovial fluid, tears, thoracentesis, blood, saliva, gargle, or urine.

In some embodiments, the method further comprises blocking the proximity between MHC-I and the probe and measuring how a small molecule, a biomolecule, and/or a physiological condition affects cysteine modification.

Additional embodiments are described herein.

### BRIEF DESCRIPTION OF THE DRAWINGS

FIGS. 1A-1F show development of chemical probes for mapping cysteines within the immunopeptidome. FIG. 1A shows the structures of 6 probes, with each one incorporating one of three cysteine-reactive groups (iodoacetamide, chloroacetamide, and maleimide), all linked to desthiobiotin. FIG. 1B shows MSD-DTB and MS-DTB probes exhibited greater cell surface labeling compared to the others in BV173 and MT2 cells. The result is a representative of three experiments (n=3 biologically independent experiments). FIG. 1C shows a LC-MS analysis revealing that MSD-DTB and MS-DTB probes selectively modify cysteines, but not histidine, serine, and N-terminal amine. The result is a representative of two experiments (n=2 biologically independent experiments). FIG. 1D shows the structures of MD, MSD, and MSD4, all featuring a dibenzocyclooctyne group for the azide-alkyne cycloaddition with a fluorophore. FIG. 1E shows in-gel fluorescence analyses which revealed that sulfonated maleimide probes minimally labeled proteins in live cells, while yielding comparable levels of proteome labeling to its cell-permeable counterpart probe MD in cell lysates. The result is a representative of two experiments (n=2 biologically independent experiments). FIG. 1F shows cysteine-directed ABPP analyses which revealed that MS-DTB only engaged 5 cysteines with >20% engagement out of 15,310 quantified cysteines in live cells, while it labeled 1730 cysteines with >20% engagement in cell lysates. Cysteine engagement data represent mean values (n=2 biologically independent experiments).

FIGS. 2A-2G show that sulfonated maleimide probes modify cysteines on MHC-I-bound antigens. FIG. 2A shows quantitative global proteomics and flow cytometry studies that confirmed the knockout (KO) of HLA class I genes in MT2 cells. Global proteomics data represent mean values (n=3 independent replicates). The result of flow cytometry analysis is a representative of two experiments (n=2 independent replicates). FIG. 2B shows flow cytometry analyses measuring Streptavidin-FITC staining on HLA wildtype (WT) and KO cells treated with the MSD-DTB or MS-DTB probe. Data represent mean values (n=2 independent replicates in MT2 and MDA-MB-231 cells treated with MSD-DTB, and MDA-MB-231 cells treated with MS-DTB) and mean values±SEM (n=3 independent replicates in BV173 cells treated with MSD-DTB and MS-DTB, and n=4 independent replicates in MT2 cells treated with MS-DTB). The statistical significance was assessed using unpaired two-tailed Student's t-tests. P values were 0.037, 0.0029, and 0.048. FIG. 2C shows quantitative global proteomics and flow cytometry studies that confirmed the complete knockout of HLA class I genes in MT2 cells. Global proteomics data represent mean values (n=3 independent replicates). P values were calculated by two-sided t test and adjusted using Benjamini-Hochberg correction for multiple comparisons. The result of flow cytometry analysis is a representative of two experiments (n=2 independent replicates). FIG. 2D shows flow cytometry analyses measuring Streptavidin-FITC staining on MT2 parental and HLA knockout cells treated with MS-DTB. Data represent mean values±SEM (n=4 independent replicates). The statistical significance was assessed using unpaired two-tailed Student's t-tests. P values were 0.0029 and 9.2×10⁻⁶. FIG. 2E shows the structure of NHS-sulfonate-biotin and flow cytometry analyses measuring Streptavidin-FITC staining on BV173 HLA wildtype and knockout cells treated with NHS-sulfonate-biotin. Data represent mean values (n=2 independent replicates). NHS, N-hydroxysuccinimide. FIG. 2F shows an ELISA assay measuring MS-DTB in the pMHC-I complex. Data represent mean values±SEM (n=3 independent replicates). The statistical significance was assessed using unpaired two-tailed Student's t-tests. P values were 8.1×10⁻⁵, 0.00028, 6.2×10⁻⁶, and 8.7×10⁻⁶. HRP, horseradish peroxidase. FIG. 2G shows an ELISA assay measuring MHC-I and β2-microglobulin (β2M) assembly. Data represent mean values±SEM (n=3 independent replicates). The statistical significance was assessed using unpaired two-tailed Student's t-tests.

FIGS. 3A-E show that the MHC-I immunopeptidome exhibits distinct cysteine reactivities. FIG. 3A shows a schematic representation of the immunopeptidomics workflow. FIG. 3B shows the number of 8-13-mer MHC-I-associated peptides in MT2 and BV173 cells treated with 50 μM of MS-DTB. The result is representative of two experiments (n=2 independent replicates). FIG. 3C shows motif analysis of all 9-mer MHC-I-bound antigens, cysteine-containing 9-mer MHC-I-bound antigens, cysteinylated 9-mer MHC-I-bound antigens, and MS-DTB-modified 9-mer MHC-I-bound antigens. FIG. 3D shows distribution of all cysteines, cysteinylated cysteines, and MS-DTB-modified cysteines on 9-mer peptide antigens in MT2 and BV173 cells. FIG. 3E shows a Venn diagram of MHC-I-bound antigens with unmodified, cysteinylated, and MS-DTB-modified cysteines.

FIGS. 4A-G shows the use of sulfonated maleimide probes to assess changes in cysteine reactivity of MHC-I bound antigens upon IFNγ stimulation. FIG. 4A shows a schematic representation of comparative analysis in interferon gamma (IFNγ)-stimulated versus non-stimulated BV173 cells. FIG. 4B shows Western blot analysis of MHC-I and PSMB9 expression in BV173 cells stimulated with IFNγ (50 ng/mL, 24 hours). The result is a representative of two experiments (n=2 independent replicates). FIG. 4C shows an ELISA assay measuring the presence of MS-DTB in the pMHC-I complex with or without IFNγ stimulation in BV173 cells. Data represent mean values±SEM (n=3 independent replicates). The statistical significance was evaluated through unpaired two-tailed Student's t-tests. P value was 0.025. FIG. 4D shows the number of 8-13-mer MHC-I-associated peptides in IFNγ-stimulated versus non-stimulated cells. FIG. 4E shows motif analysis of all 9-mer MHC-I-bound antigens and MS-DTB-modified MHC-I-bound antigens. FIG. 4F shows distribution of MS-DTB-modified cysteines on 9-mer MHC-I-bound antigens in IFNγ-stimulated versus non-stimulated cells. FIG. 4G shows a heatmap showing the ratio values of amino acid (aa) frequency in IFNγ-stimulated versus non-stimulated cells.

FIGS. 5A-F shows mapping of reactive cysteines on MHC-I-bound antigens using the single-chain trimer model. FIG. 5A shows a schematic representation of a native pMHC-I complex and a single-chain trimer. FIG. 5B shows constructs of SCTs of HLA-A*02:01 presenting the KRAS-G12C and KRAS-G12D neoantigens. SCT, single-chain trimer. LTR, long-terminal repeats. CMV, cytomegalovirus. SP, signal peptide. FIG. 5C shows in-gel fluorescence analysis of KRAS-G12C-SCT and KRAS-G12D-SCT from HEK293T cells treated with the MSD probe (5 μM, 30 minutes). The result is representative of three experiments (n=3 independent replicates) TAMRA, tetramethylrhodamine. FIG. 5D shows an ELISA assay measuring the assembly of MHC-I and β2-microglobulin in the presence of sulfonated maleimide probes. Data represent mean values±SEM (n=3 independent replicates). FIG. 5E shows modeling studies that indicate that the cysteine in the KRAS-G12C neoantigen is exposed to the solvent when bound to MHC-I. FIG. 5F shows in-gel fluorescence analysis of SCTs of HLA-A*02:01 presenting pp65 antigens with cysteines introduced at positions 1-9 from HEK293T cells treated with the MSD probe (50 μM, 30 minutes). The bar graph represents quantification of the TAMRA/FLAG ratio values following FLAG immunoprecipitation (IP). Data represent mean values (n=2 independent replicates).

FIG. 6A-D shows reactivity-based antigen profiling for the discovery of reactive cysteines on MHC-I-bound antigens. FIG. 6A shows a schematic representation of the reactivity-based antigen profiling workflow. FIG. 6B shows a Violin plot comparing the predicted MHC-I binding ranks of MS-DTB-enriched 8-10-mer peptides in MT2 parental and HLA knockout cells. The statistical significance was evaluated through unpaired two-tailed Student's t-tests. P values were 1.2×10⁻⁵ and 6.2×10⁻⁸. The result is representative of two experiments (n=2 independent replicates). FIG. 6C shows motif analysis of MS-DTB-modified 9-mer peptides identified in MT2 parental cells. The result is representative of two experiments (n=2 independent replicates). FIG. 6D shows validation of MS-DTB-modified peptides using SCTs. The result is representative of two experiments (n=2 independent replicates). FIG. 6E shows modeling studies that indicate that cysteines in the MS-DTB-modified peptides are solvent-exposed when forming pMHC-I complexes.

FIGS. 7A-7E show targeting of reactive cysteines on MHC-I-bound antigens to induce ADCP. FIG. 7A shows the structure of M-Fc-III-4C. FIG. 7B shows a schematic representation of anti-CD20-induced antibody-dependent cellular phagocytosis (ADCP) between Jurkat ADCP reporter cells and Raji cells. IgG, immunoglobulin G. FIG. 7C shows an ADCP assay measuring luciferase activation in Jurkat-Luc NFAT-CD32 cells after co-culturing with Raji cells in the presence of anti-CD20 antibody. Data represent mean values±SEM (n=3 independent replicates). The statistical significance was evaluated through unpaired two-tailed Student's t-tests. P values were 0.000078 and 0.000075. FIG. 7D shows a schematic representation of targeting reactive cysteines on MHC-I-bound antigens to induce ADCP. FIG. 7E shows an ADCP assay measuring luciferase activation in Jurkat-Luc NFAT-CD32 cells after co-culturing with M-Fc-III-4C-treated MT2 parental and HLA knockout cells in the presence of human IgG. Data represent mean values±SEM (n=3 independent replicates). The statistical significance was evaluated through unpaired two-tailed Student's t-tests. P values were 0.0066 and 0.00027.

FIG. 8 shows gating strategies for flow cytometry analysis in FIG. 1B.

FIGS. 9A-F show development of chemical probes for mapping reactive cysteines within the immunopeptidome. FIG. 9A shows MS1 spectra of MS-DTB-peptide and MSD-DTB-peptide adducts from the LC-MS analysis in FIG. 1C. FIG. 9B shows in-gel fluorescence analysis demonstrating that the MSD probe selectively labels cysteine-containing peptide. The result is representative of two experiments (n=2 independent replicates). FIG. 9C shows assessment of cytotoxicity for MD, MSD, MSD-DTB, and MS-DTB probes in HEK293T cells after 72 hours of treatment. Data represent mean values±SEM (n=3 independent replicates). FIG. 9D shows a schematic representation of employing MSD-DTB and MS-DTB in proteomics workflow to identify probe-modified cysteine-containing peptides. FIG. 9E shows the number of cysteines modified by IA-DTB, MSD-DTB and MS-DTB. The result is representative of two experiments (n=2 independent replicates). FIG. 9F shows MS1 spectra of two representative peptides modified by MS-DTB in the orbitrap mass analyzer.

FIGS. 10A-H shows that sulfonated maleimide probes modify cysteines on MHC-I-bound antigens. FIG. 10A shows that quantitative global proteomics and flow cytometry studies confirmed the knockout of HLA class I genes in MDA-MB-231 cells. Global proteomics data represent mean values (n=3 independent replicates). P values were calculated by two-sided t test and adjusted using Benjamini-Hochberg correction for multiple comparisons. LFC, log 2 fold change. The result of flow cytometry analysis is a representative of two experiments (n=2 independent replicates). FIG. 10B shows that quantitative global proteomics and flow cytometry studies confirmed the knockout of HLA class I genes in BV173 cells. Global proteomics data represent mean values (n=3 independent replicates). P values were calculated by two-sided t test and adjusted using Benjamini-Hochberg correction for multiple comparisons. The result of flow cytometry analysis is a representative of two experiments (n=2 independent replicates). FIG. 10C shows gating strategies for flow cytometry analysis in FIG. 9A. FIG. 10D shows gating strategies for flow cytometry analysis in FIG. 9B. FIG. 10F shows bar graph quantification from global proteomics indicates the knockout of HLA class I genes, but not HLA class II genes. Data represent mean values (n=2 independent replicates for comparing HLA WT to partial KO, n=3 independent replicates for comparing HLA WT to complete KO). FIG. 10G shows an ELISA assay measuring the MS-DTB probe labeling on pMHC-I complex at pH of 5-8 in BV173 cells. Data represent mean values±SEM (n=3 independent replicates). The statistical significance was assessed using unpaired two-tailed Student's t-tests. P values were 0.0034, 0.0059, and 0.00082. FIG. 10G shows an ELISA assay measuring the MS-DTB probe labeling on pMHC-I complex in BV173 cells pretreated with H₂O₂. Data represent mean values±SEM (n=3 independent replicates). The statistical significance was assessed using unpaired two-tailed Student's t-tests. P values were 0.011, 0.0022, and 0.00011. FIG. 10H shows mRNA expression of genes encoding for MHC-I and MHC-II in BV173 and MDA-MB-231 cells.

FIGS. 11A-E show gating strategies for flow cytometry analysis in FIG. 2. FIG. 11A shows gating strategies for flow cytometry analysis in FIG. 2A. FIG. 11B shows gating strategies for flow cytometry analysis in FIG. 2B. FIG. 11C shows gating strategies for flow cytometry analysis in FIG. 2C. FIG. 11D shows gating strategies for flow cytometry analysis in FIG. 2D. FIG. 11E shows gating strategies for flow cytometry analysis in FIG. 2E.

FIGS. 12A-J show MHC-I immunopeptidomics in MT2 and BV173 cells treated with MS-DTB. FIG. 12A shows MS1 base peak chromatograms of immunopeptidomics comparing wildtype versus HLA knockout in MT2 and BV173 cells. FIG. 12B shows MS1 spectra of four major peaks identified in BV173 HLA knockout cells. FIG. 12C shows the number of 8-13-mer MHC-I-associated peptides in MT2 and BV173 cells. The result is representative of two experiments (n=2 independent replicates). FIG. 12D shows distribution motif of 8-mer, 9-mer and 10-mer antigens associated with HLA-A*24:02, HLA-B*40:01 (MT2) and HLA-A*02:01, HLA-B*18:01 (BV173), and motif analysis of all 8-mer and 10-mer MHC-I-bound antigens identified by immunopeptidomics in this study. The result is representative of two experiments (n=2 independent replicates). FIG. 12E shows the number of 8-13-mer MHC-I-associated peptides in untreated MT2 and BV173 cells. The result is representative of two experiments (n=2 independent replicates). FIG. 12F shows the percentage of all, unmodified and cysteinylated 8-13-mer antigens in untreated and MS-DTB-treated MT2 and BV173 cells. The result is representative of two experiments (n=2 independent replicates). FIG. 12G shows a Venn diagram of 8-mer, 9-mer and 10-mer MHC-I-associated peptides with unmodified, cysteinylated, and MS-DTB-modified cysteines. The result is representative of two experiments (n=2 independent replicates). FIG. 12H shows distribution of all cysteines, cysteinylated cysteines, and MS-DTB-modified cysteines on 8-mer and 10-mer MHC-I-associated peptides in MT2 and BV173 cells. The result is representative of two experiments (n=2 independent replicates). FIG. 12I shows the number of 8-13-mer MHC-I-associated cysteine-containing peptides and the percentage of each type of cysteine modification in MT2 and BV173 cells. The result is representative of two experiments (n=2 independent replicates). FIG. 12J shows overlap analyses comparing unmodified cysteines in untreated cells with MS-DTB-modified or unmodified cysteines in MS-DTB-treated cells. The result is representative of two experiments (n=2 independent replicates).

FIGS. 13A-D shows analysis of MS-DTB-modified cysteines associated with various HLA alleles. FIG. 13A shows distribution motif of 9-mer antigens associated with HLA-A*02:01, HLA-A*30:01, HLA-B*15:10, HLA-B*18:01, HLA-C*03:04, HLA-C*12:03, HLA-A*24:02 and HLA-B*40:01. FIG. 13B shows motif analysis of MS-DTB-modified 9-mer MHC-I-bound peptides identified by immunopeptidomics in this study. The result is representative of two experiments (n=2 independent replicates). FIG. 13D shows distribution of MS-DTB-modified cysteines on 9-mer MHC-I-bound peptides associated with HLA-A*02:01, HLA-B*15:10 and HLA-B*18:01. The result is representative of two experiments (n=2 independent replicates). FIG. 13D shows distribution of MS-DTB-modified cysteines on 9-mer MHC-I-bound peptides associated with HLA-A*24:01 and HLA-B*40:01. The result is representative of two experiments (n=2 independent replicates).

FIG. 14 shows translated single-chain peptide sequence from the SCT construct of HLA-A2/KRAS-G12C and HLA-A2/pp65.

FIGS. 15A-D shows reactivity-based profiling of reactive cysteines in the immunopeptidome. FIG. 15A shows a schematic representation of employing MS-DTB to identify the KRAS-G12C neoantigen. FIG. 15B shows MS1 peak chromatograms with extracted m/z values for MS-DTB-modified KRAS-G12C neoantigen, and the MS2 spectrum of MS-DTB-modified KRAS-G12C neoantigen. The result is representative of three experiments (n=3 independent replicates). FIG. 15C shows the number of 8-12-mer MS-DTB-modified peptides identified in MT2 wildtype and HLA knockout cells. The result is representative of two experiments (n=2 independent replicates). FIG. 15D shows distribution of MS-DTB-modified cysteines on 9-mer peptides associated with HLA-A*24:01 and HLA-B*40:01 identified through probe enrichment experiments. The result is representative of two experiments (n=2 independent replicates).

FIGS. 16A-B shows a comparative analysis of MS-DTB-modified 8-10-mer peptides in probe-pulldown experiments and immunopeptidomics. FIG. 16A shows a Venn diagram showing the overlap of MS-DTB-modified 8-10-mer antigens identified in probe-pulldown experiments and immunopeptidomics. The result is representative of two experiments (n=2 independent replicates). FIG. 16B shows a Violin plot comparing the MHC-I binding ranks of MS-DTB-modified 8-10-mer antigens identified only in probe-pulldown experiments versus those identified in both methods. The statistical significance was evaluated through unpaired two-tailed Student's t-tests.

FIG. 17 shows a comparative analysis of MS-DTB-modified 13-16-mer peptides in HLA WT and KO cells. The result is representative of two experiments (n=2 independent replicates).

FIGS. 18A-B show dose- and time-dependent labeling of DLGAP5-SCT by the sulfonated maleimide probes. FIG. 18A shows in-gel fluorescence analysis of DLGAP5-SCT (HLA-A*24:02) from HEK293T cells transfected with DLGAP5-SCT and treated with the MSD probe (1-100 μM, 30 minutes). The graph represents quantification of the DLGAP5-SCT fluorescence content. Data are presented as mean values (n=2 independent replicates). FIG. 18B shows ELISA analysis of DLGAP5-SCT (HLA-A*24:02) from HEK293T cells transfected with DLGAP5-SCT and treated with the MS-DTB probe (1-100 μM for 30 minutes, or 50 UM for 5, 10, 30, 60 and 120 minutes). Data are presented as mean values (n=2 independent replicates).

FIG. 19 shows HPLC and MS analysis of M-Fc-III-4C

FIG. 20 shows ¹H NMR and ¹³C NMR spectra of MS-DTB

FIG. 21 shows ¹H NMR and ¹³C NMR spectra of MSD-DTB

FIG. 22 shows ¹H NMR and ¹³C NMR spectra of IA-DTB-COOH.

FIG. 23 shows a synthesized shorter sulfonated maleimide probe, MS2-DTB, and an ELISA assay employed to demonstrate its ability to label reactive cysteines on MHC-I-bound antigens.

### DETAILED DESCRIPTION

Unless otherwise defined, all technical and scientific terms used herein have the same meaning as commonly understood by one of ordinary skill in the art. In case of conflict, the present document, including definitions, will control. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting. Section headings as used in this section and the entire disclosure herein are merely for organizational purposes and are not intended to be limiting.

Embodiments of present disclosure provide compositions, systems, methods, and kits related to chemical immunology for use in assessing and monitoring disease stages and phases, predicting the likelihood of disease progression, predicting and monitoring responses to disease therapies, and treating disease conditions.

In particular, experiments described herein resulted in the development of cell-impermeable sulfonated maleimide probes capable of capturing reactive cysteines within the immunopeptidome. These probes were used in chemoproteomic experiments to measure reactivity of cysteines on MHC-I-bound antigens and investigate the effect of interferon-gamma stimulation on the reactivity of cysteines. Further experiments demonstrated that targeting reactive cysteines on MHC-I-bound antigens with a maleimide-conjugated Fc-binding cyclic peptide contributes to the induction of antibody-dependent cellular phagocytosis.

The compositions and methods described herein find use in research, screening, and therapeutic applications. Exemplary compositions and methods are described herein.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures.

The singular forms “a,” “and” and “the” include plural references unless the context clearly dictates otherwise.

The present disclosure also contemplates other embodiments “comprising,” “consisting of” and “consisting essentially of,” the embodiments or elements presented herein, whether explicitly set forth or not.

Ranges can be expressed herein as from “about” one particular value, and/or to “about” another particular value. When such a range is expressed, some embodiments includes from the one particular value and/or to the other particular value. Similarly, when values are expressed as approximations, by use of the antecedent “about,” it will be understood that the particular value forms an embodiment. It will be further understood that the endpoints of each of the ranges are significant both in relation to the other endpoint, and independently of the other endpoint. It is also understood that there are a number of values disclosed herein, and that each value is also herein disclosed as “about” that particular value disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” are also disclosed.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the terms “providing,” “administering,” and “introducing,” are used interchangeably herein and refer to the placement into a cell, organism, or subject by a method or route which results in at least partial localization to a desired site. For example, the compositions disclosed herein can be administered by any appropriate route which results in delivery to a desired location in the cell, organism, or subject.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier and/or excipient. When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of proteins, nucleic acids, or compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice, and guinea pigs, and the like. Examples of non-mammals include, but are not limited to, birds, fish, and the like. In one embodiment of the methods provided herein, the mammal is a human.

A “sample” may be from human or non-human and may include, for example, amniotic fluid, ascites, bile, breast milk, breast milk colostrum, bronchoalveolar lavage fluid, cerebrospinal fluid, dialysate, eye aqueous humor, eye vitreous humor, feces, paracentesis, pericardial fluid, peritoneal, blood plasma, pleural, semen, blood serum, synovial fluid, tears, thoracentesis, blood, saliva, gargle, or urine.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

As used herein, the term “impermeable” or “impermeability” means to not permit passage (e.g., of a compound, substance, etc.) through a barrier (e.g., cell membrane). For example, “cell impermeable” means to not permit passage (e.g., of a probe) through the cell membrane. In some embodiments, a composition is cell impermeable if when a cell is contacted with the composition, above background amounts of the composition cannot be detected within the cell.

### 2. COMPOSITIONS

Embodiments of the present disclosure include a cell impermeable probe comprising a cysteine-reactive group linked to desthiobiotin by a linker comprising a carboxyl and/or sulfonate functional group to impart cell impermeability.

In some embodiments, the probe is maleimide-sulfonate-dibenzocyclooctyne-DTB (MSD-DTB), MS2-DTB, or maleimide-sulfonate-DTB (MS-DTB).

In some embodiments, the probe comprises a cysteine-reactive group (e.g., a residue-specific reagent (e.g., a reactive compound with minimal noncovalent affinity to a particular binding site (e.g., a cysteine alkylating agent (e.g., iodoacetamide or methylmethanthiosulfinate), an affinity label (e.g., a reactive compound which forms an initial noncovalent complex to a particular binding site), or a mechanism-based inhibitor)). In some embodiments, the cysteine-reactive group is maleimide. In some embodiments, the cysteine-reactive group is iodoacetamide. In some embodiments, the cysteine-reactive group is chloroacetamide.

In some embodiments, the cysteine-reactive group is linked to a biotin analogue that binds less tightly to biotin-binding proteins and is easily displaced by biotin (e.g., desthiobiotin) by a linker.

In some embodiments, the linker is a cleavable (e.g., acid cleavable (e.g., PEGylated (e.g., CL2A (e.g., PEG8 and triazole-containing linker)) or hydrazone) or enzyme cleavable (e.g., peptide-like (e.g., valine-citrulline, cyclobutene-1,1-dicarboxamide-citrulline, glycine-glycine-phenylalanine-glycine), pyrophosphate, or carbohydrate). The linker can be a noncleavable chemical compound that connects a functional molecule (e.g., a biomolecule) with a molecular tag to form a conjugate).

The linker can be used in combination with a self-immolative group (e.g., a linker which spontaneously degrades in response to a specific stimulus (e.g., para-aminobenzyl alcohol group). In some embodiments, the linker comprises a sulfonate functional group to impart cell impermeability. In some embodiments, the linker comprises a carboxyl functional group to impart cell impermeability.

The probe finds use in T cell epitope discovery, vaccine development, and immunology research (e.g., cancer immunology). For example, the probe can be used to identify tumor antigens presented on MHC-I by CD8+ T cells. The probe can be used to facilitate recognition by CD8+ T cells of antigens from pathogenic proteins on MHC-I.

In some embodiments, the probe labels extracellular cysteines in BV173 (human B cell leukemia) and MT2 (human T cell leukemia) cells.

In some embodiments, the probe has the structure:

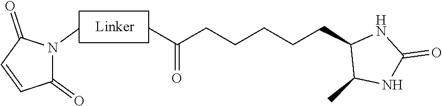

In some embodiments, the linker comprises 1-100 atoms (e.g., 1, 5, 10, 15, 20, 30, 40, 50, 60, 70, 80, 90, 100, 1-5, 1-10, 1-20, 1-30, 1-40, 1-50, 1-60, 1-70, 1-80, 1-90, 5-10, 5-20, 5-30, 5-40, 5-50, 5-60, 5-70, 5-80, 5-90, 5-100, 10-20, 10-30, 10-40, 10-50, 10-60, 10-70, 10-80, 10-90, 10-100). In some embodiments, the linker comprises 1, 5, 10, 15, 20, 30, 40, 50, 60, 70, 80, 90, 100 atoms.

In some embodiments, a linker provides sufficient distance between the cysteine reactive group and the desthiobiotin to allow each to function undisturbed (or minimally disturbed) by the linkage to the other.

In some embodiments, linkers are straight or branched chains comprising any combination of alkyl, alkenyl, or alkynyl chains, and main-chain heteroatoms (e.g., O, S, N, P, etc.). In some embodiments, linker moieties comprises one or more backbone groups selected from of: —O—, —S—, —CH—CH—, ═C═, a carbon-carbon triple bond, C—O, NH, SH, OH, CN, etc. In some embodiments, a linker moiety comprises one or more substituents, pendants, side chains, etc., comprising any suitable organic functional groups (e.g., —SO₃H OH, NH₂, CN, ═O, SH, halogen (e.g., Cl, Br, F, I), COOH, CH₃, etc.).

In some embodiments, the linker comprises any suitable combination of CH₂, C═O, NH, —SO₃H, —COOH, —PO₄H₂, —O—, cycloalkyl rings, aryl rings, heteroalkyl rings, and heteroaryl rings.

In some embodiments, the linker comprises:

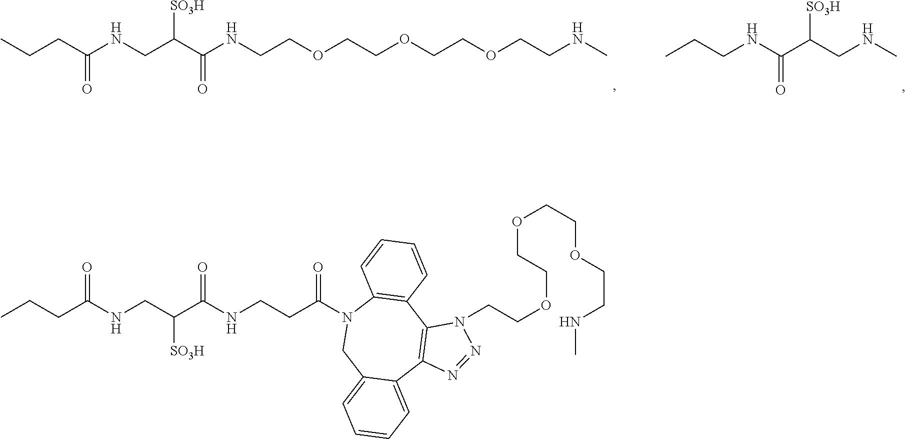

In some embodiments, the probe has the structure:

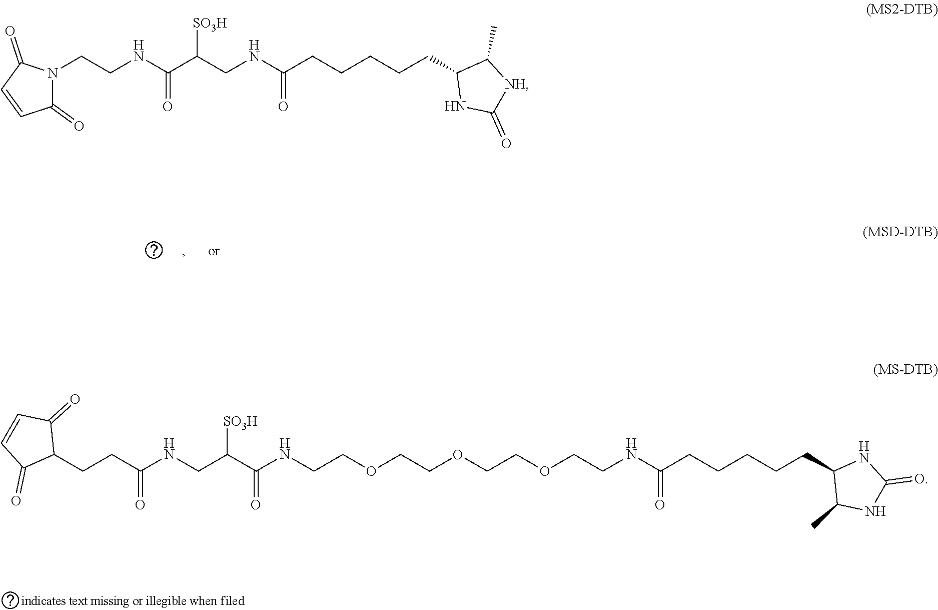 [SMILES: C[C@@H]1NC(=O)N[C@@H]1CCCCCC(=O)NCC(C(=O)NCCN1C(=O)C=CC1=O)S(=O)(=O)O ; C[C@@H]1NC(=O)N[C@@H]1CCCCCC(=O)NCCOCCOCCOCCNC(=O)C(CNC(=O)CCC1C(=O)C=CC1=O)S(=O)(=O)O]

### 3. COMPOSITION, KITS, AND SYSTEMS

Embodiments of the present disclosure also include a composition, kit, or system comprising a probe described herein and a detectable label (e.g., a functional group for binding and a tag). In some embodiments, the detectable label is a streptavidin-bound detectable label.

In some embodiment, the detectable label is a fluorophore (e.g., a fluorescent chemical compound that can re-emit light upon light excitation (e.g., an organic dye (e.g., fluorescein, rhodamine, AMCA), a biological fluorophore (e.g., green fluorescent protein, phycoerythrin, allophycocyanin), and a quantum dot)). In some embodiment, the fluorophore is fluorescein isothiocyanate (FITC). The detectable label can be a hapten molecule (e.g., biotin) or an enzyme.

The detectable label finds use, together with the probe, in qualitative and quantitative methodology. For example, enzymatic detectable labels find use in enzyme-linked immunosorbent assays (ELISA) and immunohistochemistry while fluorescent detectable labels finds use in flow cytometry, immunofluorescence, and general cellular imaging. Hapten detectable labels find use in protein isolation and purification, etc.

In some embodiments, the composition, kit, or system further comprises one or more additional components selected from, for example, buffer, a cytokine (e.g., gamma interferon), a test compound, and a detection reagent.

In some embodiments, kit or systems additionally comprise one or more of samples, control samples, containers for holding or storing a sample; one or more instruments for assisting with obtaining a test sample; reaction vessels, mixing vessels, and instructions for use of the kit or system.

### 4. METHODS

The present disclosure further provides methods of capturing and identifying MHC-I bound peptides (e.g., MHC-I antigens). The present disclosure further provides methods of detecting, assays, or screening compounds that modulate MHC-I associated peptide binding to MHC-I complexes. The methods find use in research, screening, and therapeutic applications.

For example, in some embodiments, provided herein is a method of detecting MHC-I-associated peptides, comprising: a) contacting a sample comprising MHC-I with a probe described herein under conditions such that the cysteine-reactive group of the probe binds to extracellularly-displayed cysteine-containing peptides bound to MHC-I; and b) detecting peptides bound to the cysteine-reactive group.

In some embodiments, the sample is amniotic fluid, ascites, bile, breast milk, breast milk colostrum, bronchoalveolar lavage fluid, cerebrospinal fluid, dialysate, eye aqueous humor, eye vitreous humor, feces, paracentesis, pericardial fluid, peritoneal, blood plasma, pleural, semen, blood serum, synovial fluid, tears, thoracentesis, blood, saliva, gargle, or urine.

As described herein and in the examples below, in some embodiments, the detecting comprises detecting comprises contacting the sample with a streptavidin-bound detectable label that binds to desthiobiotin.

In some embodiments, peptides are bound to native or endogenous MHC-I complexes. In such embodiments, any number of cell lines that express MHC-I complexes or are engineered to express MHC-I complexes may be utilized. Exemplary cell lines are described herein.

In some embodiments, peptides are displayed on single-chain trimers (SCTs) comprising covalently linked single chains of MHC-I, β2-microglobulin, and cysteine-containing peptides.

In some embodiments, the method further comprises identifying the MHC-I-associated peptides by chemical proteomics (e.g., the design and development of small molecule probes to understand and identify protein function. Chemical proteomics finds use in identifying the protein binding partners or targets of small molecules in cells.

In some embodiments, the method further comprises analyzing the MHC-I-associated peptides by assay and identifying the MHC-I-associated peptides by chemical proteomics. In some embodiments, the assay is flow cytometry. In some embodiments, the assay is ELISA.

In some embodiments, the method further comprises blocking the proximity between MHC-I and the probe and measuring how a small molecule, a biomolecule, and/or a physiological condition affects cysteine modification.

In some embodiments, the method further comprises using the probe to develop BiTEs for immunotherapy treatment.

In some embodiments, the method further comprises screening for small molecules or biomolecules that engage cysteine-containing peptides. The screening finds use in assessing and monitoring disease stages and phases, predicting the likelihood of disease progression, predicting and monitoring responses to disease therapies, and treating disease conditions. For example, the screening finds such use for immune system related diseases and immune disorders (e.g., asthma, ataxia telangiectasis, autoimmune polyglandular syndrome, Burkitt lymphoma, type I diabetes, DiGeorge syndrome, familial Mediterranean fever, immunodeficiency with hyper-IgM, leukemia, severe combined immunodeficiency, Crohn's disease, cancer).

For example, in some embodiments, screening methods comprise contacting the sample with a test compound; and assaying the effect of the test compound on binding of the probe to cysteines on the peptide. The present disclosure is not limited to particular test compounds. Examples include but are not limited to, immune modulators, a bispecific T cell engager (BiTEs), or a compound that induces antibody-dependent cellular phagocytosis (ADCP) (e.g., maleimide moiety bound to an Fc-binding cyclic peptide, for example,

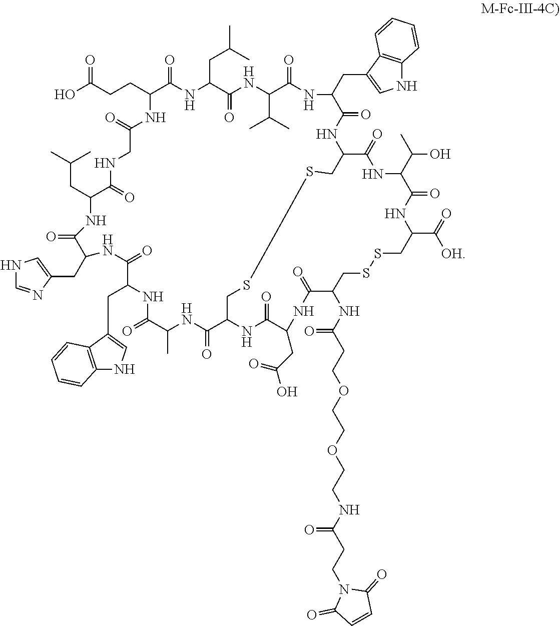 [SMILES: CC(C)CC1NC(=O)C(Cc2c[nH]cn2)NC(=O)C(Cc2c[nH]c3ccccc23)NC(=O)C(C)NC(=O)C2CSSCC(NC(=O)C(Cc3c[nH]c4ccccc34)NC(=O)C(C(C)C)NC(=O)C(CC(C)C)NC(=O)C(CCC(=O)O)NC(=O)CNC1=O)C(=O)NC(C(C)O)C(=O)NC(C(=O)O)CSSCC(NC(=O)CCOCCOCCNC(=O)CCN1C(=O)C=CC1=O)C(=O)NC(CC(=O)O)C(=O)N2]

In some embodiments, methods for assaying T-cell recruitment are provided. For example, in some embodiments, a probe of the present disclosure is conjugated to a T cell-recruiting moiety (e.g., a CD3-binding single-chain variable fragment (scFv)), and the resulting molecule is used to assay T cell recruitment.

### 5. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

### Development of Cell-Impermeable Cysteine-Reactive Probes

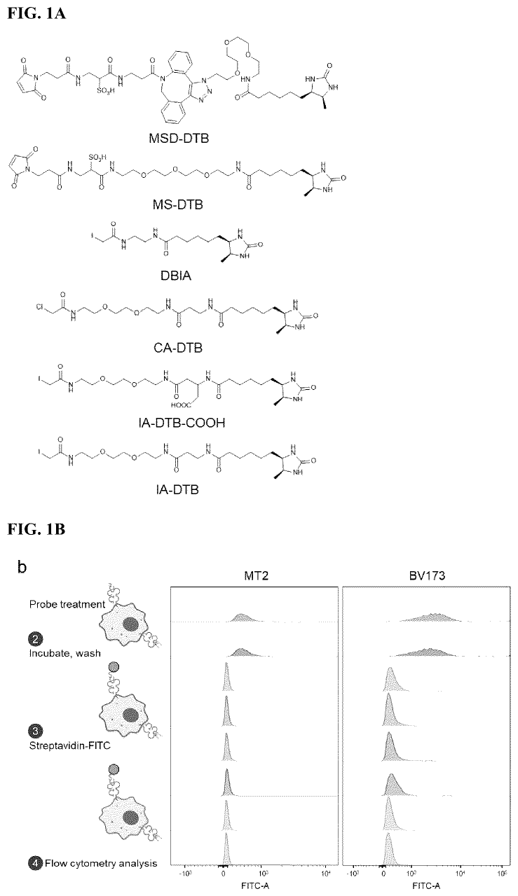

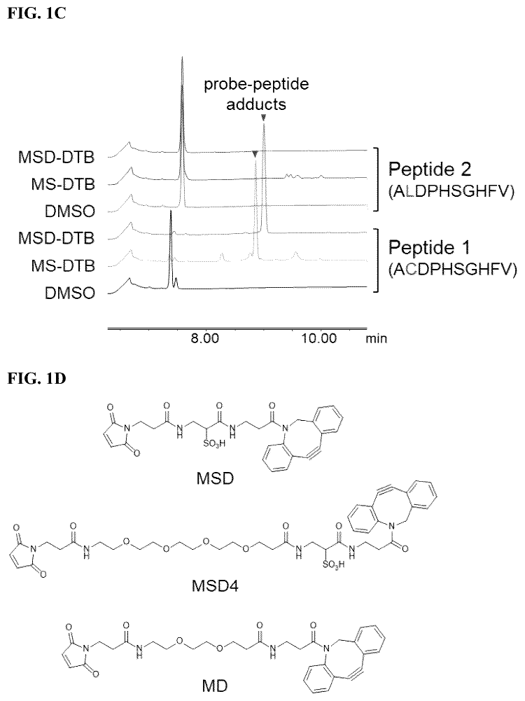

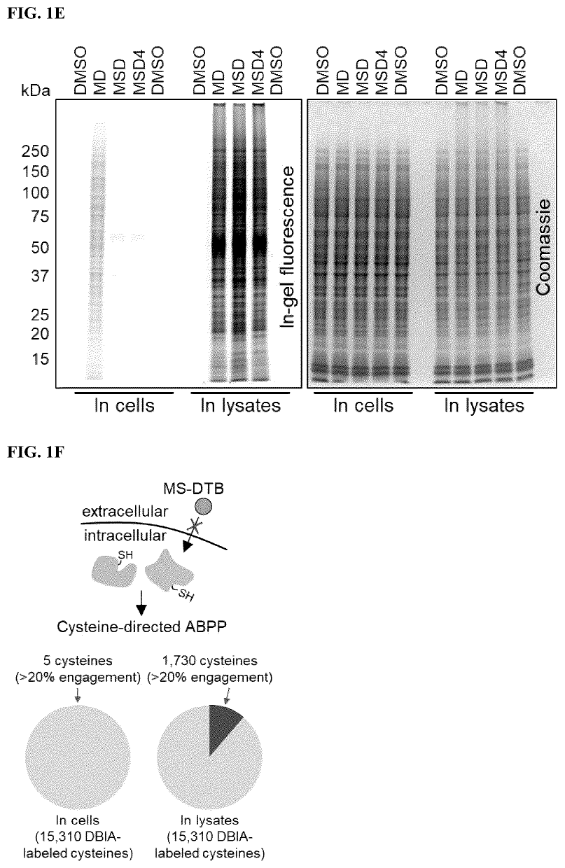

Three key features should be considered when designing broad-spectrum probes for mapping reactive cysteines within the immunopeptidome: 1) spatial specificity: The probes remain in the extracellular space. 2) residue specificity: The probes selectively target cysteines without reacting with other amino acids; and 3) quantification capability: The unique modification generated by the probe can be quantified using various analytical techniques, including flow cytometry, fluorometric assays, and mass spectrometry (MS). Due to the oxidizing environment of the extracellular milieu, which leads to interchain disulfide bond formation among extracellular protein cysteines (Yi, M. C. & Khosla, C. Thiol-Disulfide Exchange Reactions in the Mammalian Extracellular Environment. Annu Rev Chem Biomol Eng 7, 197-222 (2016)), it was contemplated that that cell-impermeable cysteine-reactive probes may primarily label cysteines within the immunopeptidome. In accordance with these criteria, six probes, each incorporating one of three cysteine-reactive groups (iodoacetamide, α-chloroacetamide, and maleimide), all linked to desthiobiotin (DTB), were synthesized (FIG. 1a). To achieve spatial specificity, a negatively charged group, such as carboxyl or sulfonate, was introduced, a strategy previously used to confer cell impermeability to small molecules (Caldwell, S. T. et al. Photoactivated release of membrane impermeant sulfonates inside cells. Chem Commun (Camb) 57, 3917-3920 (2021)).

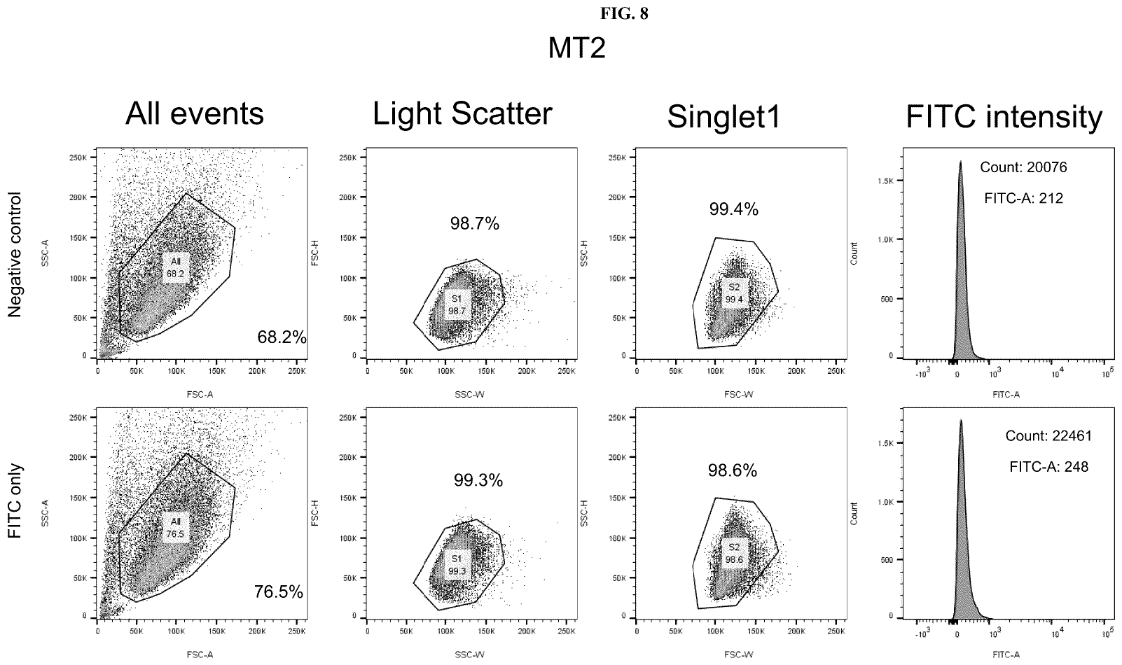

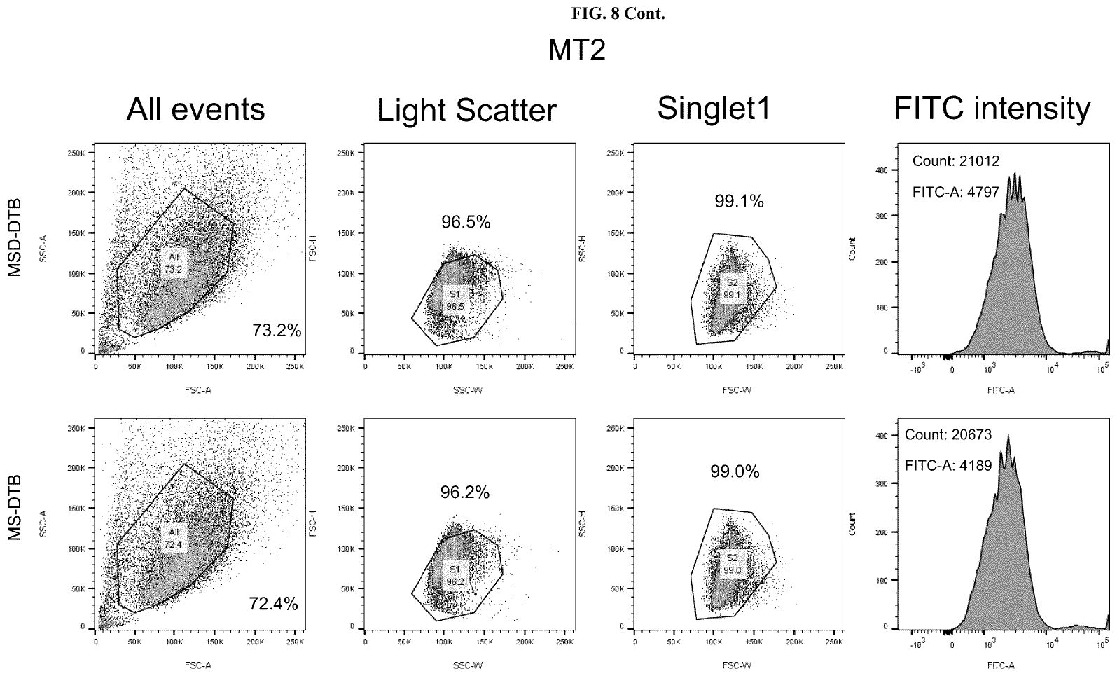

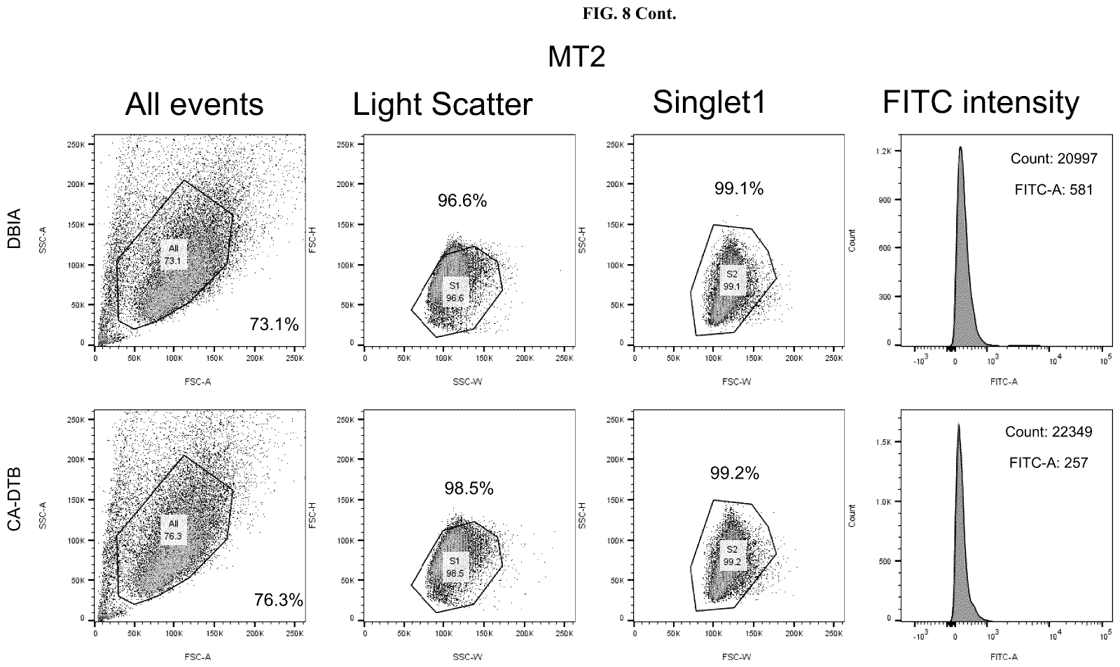

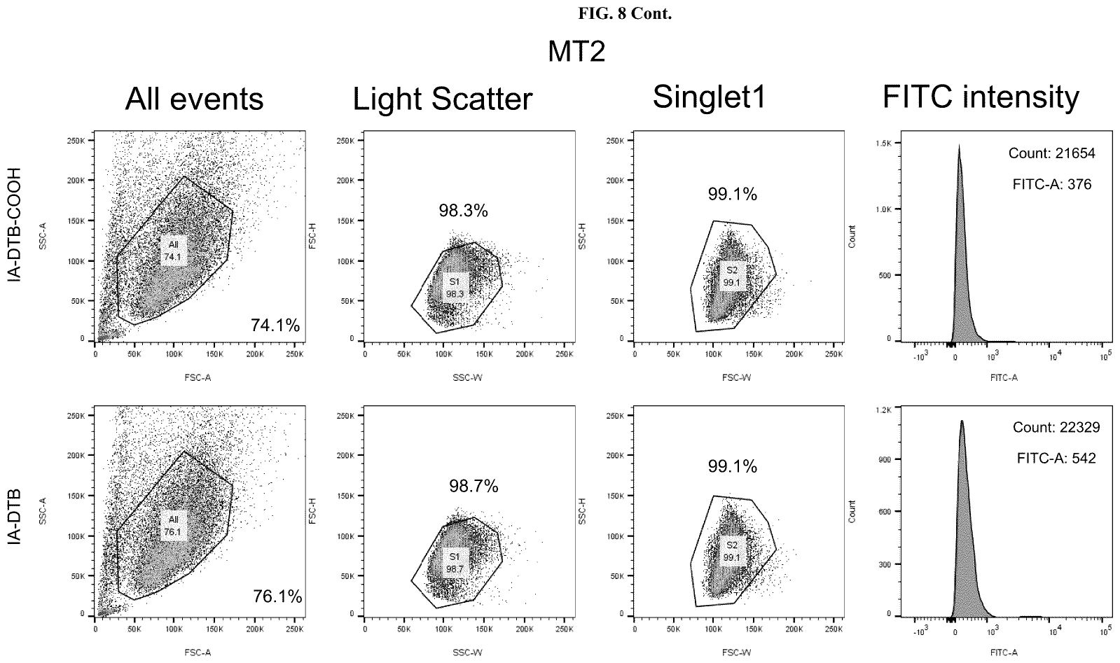

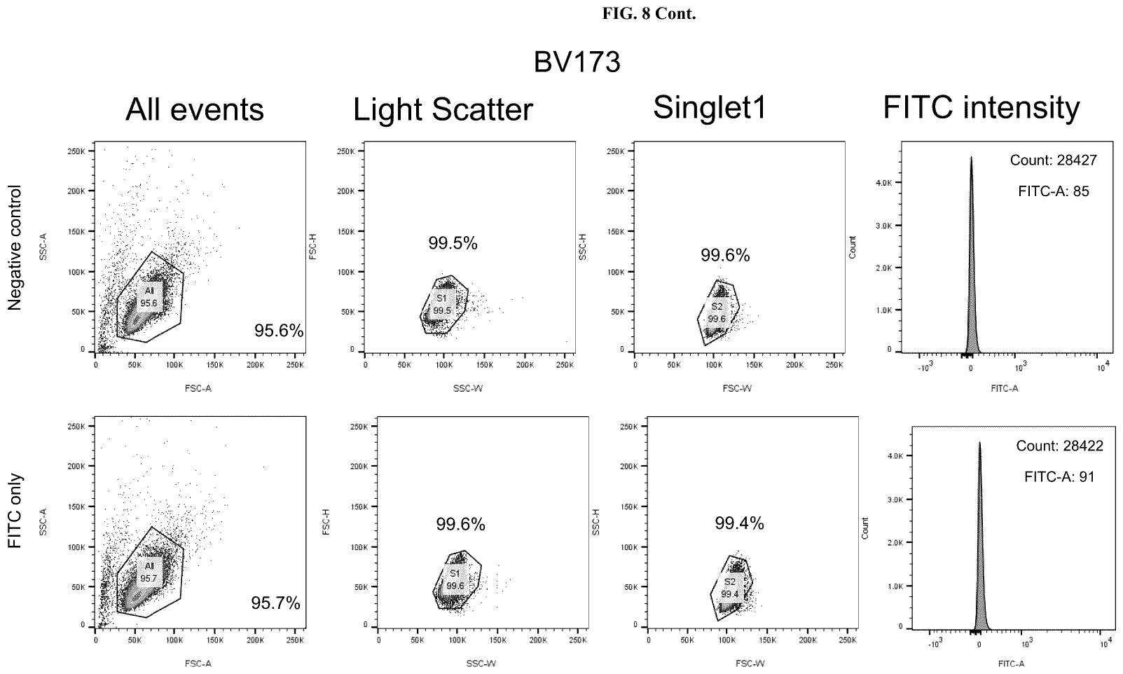

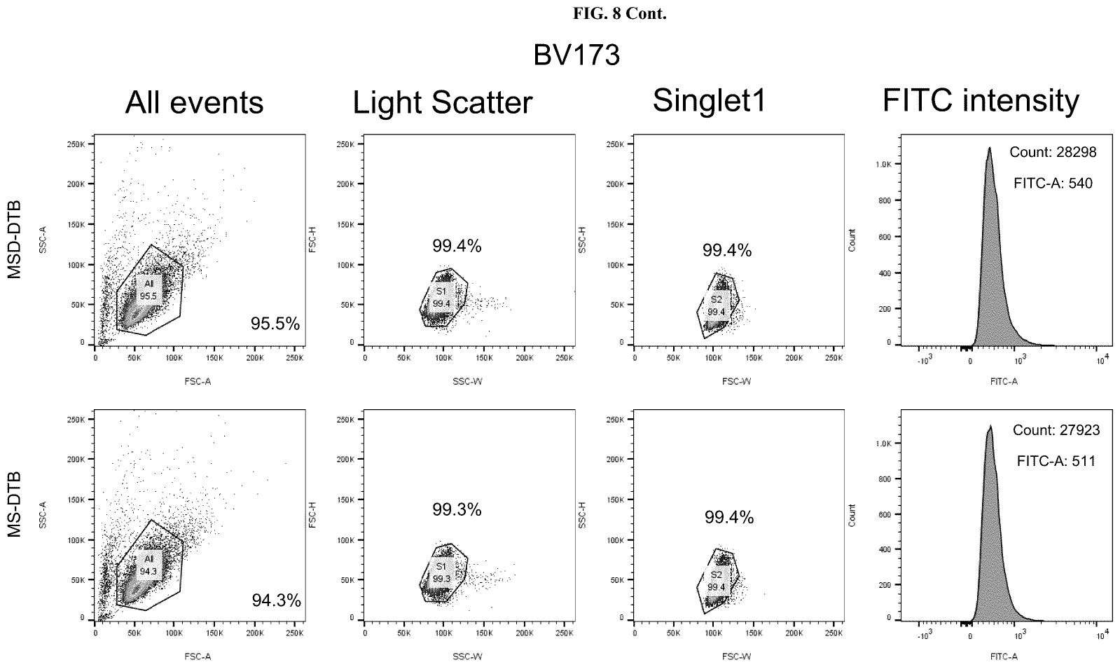

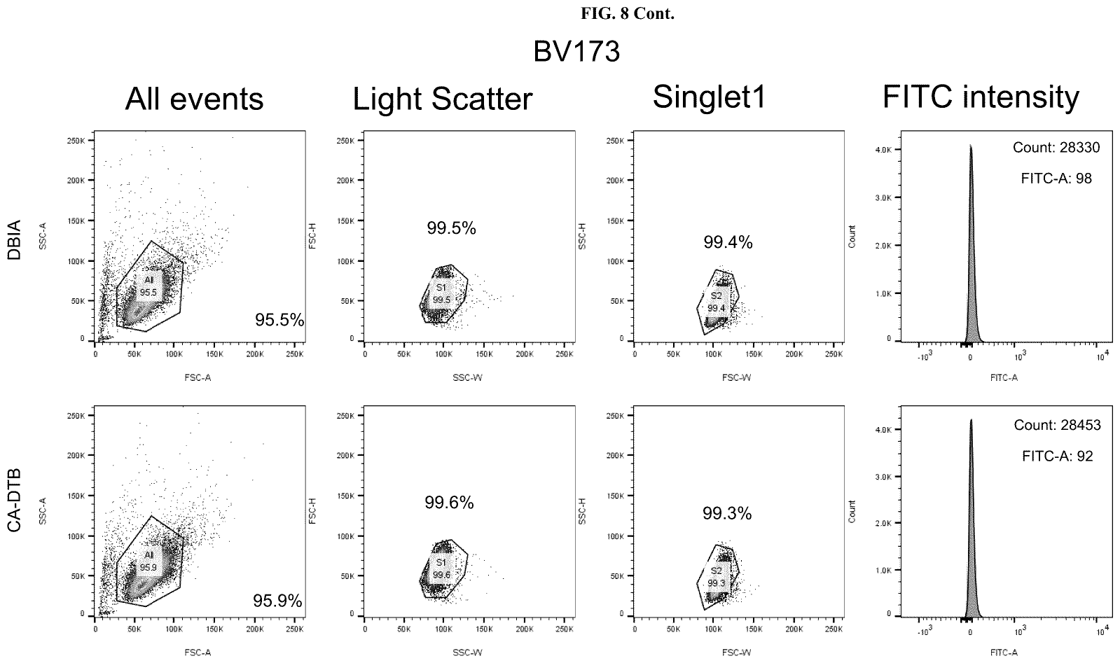

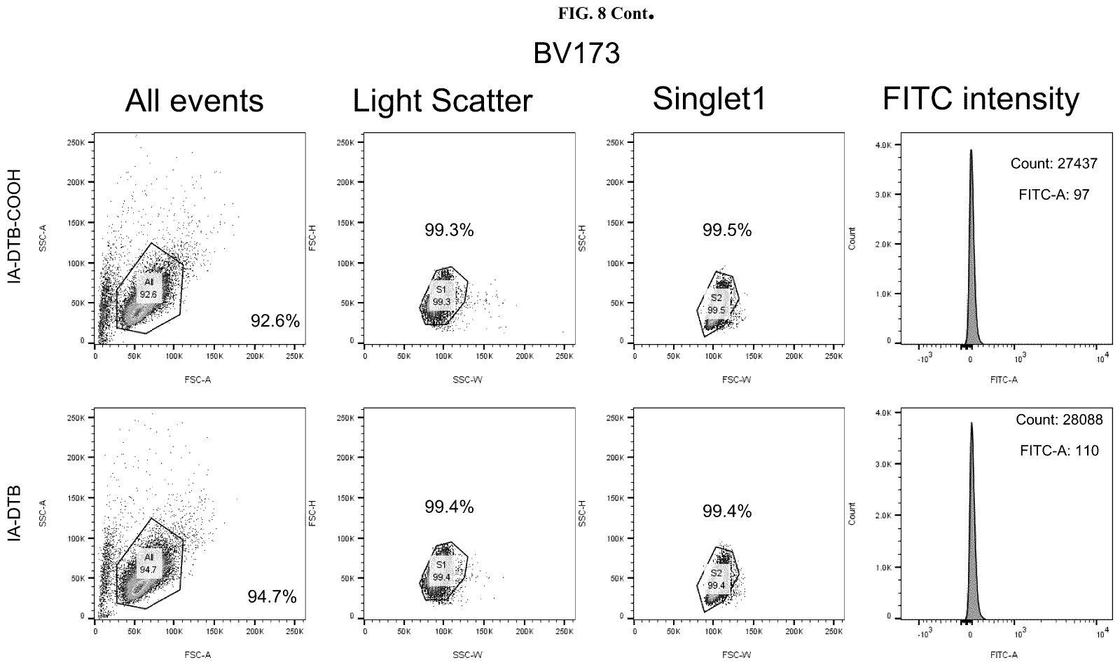

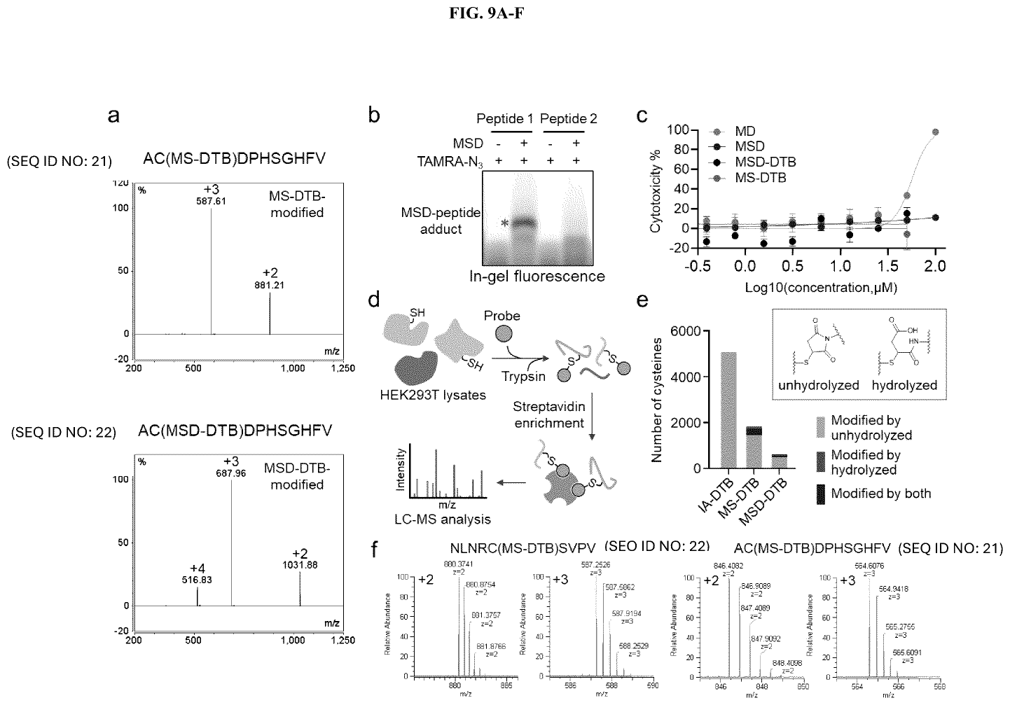

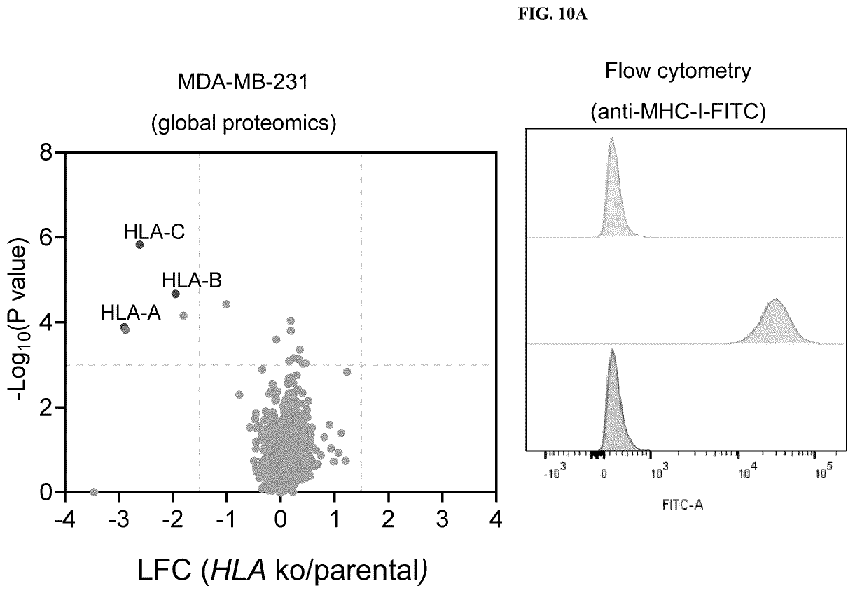

Initially, flow cytometry was used to assess cell surface labeling by these reactivity probes. BV173 (human B cell leukemia) and MT2 (human T cell leukemia) cells were treated with the probe, followed by washing out of the free probe and subsequent incubation with streptavidin-fluorescein isothiocyanate (FITC) (FIG. 1b). As streptavidin-FITC remains impermeable to cells (Juanes, M., Lostale-Seijo, I., Granja, J. R. & Montenegro, J. Supramolecular Recognition and Selective Protein Uptake by Peptide Hybrids. Chemistry 24, 10689-10698 (2018)), the fluorescence measured by flow cytometry should primarily reflect extracellular cysteine labeling by the probe. The results revealed that two sulfonated maleimide probes, maleimide-sulfonate-dibenzocyclooctyne-DTB (MSD-DTB) and maleimide-sulfonate-DTB (MS-DTB), exhibited greater cell surface labeling in both BV173 and MT2 cells compared to others (FIG. 1b and FIG. 8). To validate the specific reactivity towards cysteines by the MSD-DTB and MS-DTB probes, they were incubated with two peptides at a pH of 7.4: one containing a cysteine and the other lacking it. Liquid chromatography-mass spectrometry (LC-MS) analysis indicated that the maleimide reactive group selectively modifies cysteine residues, displaying no reactivity towards histidine and serine, and N-terminal amine (FIG. 1c and FIG. 9a). Moreover, maleimide-sulfonate-dibenzocyclooctyne (MSD, FIG. 1d) was incubated with the same two peptides, followed by an azide-alkyne cycloaddition (Kolb, H. C., Finn, M. G. & Sharpless, K. B. Click Chemistry: Diverse Chemical Function from a Few Good Reactions. Angew Chem Int Ed Engl 40, 2004-2021 (2001); Jewett, J. C. & Bertozzi, C. R. Cu-free click cycloaddition reactions in chemical biology. Chem Soc Rev 39, 1272-1279 (2010) with tetramethylrhodamine (TAMRA) azide, and observed labeling only on the cysteine-containing peptide (FIG. 9b).

To validate cell impermeability of sulfonated maleimide probes, HEK293T cells were treated with MSD and a MSD derivative with a longer linker (maleimide-sulfonate-PEG4-dibenzocyclooctyne, or MSD4), followed by fluorophore conjugation through an azide-alkyne cycloaddition and in-gel fluorescence analysis. The results indicated minimal proteome labeling compared to a cell-permeable counterpart probe, maleimide dibenzocyclooctyne (MD), that lacks the sulfonate group (FIG. 1d, e). Conversely, treating cell lysates with these reactivity probes resulted in comparable levels of proteome labeling (FIG. 1e), indicating that MSD and MSD4 probes are predominantly cell impermeable. Furthermore, it was observed that the only uncharged maleimide probe, MD, exhibited cytotoxicity compared to the other sulfonated maleimide probes (FIG. 9c). This underscores the cell impermeability of sulfonated maleimide probes incapable of accessing a wide range of intracellular proteins, potentially triggering stress and apoptosis (Jacobs, A. T. & Marnett, L. J. Systems analysis of protein modification and cellular responses induced by electrophile stress. Acc Chem Res 43, 673-683 (2010)). Finally, cysteine-directed ABPP was used to assess the global cysteine engagement of MS-DTB in cell lysates versus live cells. MS-DTB-treated samples were further labeled by DBIA for the quantification of DBIA-modified peptides. Reduced enrichment of DBIA-modified cysteine-containing peptides indicate the potential engagement of MS-DTB on these cysteines. The results revealed that among 15,310 quantified cysteines, 1,730 cysteines showed >20% engagement by MS-DTB in cell lysates, whereas only 5 cysteines showed >20% engagement by MS-DTB in live cells (FIG. 1f). This indicates that MS-DTB has restricted access to intracellular proteins. Collectively, these findings indicate that sulfonated maleimide probes are predominantly cell impermeable.

Next, the compatibility of sulfonated maleimide probes with proteomics workflow was assessed, specifically evaluating whether the probe-modified peptides can be effectively ionized and identified by orbitrap and ion trap mass analyzers. HEK293T cell lysates were incubated with MS-DTB and MSD-DTB probes, followed by trypsin digestion, enrichment with Streptavidin agarose beads, and subsequent analysis on a Tribrid mass spectrometer (FIG. 9d). The results demonstrated the effective identification of cysteines modified by both sulfonated maleimide probes (FIG. 9e), with MS-DTB probe exhibiting greater coverage than MSD-DTB probe (1,833 versus 629 probe-modified cysteine-containing peptides). This difference is likely due to the smaller size of MS-DTB (molecular weight 690.8) compared to MSD-DTB (molecular weight 993.1), which may lead to enhanced ionization and better detection in the Orbitrap mass spectrometer. Therefore, for further proteomic studies aimed at identifying probe-modified cysteine-containing peptides, MS-DTB is likely the more suitable probe. Despite carrying a negatively charged sulfonate moiety, a normal isotopic envelope profile of probe-modified peptides in positive mode on the orbitrap mass analyzer was observed (FIG. 9f). Maleimide-modified cysteines can exist in both unhydrolyzed and hydrolyzed forms (McConnell, E. W., Smythers, A. L. & Hicks, L. M. Maleimide-Based Chemical Proteomics for Quantitative Analysis of Cysteine Reactivity. J Am Soc Mass Spectrom (2020)). These data revealed that approximately 20% of probe-modified peptides contain hydrolyzed maleimide (FIG. 9e). Therefore, integrating both forms into the proteomics analysis pipeline provides a comprehensive approach.

### Example 2

### Sulfonated Maleimide Probes Label Cysteines on MHC-I-Bound Antigens

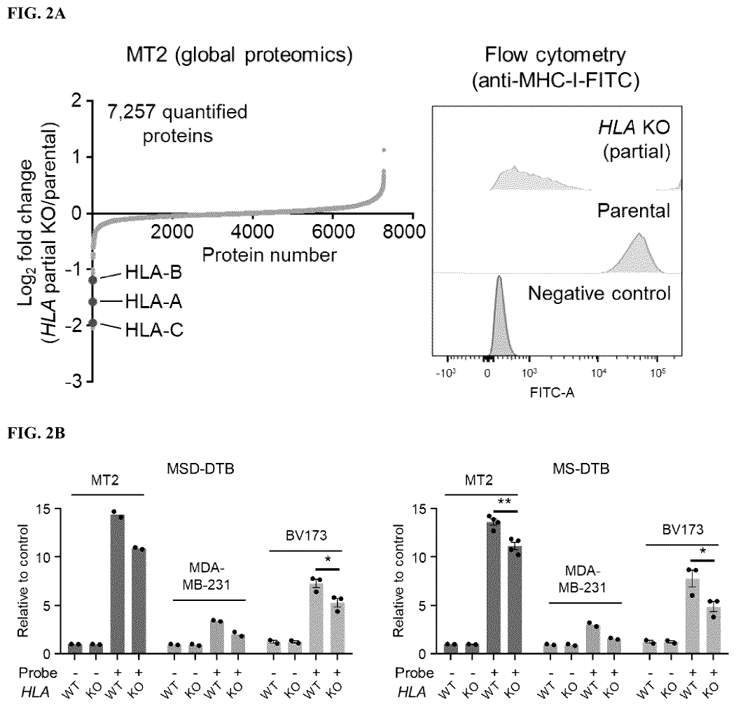

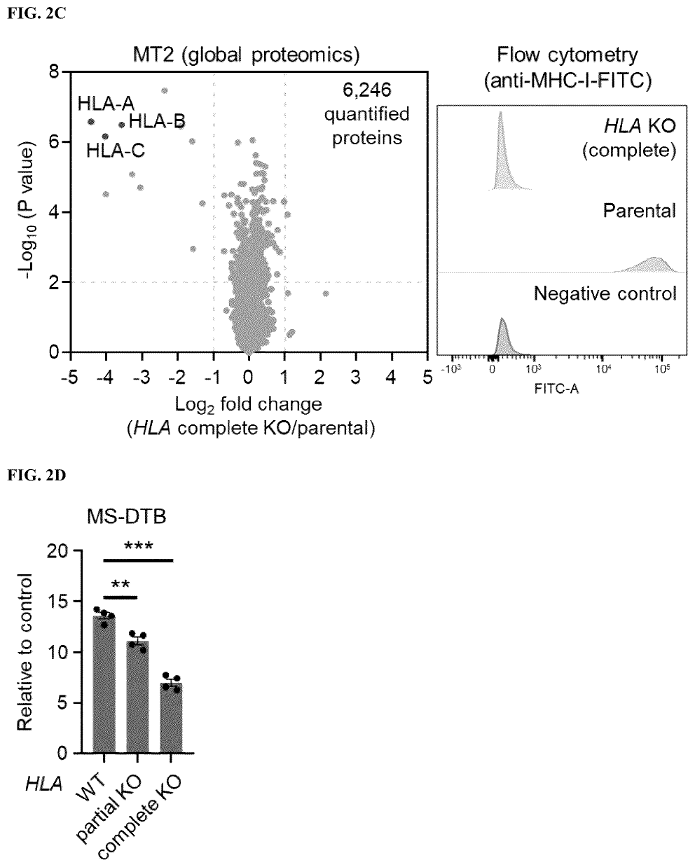

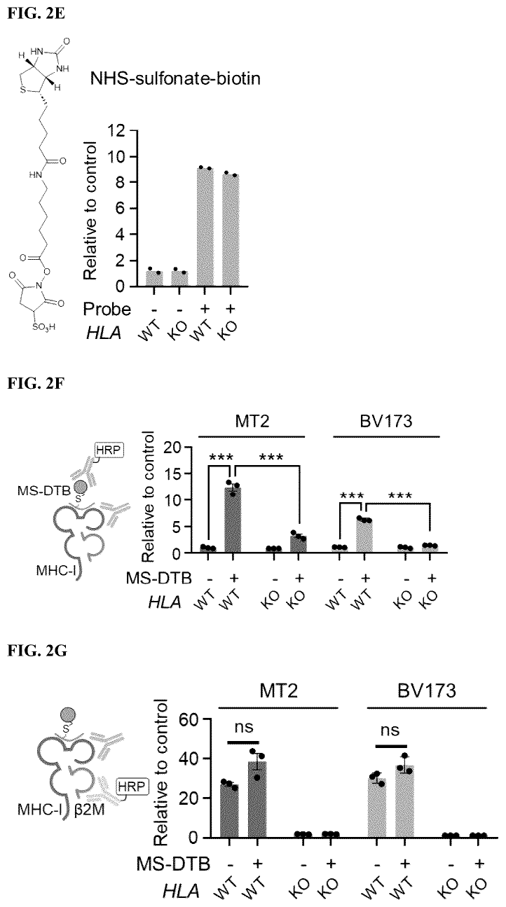

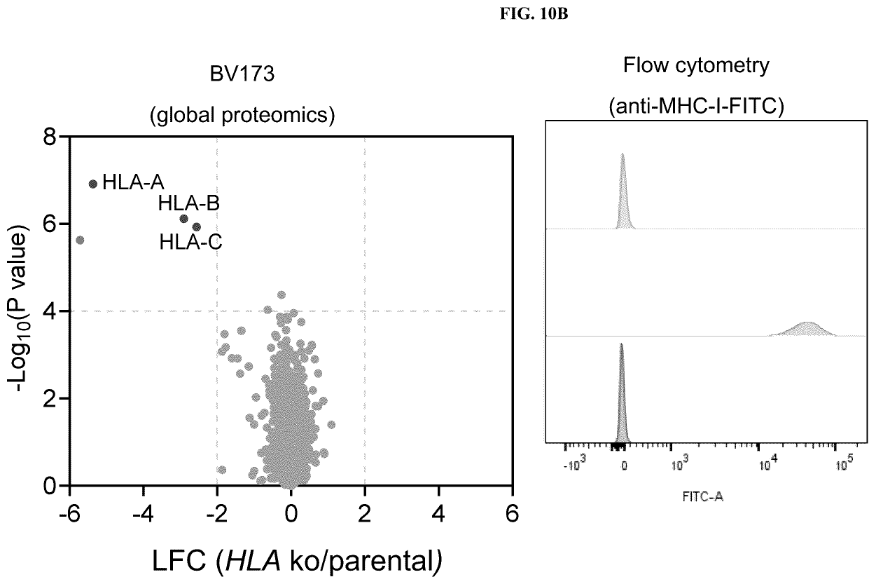

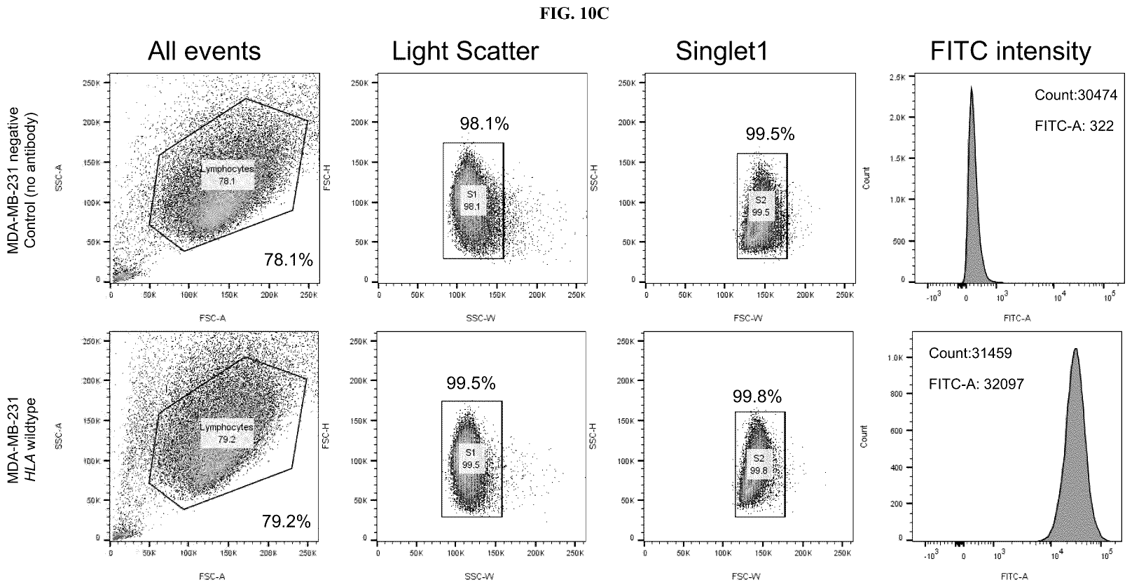

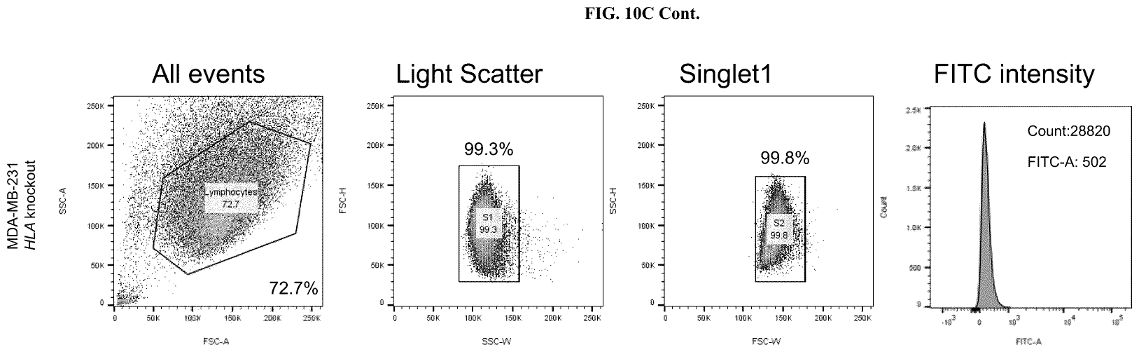

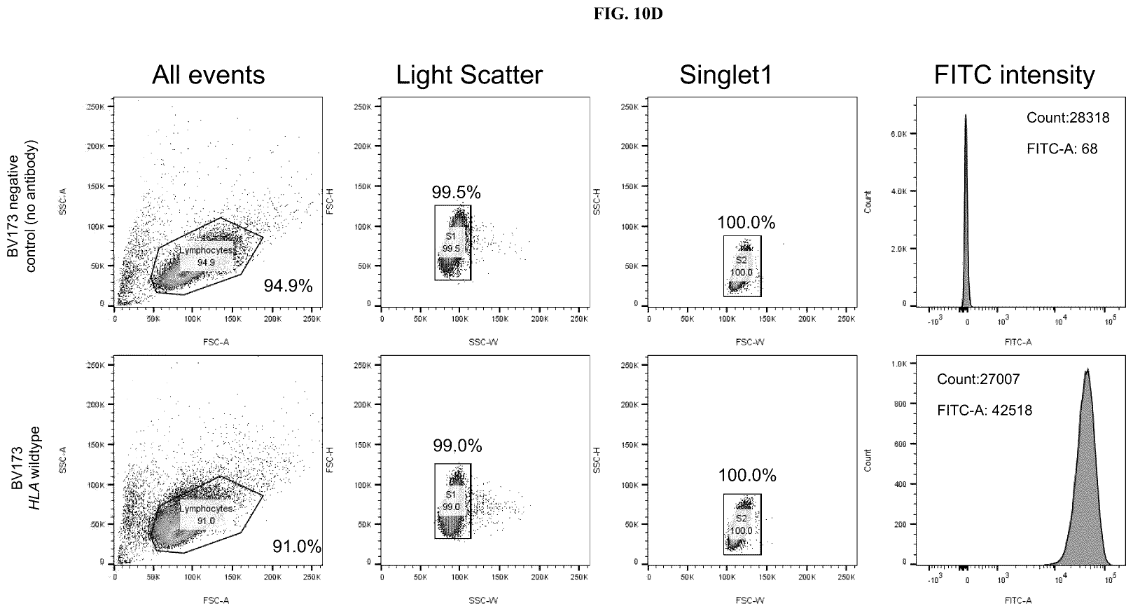

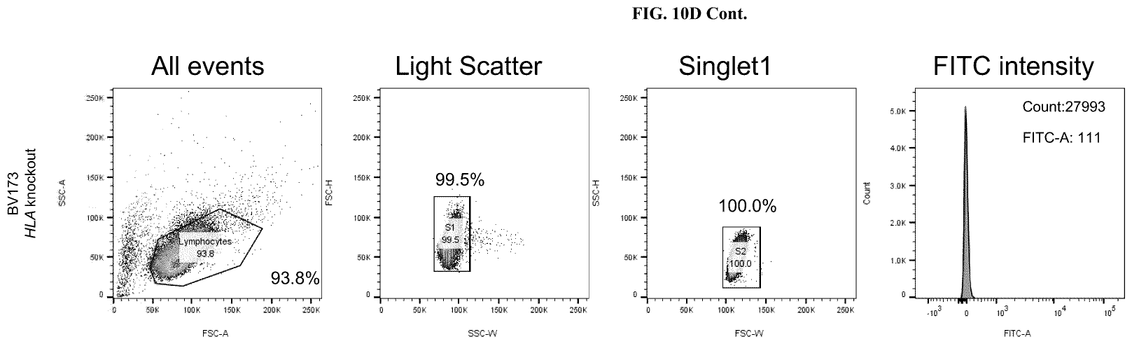

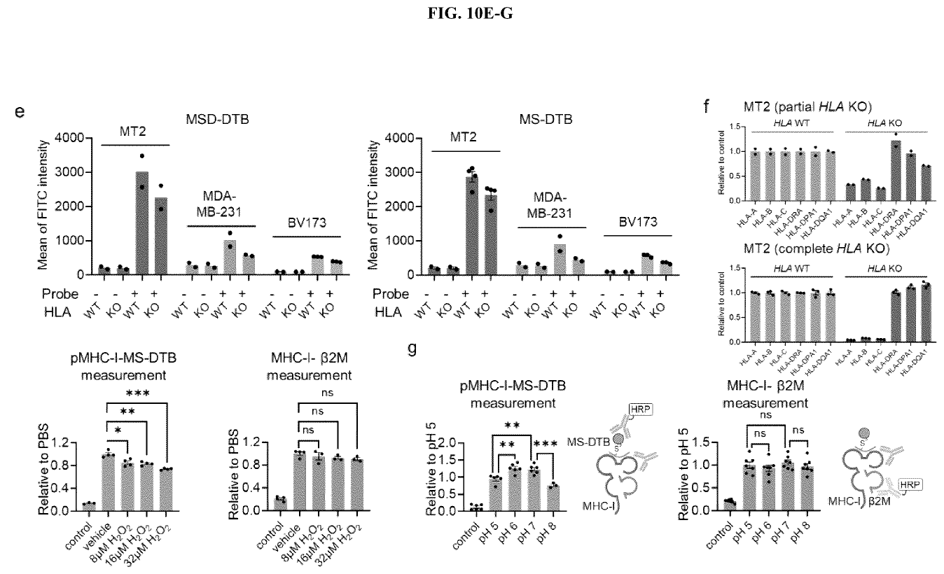

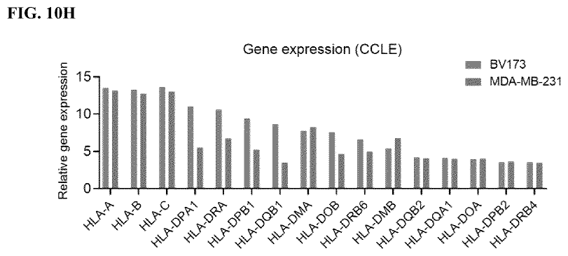

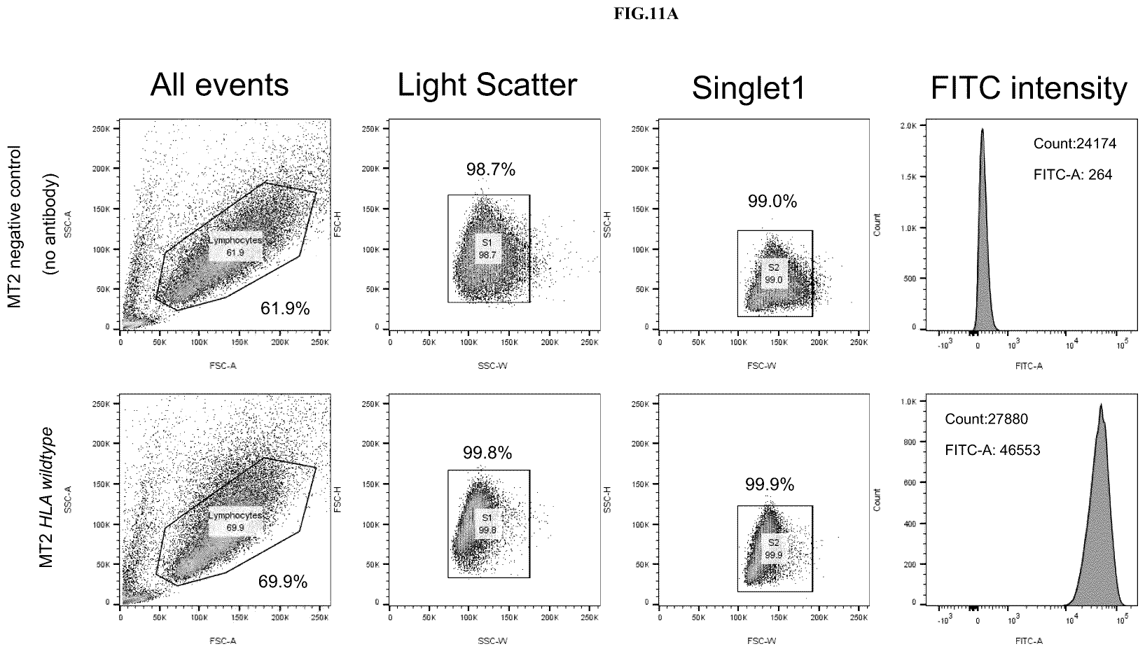

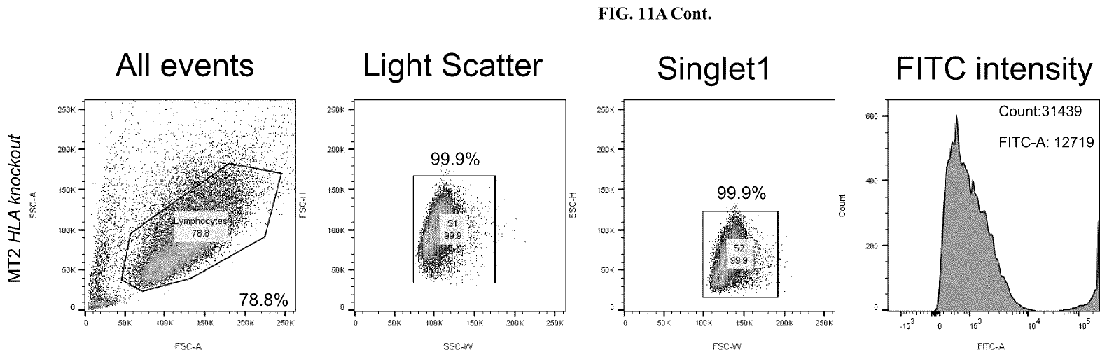

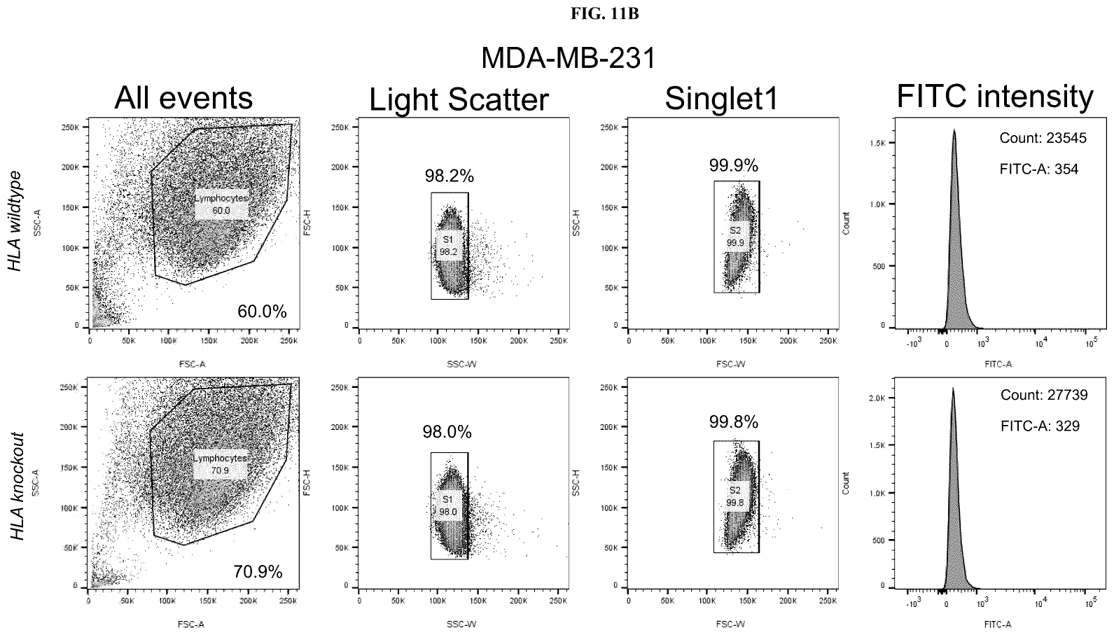

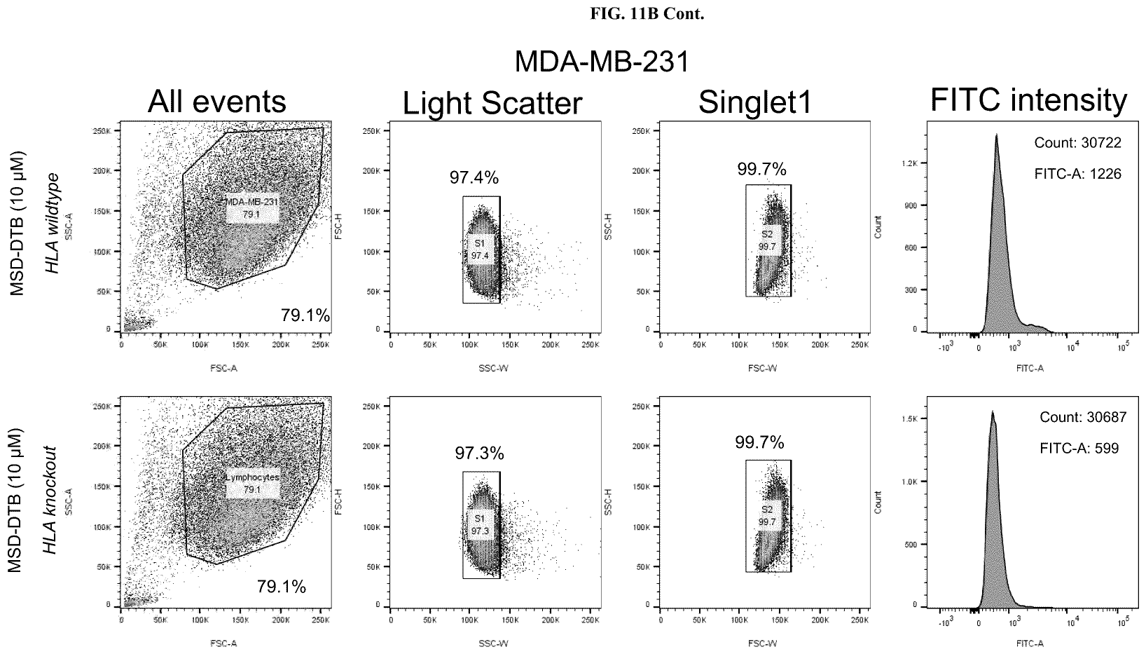

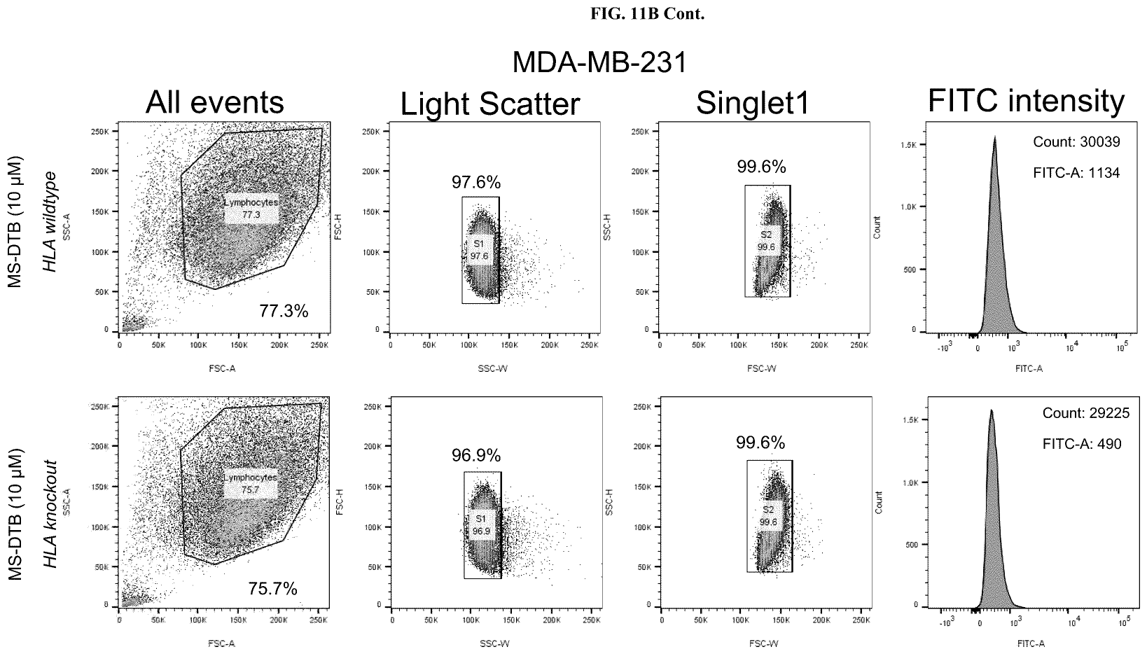

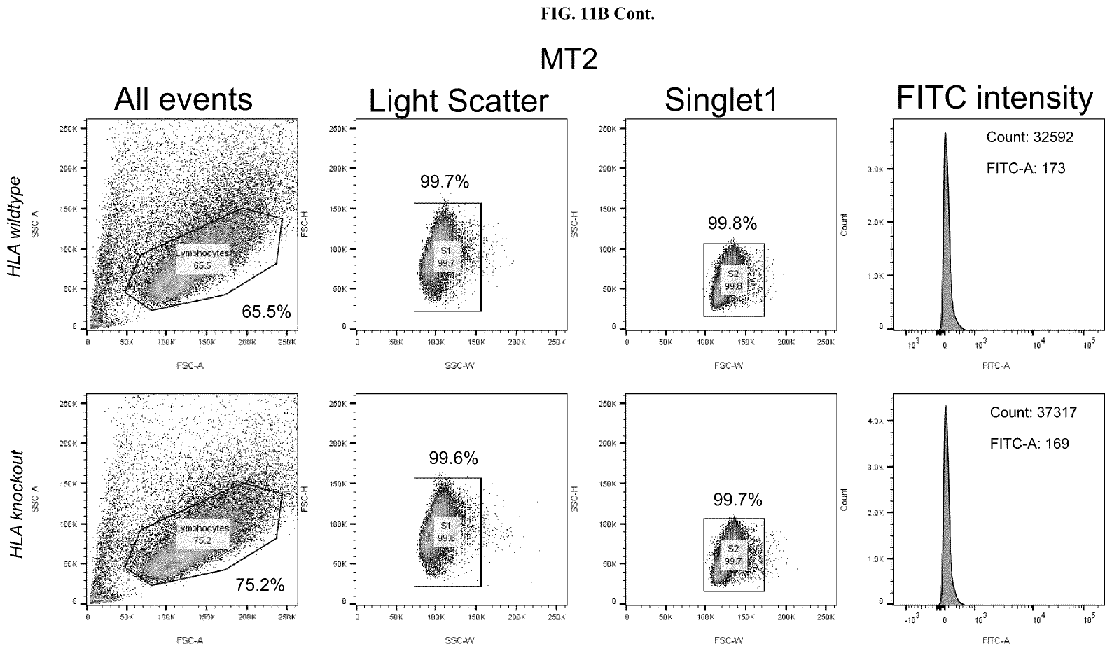

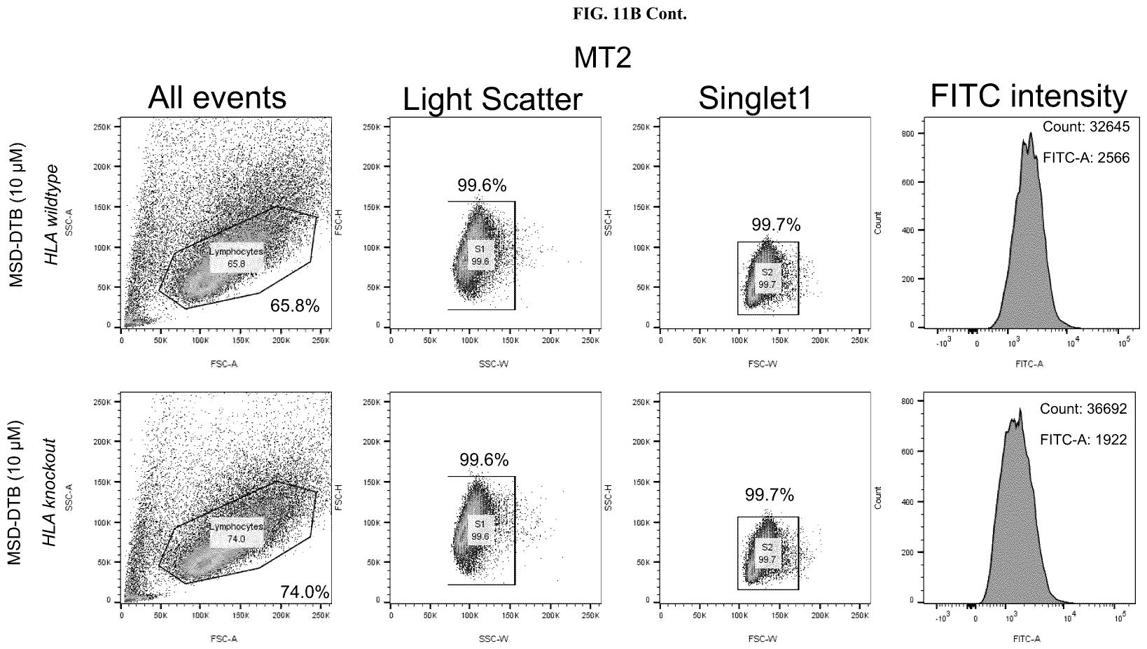

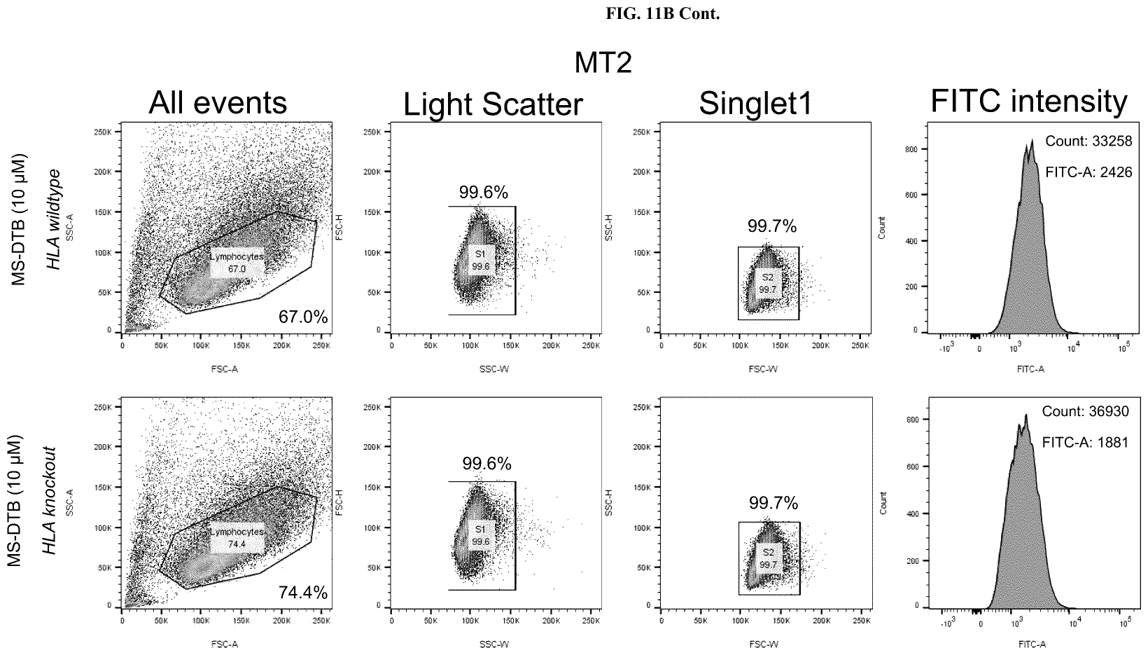

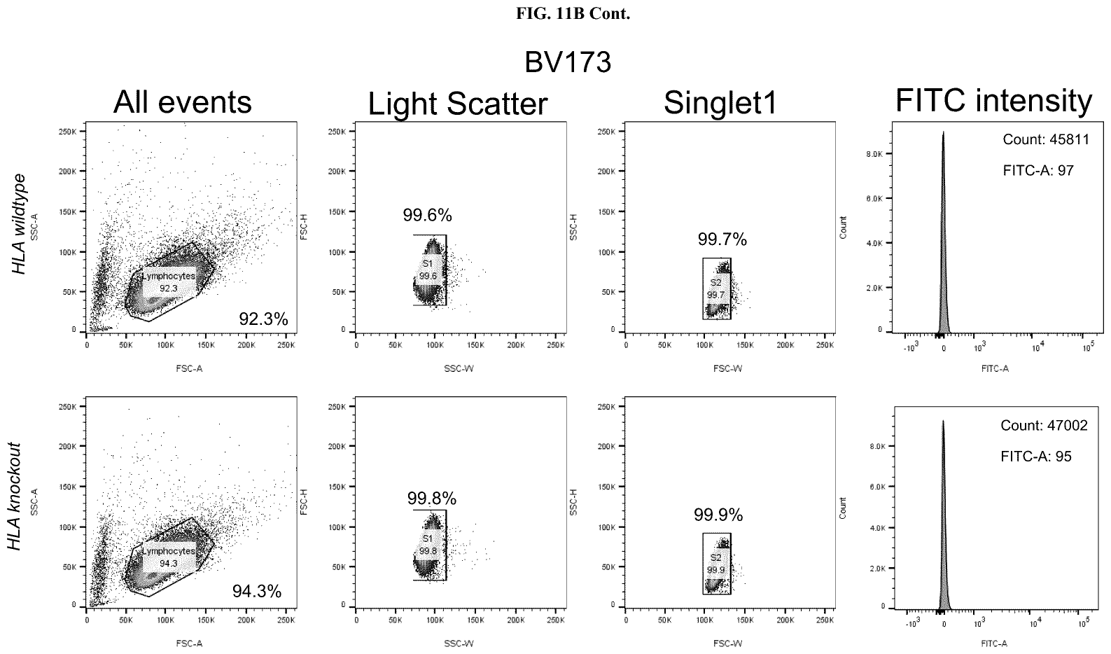

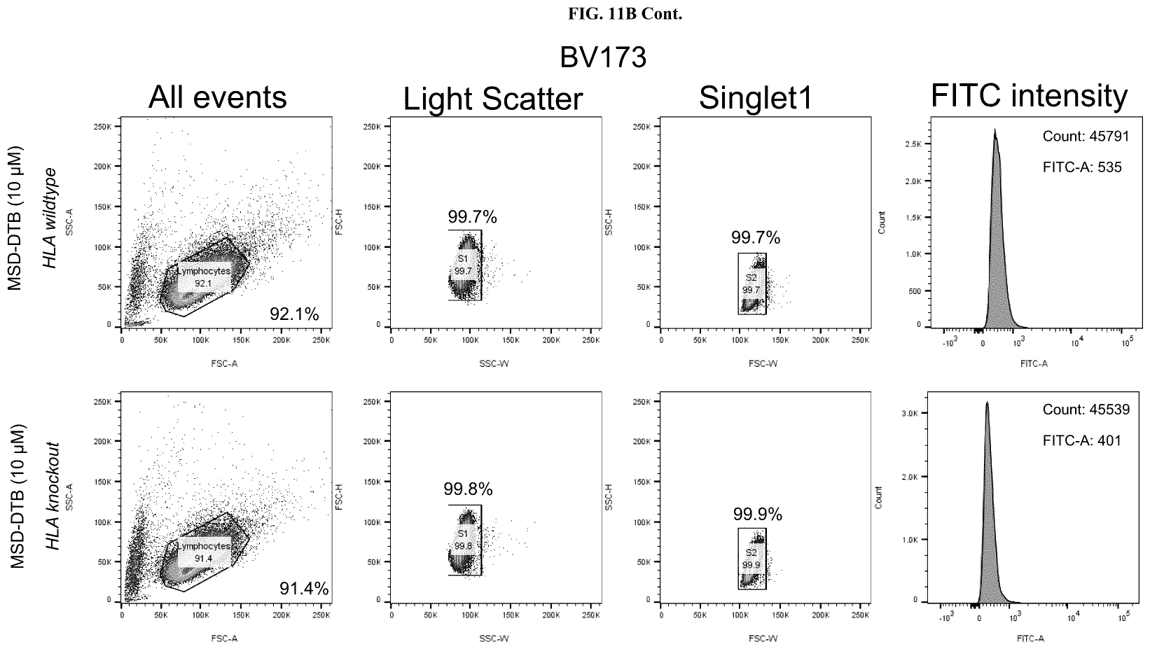

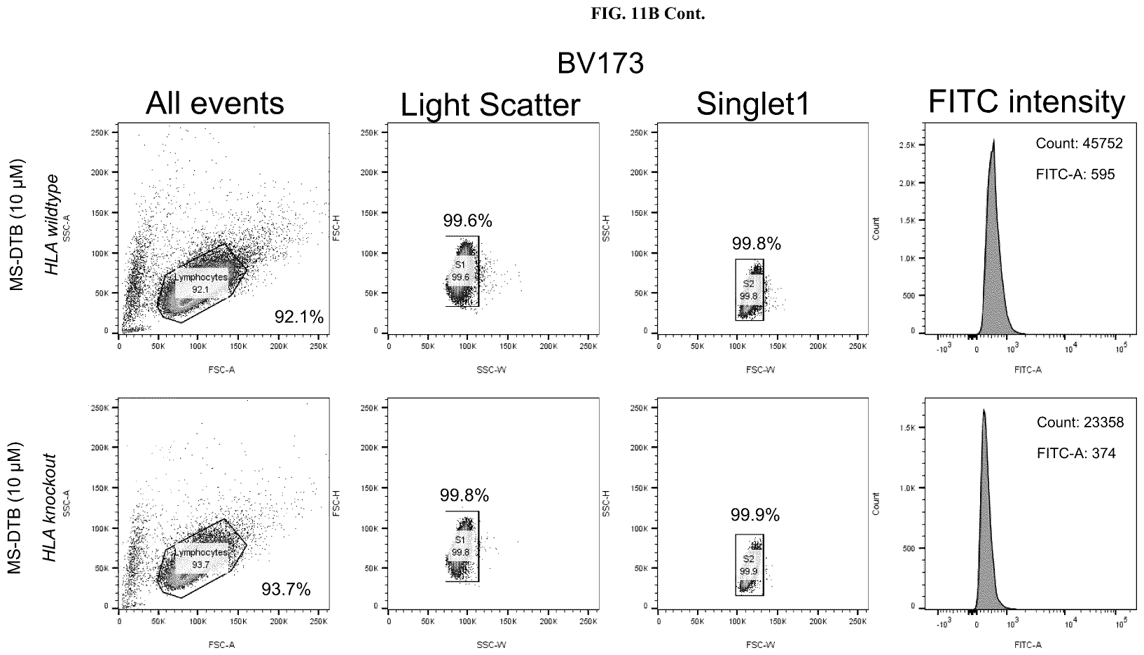

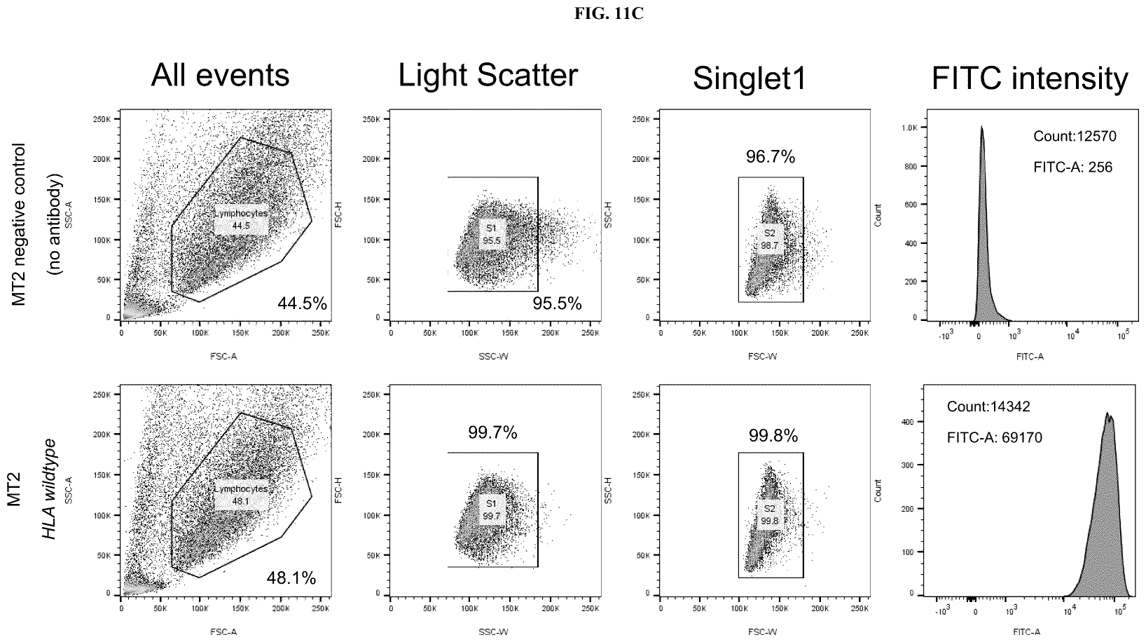

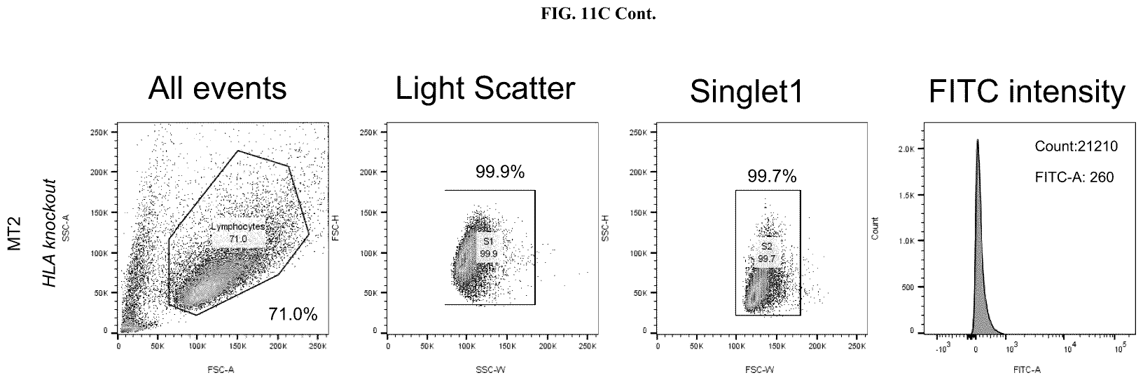

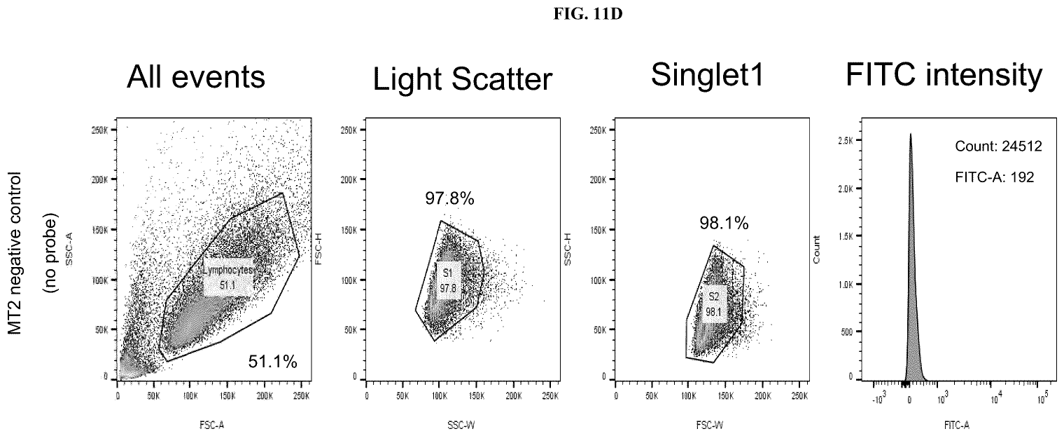

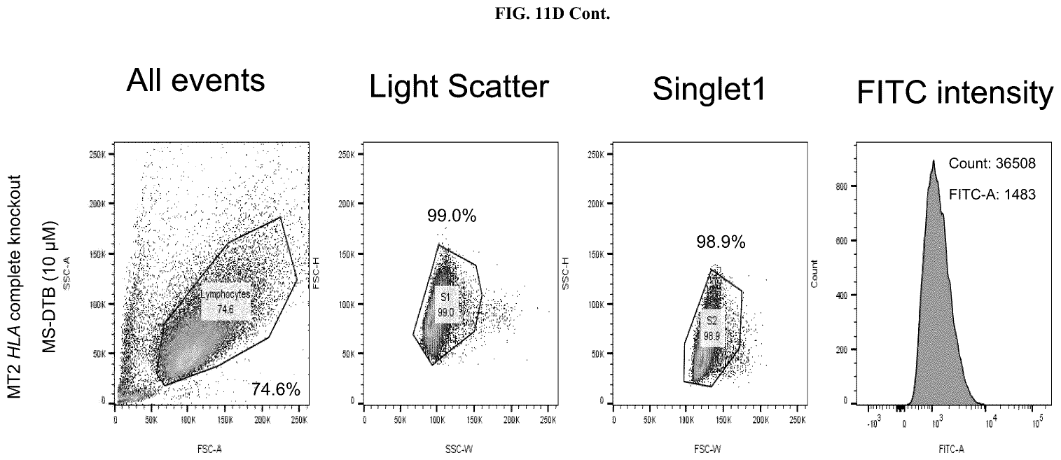

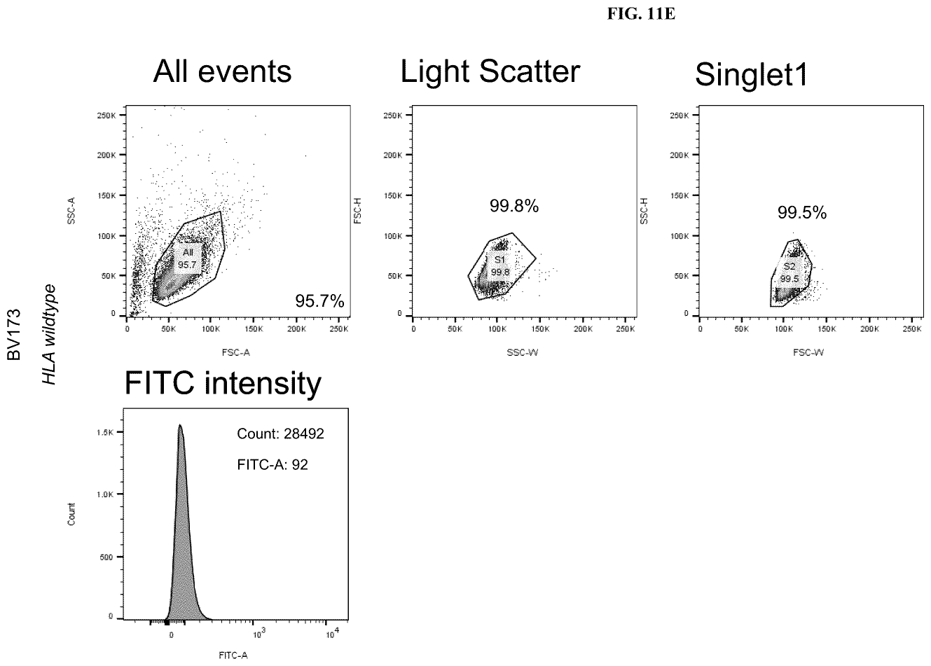

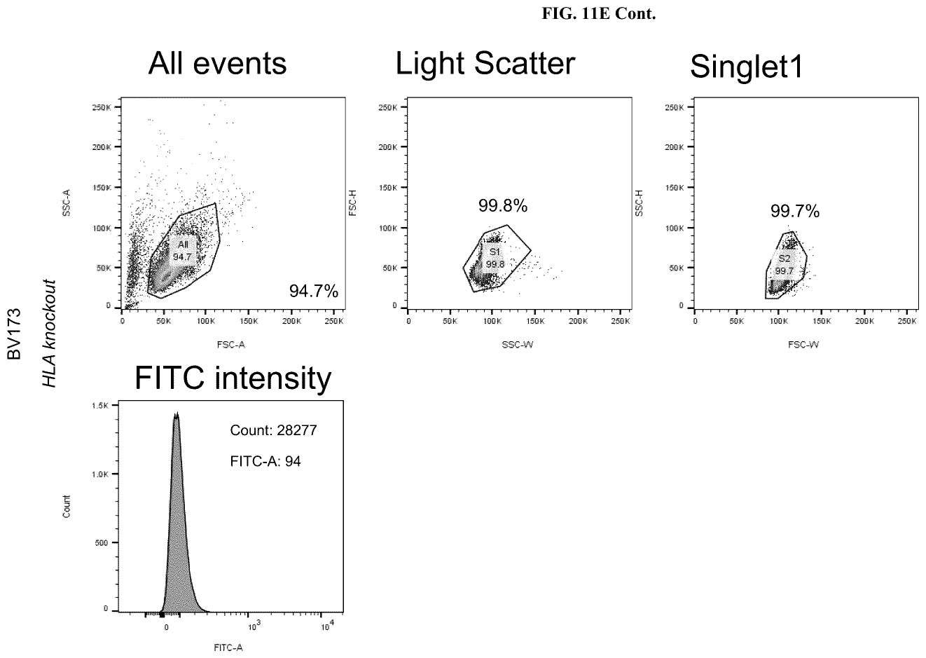

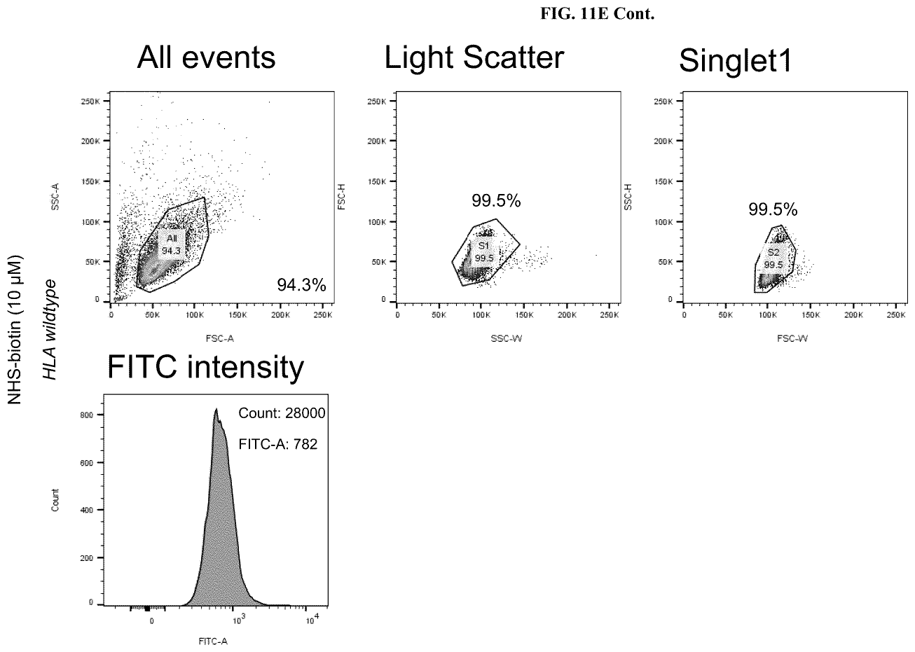

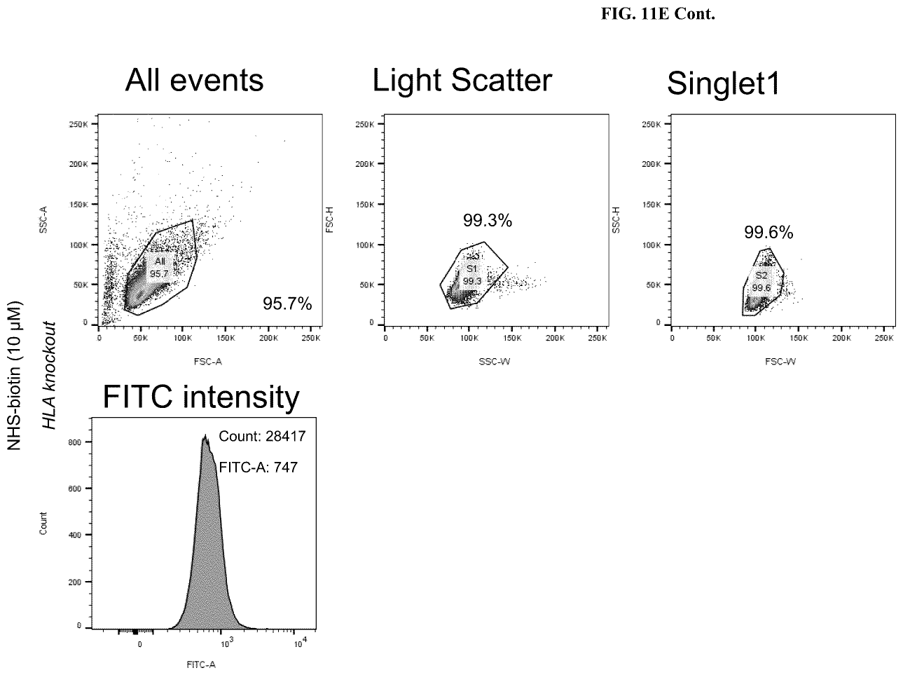

Three cell lines, BV173, MT2 and MDA-MB-231, were selected to examine the potential of sulfonated maleimide probes in capturing cysteine-containing MHC-I bound antigens. These cell lines harbor commonly occurring HLA alleles (BV173: HLA-A*02:01,30:01; MDA-MB-231: HLA-A*02:01,02:17; and MT2: HLA-A*24:02) 21, 22. Additionally, according to the TRON Cell Line Portal and Cancer Cell Line Encyclopedia, BV173 and MDA-MB-231 cells exhibit high expression levels of HLA genes and MHC-I proteins (Scholtalbers, J. et al. TCLP: an online cancer cell line catalogue integrating HLA type, predicted neo-epitopes, virus and gene expression. Genome Med 7, 118 (2015); Nusinow, D. P. et al. Quantitative Proteomics of the Cancer Cell Line Encyclopedia. Cell 180, 387-402 e316 (2020)). Proteomics and flow cytometry studies indicated that MT2 cells also exhibited high MHC-I expression levels (see below). Thus, these cell lines serve as ideal models for studying potentially abundant cysteines within the MHC-I-associated immunopeptidome. To create control cell lines, Clustered Regularly Interspaced Short Palindromic Repeats (CRISPR)-Cas9 was used to knockout all six endogenous HLA-A, HLA-B, and HLA-C genes in these cell lines (referred to as HLA knockout hereafter). The knockout of HLA genes and the disruption of MHC-I proteins were confirmed through quantitative global proteomics and flow cytometry analysis (FIG. 2a, FIG. 10a-d, FIG. 11a).

Two approaches were used to assess sulfonated maleimide probes interacting with cysteines on MHC-I-bound antigens. In the first approach, cell surface labeling by the probe in wildtype versus HLA knockout cells was compared via flow cytometry. The results revealed a decrease in cell surface labeling by both MSD-DTB and MS-DTB probes in HLA knockout cells compared to wildtype cells across all three cell lines (FIG. 2b, FIG. 10e, and FIG. 11b), indicating that the reduced cell surface labeling is likely attributable to MHC-I-bound antigens. A partial HLA knockout was noted in MT2 cells, as indicated by global proteomics and flow cytometry analyses (FIG. 2a). The MHC-I negative population was sorted in HLA partial knockout cells to generate HLA complete knockout cells (FIG. 2c, FIG. 10f, FIG. 11c). An approximately 50% decrease in cell surface labeling by MS-DTB in HLA complete knockout cells was observed, compared to a 20% decrease in HLA partial knockout cells (FIG. 2d and FIG. 11d). Furthermore, cell surface labeling was measured by a sulfonated lysine-reactive probe, N-hydroxysuccinimide (NHS)-sulfonate-biotin, and similar cell surface labeling was observed in BV173 wildtype and HLA knockout cells (FIG. 2e and FIG. 11e). Given that the majority of lysine residues in the extracellular milieu are from proteins, the cell surface labeling by the lysine-reactive probe is likely predominantly attributed to protein lysine residues and minimally affected by HLA knockout.

It was noticed that, in BV173 and MT2 HLA KO cells, MS-DTB probe still generated background cell surface labeling compared to no probe control, while in MDA-MB-231 cells, this background signal is lower (FIG. 10E). One possibility is that the probes label MHC-II-associated antigens. This hypothesis is supported by the CCLE proteomics data that MHC-II proteins in BV173 generally exhibit high expression compared to those in MDA-MB-231 cells (FIG. 10H).

In the second approach, an enzyme-linked immunosorbent assay (ELISA) was used to quantify probe-modified antigens within the pMHC-I complex. Given that sulfonated maleimide probes do not penetrate cells and both MHC-I and β2-microglobulin lack unmodified cysteines in their extracellular domains, the presence of desthiobiotin in the pMHC-I complex implies the probe-modified peptide antigens (FIG. 2f). MT2 and BV173 wildtype and HLA knockout cells were treated with the MS-DTB probe, the cells were lysed and enriched for MHC-I protein using a pan-MHC antibody conjugated on the plate. The abundance of desthiobiotin was detected using Streptavidin-horseradish peroxidase (HRP). The results revealed the presence of MS-DTB in the pMHC-I complex in wildtype cells, with significantly lower levels observed in HLA knockout cells (FIG. 2f). Moreover, the MS-DTB treatment did not affect pMHC-I folding, as indicated by the ELISA assay measuring the interaction between MHC-I and β2-microglobulin (FIG. 2g). Collectively, the findings from both approaches support that sulfonated maleimide probes modify cysteines on MHC-I-bound antigens.

With these assays, the use of the MS-DTB probe to monitor shifts in cysteine reactivity of MHC-I-bound antigens in response to changes in pH and oxidative stress was assayed. Through ELISA assays, MS-DTB probe labeling was assessed on pMHC-I at pH of 5, 6, 7, and 8. The results showed that at pH 5, probe labeling on pMHC-I was significantly reduced, while labeling remained consistent at pH 6 and 7 (FIG. 10g). At pH 8, however, probe labeling decreased dramatically, likely due to the dissociation of antigen peptides from the pMHC-I complex at this pH (Stryhn, A. et al. pH dependence of MHC class I-restricted peptide presentation. J Immunol 156, 4191-4197 (1996)). These findings indicate that pH changes can influence cysteine reactivity on MHC-I-bound antigens. Additionally, the impact of oxidative stress was examined by pretreating cells with varying concentrations of hydrogen peroxide, which is known to induce cysteine oxidation (Poole, L. B. & Nelson, K. J. Discovering mechanisms of signaling-mediated cysteine oxidation. Curr Opin Chem Biol 12, 18-24 (2008)) and, consequently, may block the reactive cysteines on MHC-I bound peptides. ELISA assays revealed decreased probe labeling on the pMHC-I complex under this condition (FIG. 10h), indicating that the MS-DTB probe effectively detects alterations in cysteine reactivity due to changes in the oxidative environment.

### Example 3

### MHC-I Immunopeptidome Exhibits Distinct Cysteine Reactivities

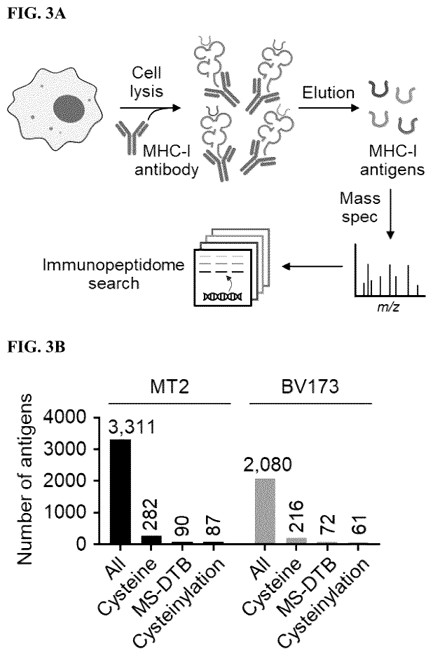

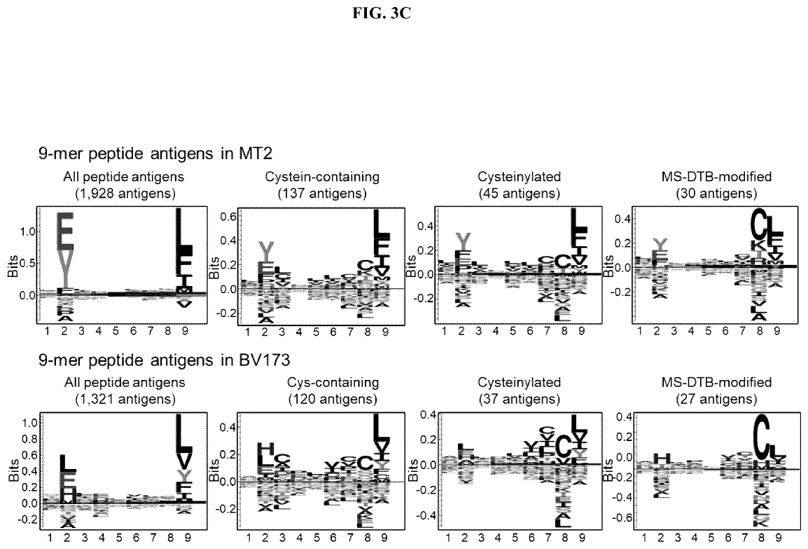

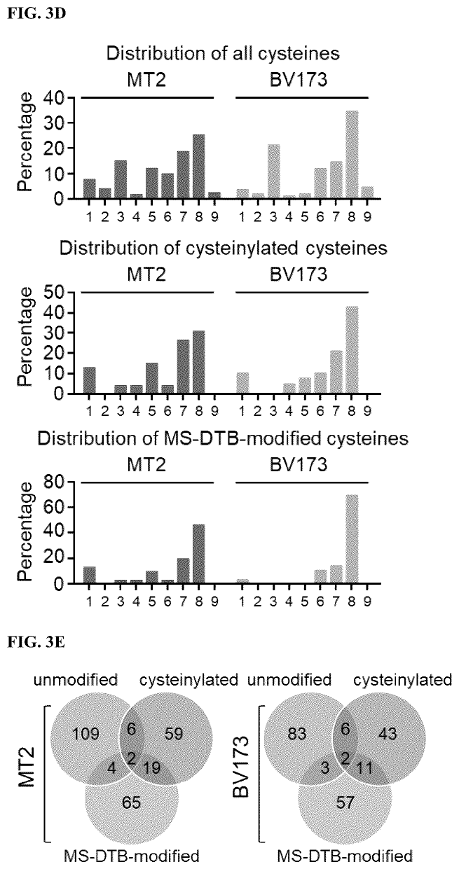

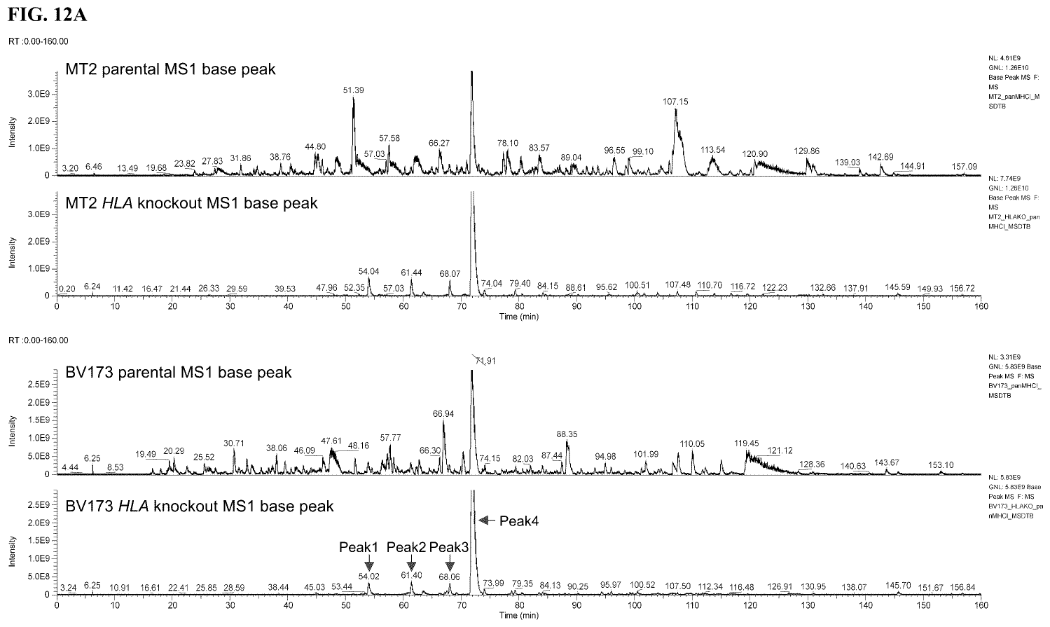

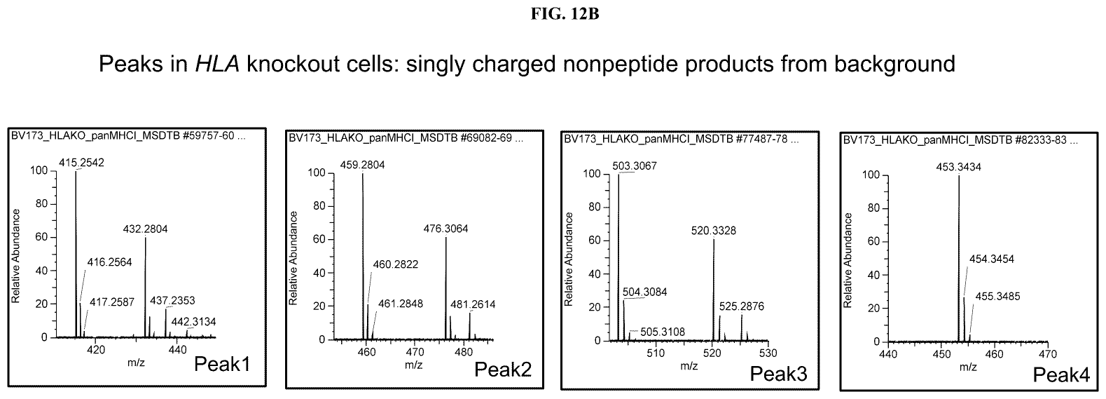

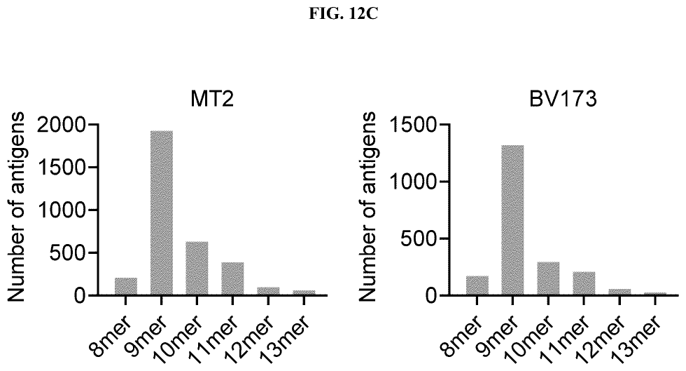

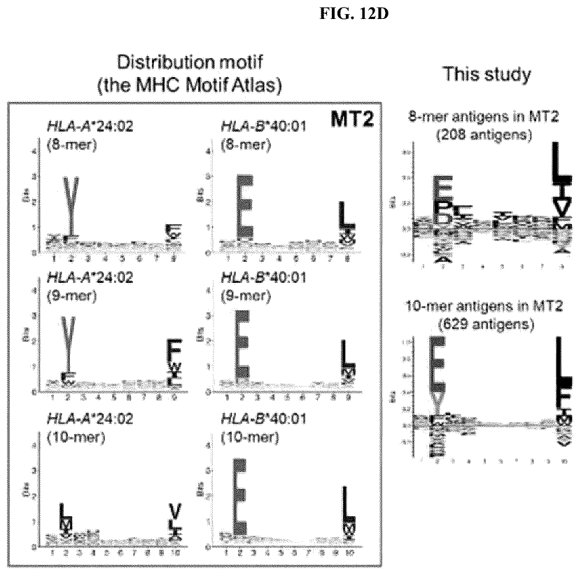

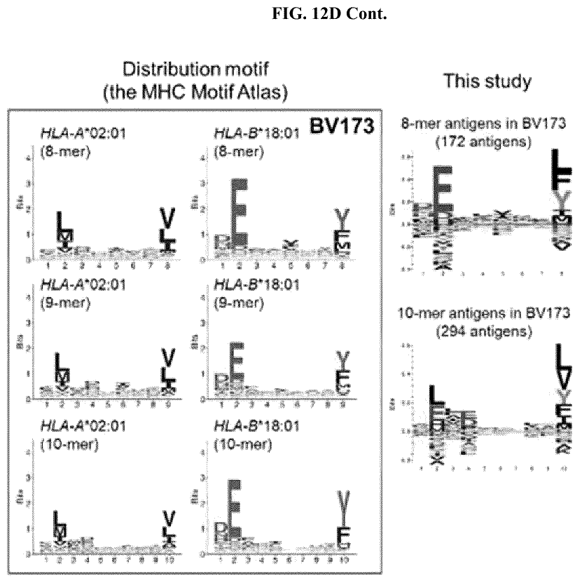

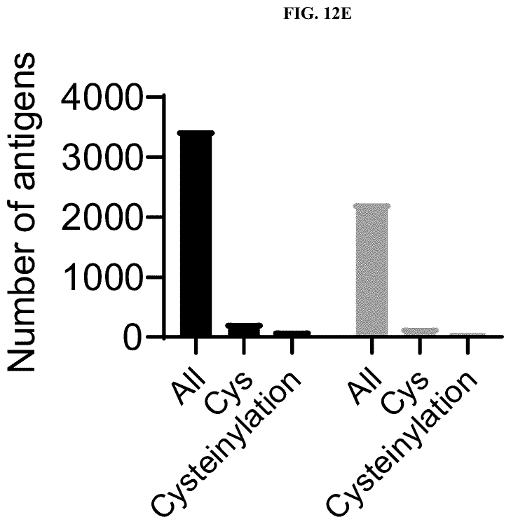

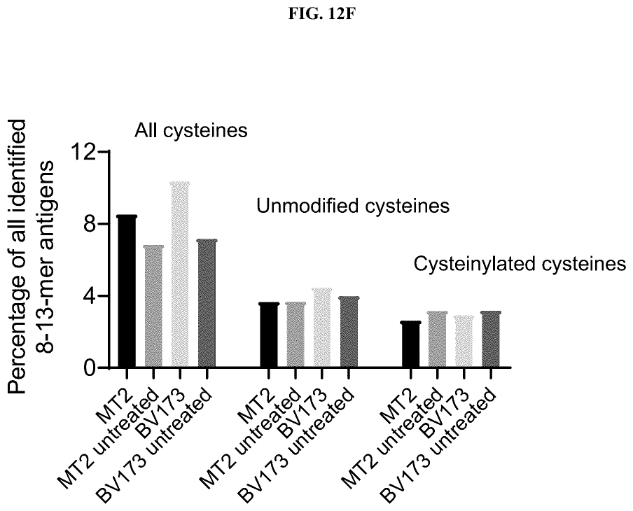

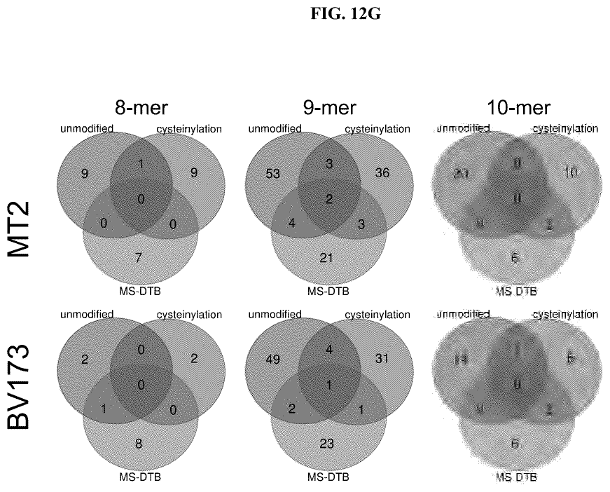

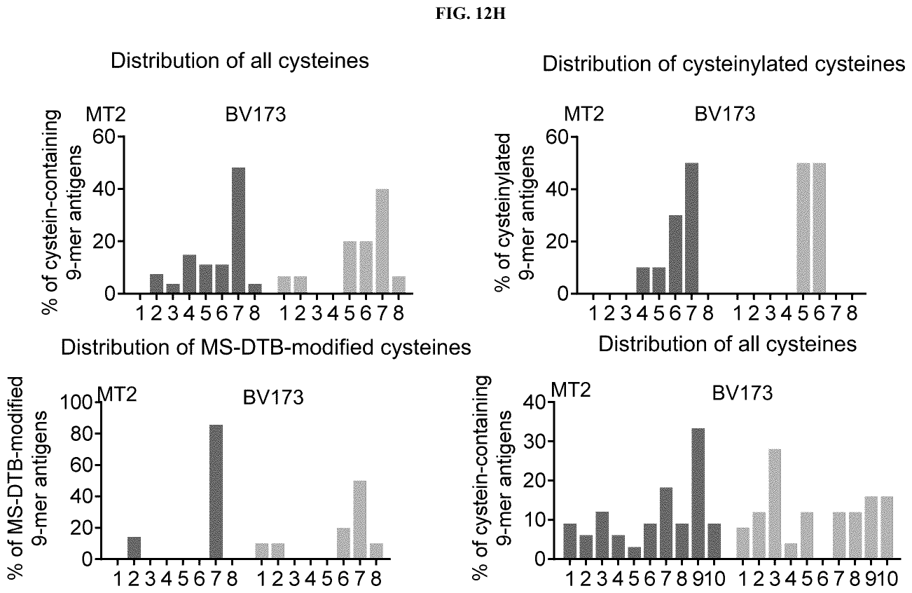

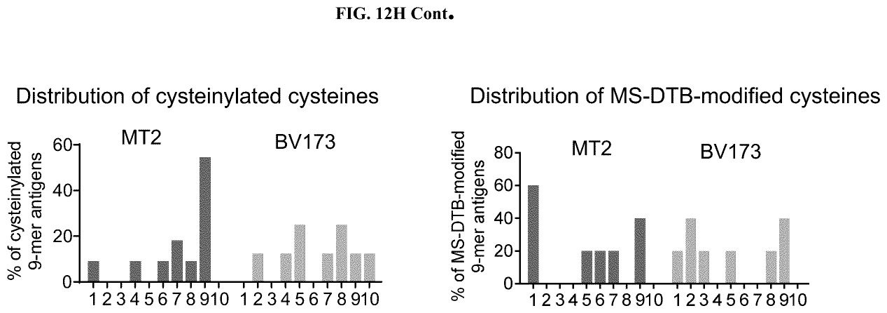

Next, immunopeptidomics was employed to examine the abundance and positioning of probe modified cysteines within the MHC-I immunopeptidome. MT2 and BV173 cells were treated with MS-DTB, then washed twice with phosphate buffered saline (PBS) before harvesting to remove free probes from the culture media. This was followed by cell lysis and immunoprecipitation using a pan-MHC-I antibody to enrich the pMHC-I complex. The immunopeptidome was eluted and analyzed using MS (FIG. 3a). During the data search, two dynamic modifications on cysteines were included: 1) MS-DTB (both unhydrolyzed and hydrolyzed forms); and 2) cysteinylation, a frequently observed cysteine modification in immunopeptidomics studies (Kacen, A. et al. Post-translational modifications reshape the antigenic landscape of the MHC I immunopeptidome in tumors. Nat Biotechnol 41, 239-251 (2023)). 3,311 and 2,080 8-13-mer peptides were identified in MT2 and BV173 cells, respectively (FIG. 3b). Parallel immunopeptidomics conducted in HLA knockout cells resulted in no 8-13-mer peptides identified in either cell line. To validate the absence of identified peptides in HLA knockout cells, the MS1 base peak chromatograms of immunopeptidomics data from HLA wildtype and knockout cells were examined. As shown in FIG. 12a, HLA wildtype cells exhibit a normal MS1 trace pattern with dispersed and individual peaks, whereas HLA knockout cells show only a few remaining peaks. The MS1 spectra of these peaks reveal singly charged nonpeptide products (FIG. 12b). No evidence of multiply charged peptide products in these peaks in HLA knockout cells was found. This low peptide background in HLA knockout cells is likely attributed to the effective HLA knockout and the use of a specific anti-MHC-I antibody (clone W6/32) in the immunopeptidomics, which generates minimal background signal.

Motif analysis of 9-mer peptides, the most preferred length for MHC-127 (FIG. 12c), revealed a close alignment with reported distribution patterns associated with the corresponding HLA alleles (FIG. 3c and FIG. 12d) (Tadros, D. M., Eggenschwiler, S., Racle, J. & Gfeller, D. The MHC Motif Atlas: a database of MHC binding specificities and ligands. Nucleic Acids Res 51, D428-D437 (2023), indicating the effective enrichment of the MHC-I immunopeptidome. Similar matched distribution patterns were also observed for 8-mer and 10-mer MHC-I bound peptides (FIG. 12d). Among all the identified antigens, 282 and 216 contain cysteine residues in MT2 and BV173 cells, respectively (FIG. 3b). Motif analysis of cysteine-containing 9-mer antigens revealed a similar pattern to that of all 9-mer antigens (FIG. 3c), indicating the confident identification of these cysteine-containing peptides binding to MHC-I. The analysis of cysteinylation on MHC-I-bound antigens revealed enrichment primarily at positions 7 and 8 (FIG. 3d), consistent with the distribution motif reported in a recent study (Kacen et al., supra). Imunopeptidomics was conducted on untreated MT2 and BV173 cells and 3,449 and 2,232 8-13-mer peptides, respectively were identified (FIG. 12e). Among these, 236 peptides in MT2 cells and 160 peptides in BV173 cells contained cysteine residues, constituting 6.8% and 7.2% of the total identified peptides in each cell line, respectively (FIG. 12f). In comparison, MS-DTB-treated cells exhibited slightly higher numbers and percentages of cysteine-containing peptides (8.5% and 10.4% in MT2 and BV173 cells, respectively).

Approximately 30% of the identified cysteines in both cell lines were found to be modified by MS-DTB. Notably, there was minimal overlap between MS-DTB-modified and unmodified cysteine-containing antigens (FIG. 3e and FIG. 12g). It was contemplated that a portion of cysteine-containing antigens may harbor unreactive cysteines that are inaccessible to even high concentration (50 μM) of maleimide probes. Conversely, the cysteines modified by MS-DTB may exist in a solvent-exposed reactive state, making them effectively trackable by maleimide probes, leading to complete labeling and the observed pattern of minimal overlap between unmodified and probe-modified antigen cysteines. Among all the MS-DTB-modified cysteines on 9-mer antigens, there is a preference for MS-DTB labeling at position 8 in both MT2 and BV173 cells (FIG. 3d). 8-mer MS-DTB-modified peptides were then analyzed and it was observed that the MS-DTB probe preferentially labeled cysteines at position 7 on 8-mer antigens (FIG. 12h). These patterns indicate that the MS-DTB probe may preferentially label the second-to-last position of MHC-I-bound antigens. For 10-mer antigens, the number of MS-DTB-labeled peptides is relatively small (five each in MT2 and BV173), which may limit the statistical representativeness of the distribution pattern. Moreover, the analysis of cysteine-containing longer antigens revealed that the majority were modified by MS-DTB (FIG. 12i). It is contemplated that these longer cysteine-containing antigens may have increased cysteine solvent accessibility, which could lead to enhanced probe labeling.

Next, overlap analyses comparing unmodified cysteines in untreated samples with MS-DTB-modified cysteines in treated samples for both MT2 and BV173 cells was conducted. A small subset of unmodified cysteines in the untreated samples were labeled by the MS-DTB probe (FIG. 12j), indicating their highly reactive state that can be detected by the probe. In contrast, the overlap analysis of unmodified cysteines in both untreated and treated samples revealed a substantially increased overlap (FIG. 12j), indicating a population of unreactive cysteines that are inaccessible to the MS-DTB probe. Notably, many MS-DTB-modified cysteines were not identified in the untreated samples in their unmodified state. It is contemplated that these cysteine-containing peptides might be unstable or undergo oxidation during sample preparation, making them difficult to identify. However, MS-DTB probe treatment may stabilize these peptides and/or prevent potential oxidation, thereby enabling their identification.

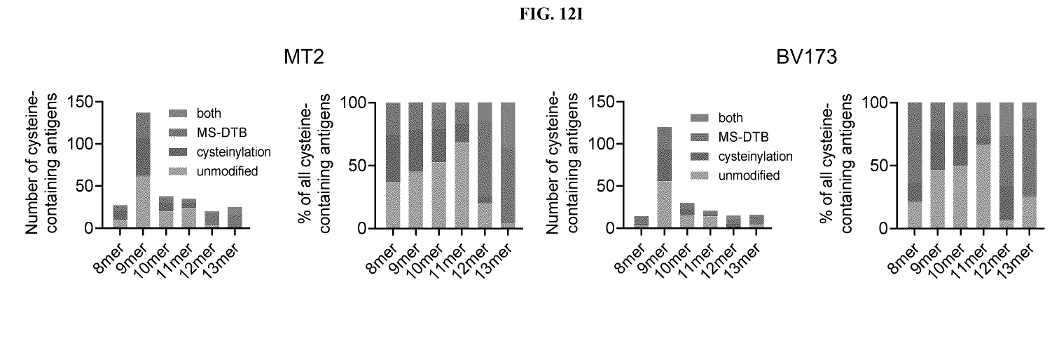

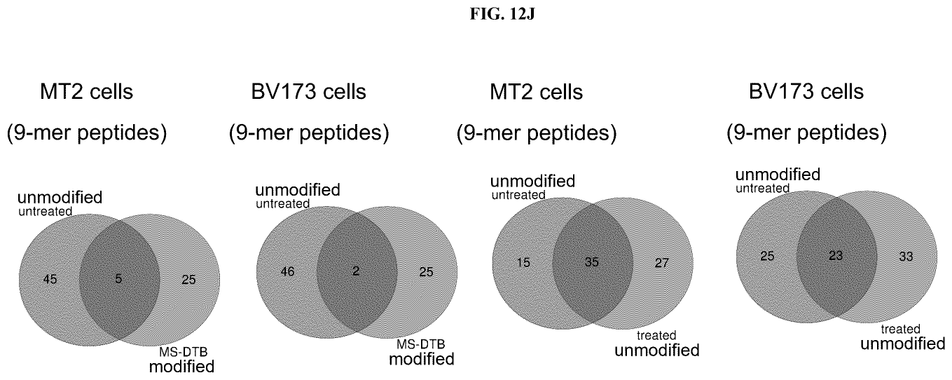

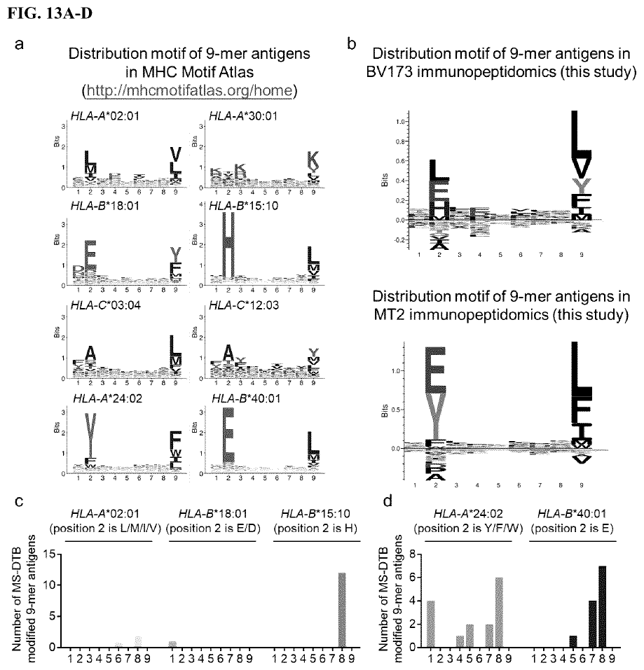

BV173 cells have well-characterized HLA class I alleles, including HLA-A*02:01, HLA-A*30:01, HLA-B*15:10, HLA-B*18:01, HLA-C*03:04, and HLA-C*12:03 (Scholtalbers, J. et al. TCLP: an online cancer cell line catalogue integrating HLA type, predicted neo-epitopes, virus and gene expression. Genome Med 7, 118 (2015)). By comparing the 9-mer antigen distribution motifs associated with these HLA alleles in the MHC Motif Atlas to those in the immunopeptidomics data, abundant expression of MHC-I proteins encoded by HLA-A*02:01, HLA-B*15:10, and HLA-B*18:01 was observed (FIG. 13a, b). Clear distribution motifs were not observed for the remaining three alleles, indicating their low abundance under the experimental conditions. In the immunopeptidomics study, although a pan-MHC-I antibody was used to enrich all pMHC-I complexes, the differential amino acids at position 2 allowed for the grouping of identified antigens according to their presumed HLA allele associations. This analysis revealed distinct distribution motifs of MS-DTB-modified 9-mer antigens associated with HLA-A*02:01, HLA-B*15:10, and HLA-B*18:01 (FIG. 13c). Notably, the majority of MS-DTB-modified peptides were associated with HLA-B*15:10. Additionally, MS-DTB exclusively labeled the second-to-last position in peptides bound to MHC-I encoded by HLA-B*15:10. These allele-driven differences may stem from variations in groove depth.

MT2 cells, on the other hand, do not have well-determined HLA class I alleles listed in public databases, but literature reports indicate they carry HLA-A*24:02 and HLA-B*40:01 (Kawamura, K. et al. Development of a Unique T Cell Receptor Gene-Transferred Tax-Redirected T Cell Immunotherapy for Adult T Cell Leukemia. Biol Blood Marrow Transplant 26, 1377-1385 (2020)). The distribution motif of 9-mer antigens associated with these two alleles in the MHC Motif Atlas aligns with the observations from the immunopeptidomics study (FIG. 13a, b). The data did not reveal additional peptides associated with HLA alleles beyond HLA-A*24:02 and HLA-B*40:01. Therefore, it is likely that MT2 predominantly expresses MHC-I proteins encoded by HLA-A*24:02 and HLA-B*40:01. Analysis of the immunopeptidomics data revealed that the MS-DTB probe labels peptides associated with HLA-A*24:02 and HLA-B*40:01 with similar efficacy (FIG. 13d). The distribution patterns of probe-modified cysteines are largely consistent between both alleles, with preferential labeling at the second-to-last position in each case. However, the MS-DTB probe labels cysteine at position 1 in peptides bound to HLA-A*24:02 but not HLA-B*40:01. One explanation may be differences in groove conformation that result in a less reactive state for cysteines at position 1 in peptides associated with HLA-B*40:01. It is contemplated that the negatively charged glutamate at position 2 in peptides associated with HLA-B*40:01 may repel the negatively charged MS-DTB probe, preventing interaction with adjacent residues.

Recent studies reveal that post-translational modifications (PTMs) can occur on MHC-I-bound antigens (Kacen et al., supra). To investigate this, abundant PTMs reported on pMHC-I, including lysine acetylation, lysine dimethylation, arginine dimethylation, asparagine deamidation, serine phosphorylation, and threonine phosphorylation, were exampled in immunopeptidomics studies. Only a few of these PTMs co-occurred with antigens modified by MS-DTB.

### Example 4

### Assessment of Reactive Cysteine Alternations in the Immunopeptidome

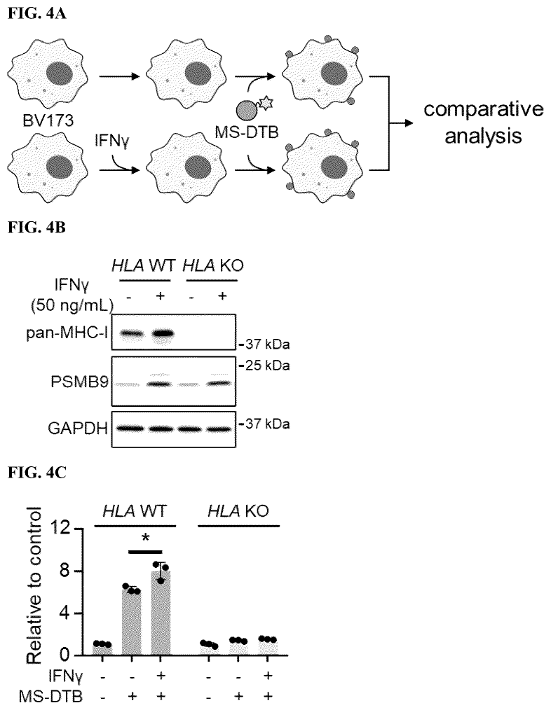

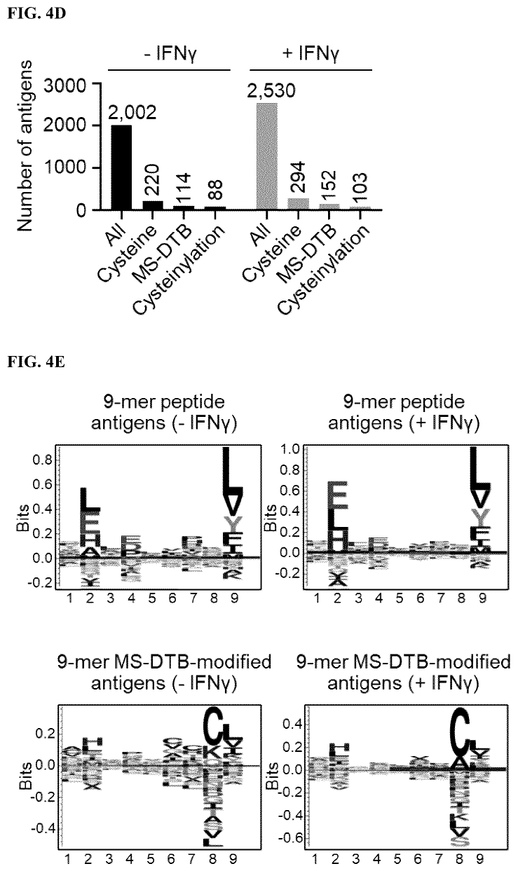

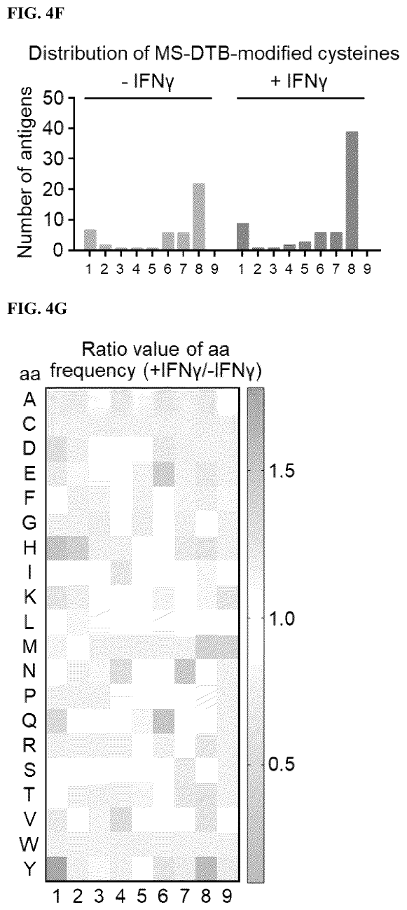

MHC-I antigen presentation undergoes regulation through various mechanisms occurring during both transcriptional and post-translational stages (Neefjes, J., Jongsma, M. L., Paul, P. & Bakke, O. Towards a systems understanding of MHC class I and MHC class II antigen presentation. Nat Rev Immunol 11, 823-836 (2011)). For example, immunoproteasome expression can influence the antigens presented by MHC-131. To explore potential alterations in reactive cysteines on MHC-I-bound antigens, BV173 cells were treated with interferon-gamma (IFNγ), a cytokine known to stimulate immunoproteasome expression (FIG. 4a). It was confirmed that IFNγ-induced immunoproteasome expression by observing increased levels of PSMB9, a subunit of the immunoproteasome (FIG. 4b) 31. A moderate increase in MHC-I expression was noted. Subsequently, using ELISA assays, significantly enhanced MS-DTB engagement within the pMHC-I complex was observed in HLA wildtype, but not knockout cells (FIG. 4c). This increased engagement may result from increased cysteine reactivity or abundance.

Further investigation via immunopeptidomics revealed 2,530 8-13-mer MHC-I-bound peptides in IFNγ-stimulated cells versus 2,002 in non-stimulated cells (FIG. 4d), indicating an overall enhancement of MHC-I presented antigens with IFNγ stimulation. Consequently, the number of MS-DTB-modified 8-13-mer peptides also increased upon IFNγ simulation (FIG. 4d). Motif analysis of 9-mer MHC-I-bound peptides comparing IFNγ-stimulated versus non-stimulated cells showed a consistent pattern (FIG. 4e), indicating that IFNγ stimulation does not alter the overall pattern of MHC-I presented antigens. Interestingly, IFNγ stimulation primarily increased MS-DTB labeling at position 8 of 9-mer MHC-I-bound peptides, with other modified positions remaining unaffected (FIG. 4f, g). Collectively, these findings demonstrate the use of sulfonated maleimide probes in mapping changes in reactive cysteines on MHC-I-associated peptide antigens during altered physiological processes.

### Example 5

### Mapping Reactive Cysteines Using the Single-Chain Trimer

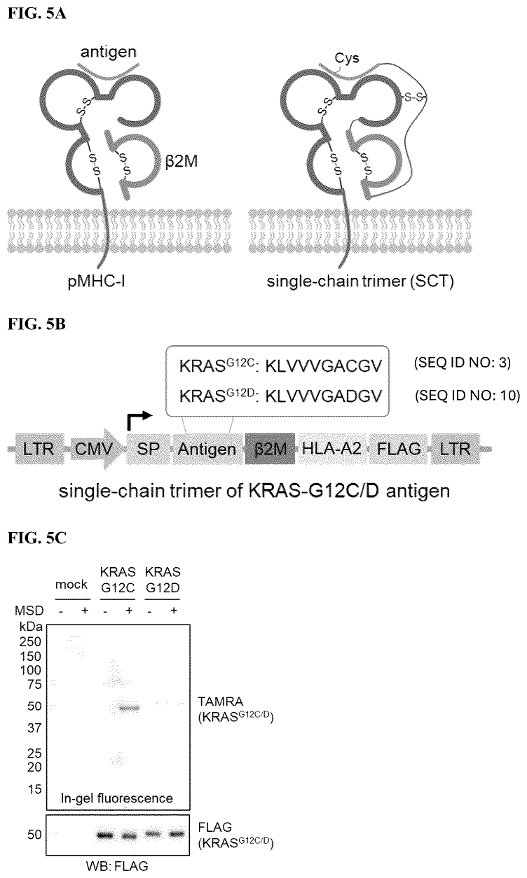

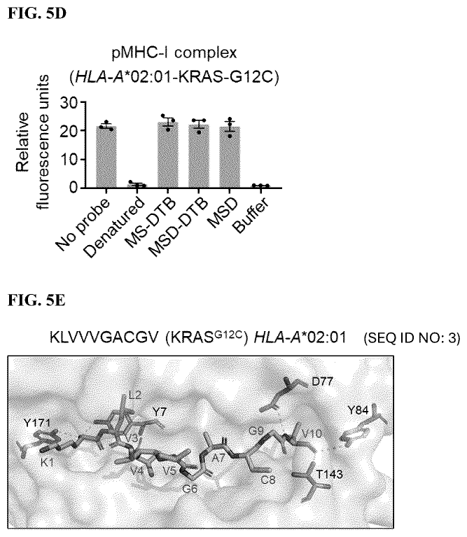

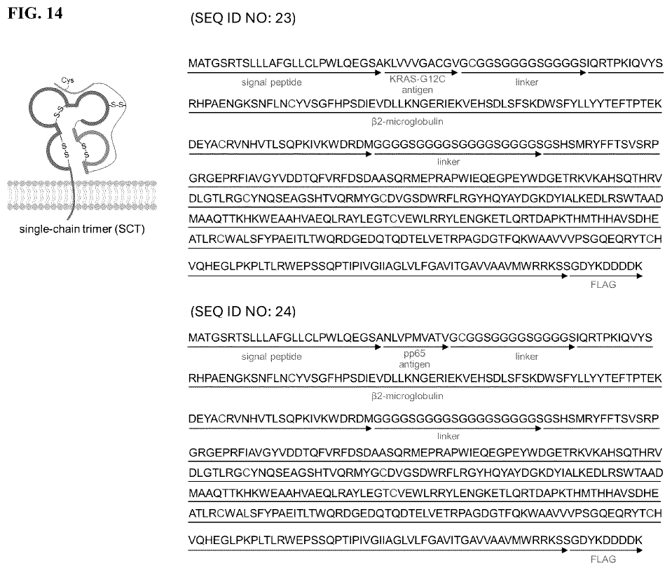

Single-chain trimers (SCTs) are engineered constructs comprising covalently linked single chains of MHC-I, β2-microglobulin, and displayed antigenic peptides (FIG. 5a) (Hansen, T., Yu, Y. Y. L. & Fremont, D. H. Preparation of stable single-chain trimers engineered with peptide, beta2 microglobulin, and MHC heavy chain. Curr Protoc Immunol Chapter 17, 17 15 11-17 15 17 (2009)). SCTs retain structural integrity similar to native pMHC-I complexes and can activate T cells (Greten, T. F. et al. Peptide-beta2-microglobulin-MHC fusion molecules bind antigen-specific T cells and can be used for multivalent MHC-Ig complexes. J Immunol Methods 271, 125-135 (2002); Yu, Y. Y., Netuschil, N., Lybarger, L., Connolly, J. M. & Hansen, T. H. Cutting edge: single-chain trimers of MHC class I molecules form stable structures that potently stimulate antigen-specific T cells and B cells. J Immunol 168, 3145-3149 (2002)), making them valuable models for investigating MHC-I antigen presentation and function. In this study, employing SCTs enables the precise generation of constructs encoding pMHC-I that present specific cysteine-containing antigens. To this end, two SCTs of HLA-A*02:01 presenting previously reported KRAS neoantigens: one containing the G12C mutation (KLVVVGACGV; SEQ ID NO: 3) (Hattori, T. et al. Creating MHC-Restricted Neoantigens with Covalent Inhibitors That Can Be Targeted by Immune Therapy. Cancer Discov 13, 132-145 (2023); Zhang, Z. et al. A covalent inhibitor of K-Ras (G12C) induces MHC class I presentation of haptenated peptide neoepitopes targetable by immunotherapy. Cancer Cell 40, 1060-1069 e1067 (2022)) and the other containing the G12D mutation (KLVVVGADGV; SEQ ID NO: 10) (Linette, G. P., Bear, A. S. & Carreno, B. M. Facts and hopes in immunotherapy strategies targeting antigens derived from KRAS mutations. Clin Cancer Res (2024)) (FIG. 5b). Both SCTs were incorporated with a C-terminal intracellular FLAG tag. HEK293T cells were transfected with KRAS-G12C- or KRAS-G12D-SCT and treated with the MSD probe. After cell lysis, SCTs were enriched via FLAG immunoprecipitation, followed by an azide-alkyne cycloaddition (Kolb, H. C., Finn, M. G. & Sharpless, K. B. Click Chemistry: Diverse Chemical Function from a Few Good Reactions. Angew Chem Int Ed Engl 40, 2004-2021 (2001); Jewett, J. C. & Bertozzi, C. R. Cu-free click cycloaddition reactions in chemical biology. Chem Soc Rev 39, 1272-1279 (2010)) with TAMRA azide, enabling visualization of MSD-modified SCTs via in-gel fluorescence. The results revealed that KRAS-G12C-SCT was labeled by the MSD probe, whereas KRAS-G12D-SCT showed no detectable labeling (FIG. 5c). Since there are no extracellular unmodified cysteines on MHC-I, β2-microglobulin, and linker components in the SCT constructs (FIG. 14), the labeling signal is likely attributed to the cysteine in the KRAS-G12C neoantigen. Furthermore, an ELISA assay was conducted by incubating sulfonated maleimide probes with recombinant pMHC-I (HLA-A*02:01) bound to the KRAS-G12C neoantigen (KLVVVGACGV; SEQ ID NO: 3). The data indicate that probe incubation did not disrupt the proper folding of the pMHC-I complex (FIG. 5d). Modeling studies using HLA-A*02:01 revealed that Y7 and Y171 on MHC-I formed hydrogen bonds with the N-terminal NH of KRAS-G12C neoantigen. On the other end, D77, Y84 and T143 in the F-pocket of MHC-I formed hydrogen bonds with the C-terminal valine (FIG. 5e). The overall confirmation of the KRAS-G12C neoantigen exposures cysteine at position 8 to the solvent, resulting in its reactive state.

Subsequently, a non-cysteine 9-mer antigen (NLVPMVATV; SEQ ID NO: 11), known as pp65 viral antigen, derived from cytomegalovirus and presented by MHC-I encoded by HLA-A*02:0137 was selected for further analysis. The SCT model was used to explore the reactivity of cysteines individually introduced at all positions of the pp65 antigen (pp65-C1-C9, FIG. 5f). The results revealed relatively low expressions of SCTs when cysteines were introduced at the anchoring positions 2 and 9, indicating that the presence of cysteines at these sites may disrupt the proper folding of the complex. Notably, MSD labeling patterns exhibited variations among these cysteine-containing antigens (FIG. 5f). For instance, pp65-C2 showed strong probe labeling despite low expression levels. In contrast, pp65-C3 showed minimal labeling despite having the highest expression levels. These findings indicate that cysteines on MHC-I-bound antigens may exhibit distinct reactivities, and the sulfonated maleimide probes are capable of mapping these variations using the SCT model. The highest labeling observed at pp65-C6 differs from the pattern observed in the immunopeptidomics studies, which indicate the highest probe labeling at positions adjacent to the anchoring residue (FIG. 3d and FIG. 13h). This discrepancy may result from the statistical likelihood of a probe-labeled cysteine being adjacent to the anchor residue. However, individual antigens may not always adhere to this pattern and can present exceptions.

### Example 6

### Reactivity-Based Profiling of Cysteines in the Immunopeptidome

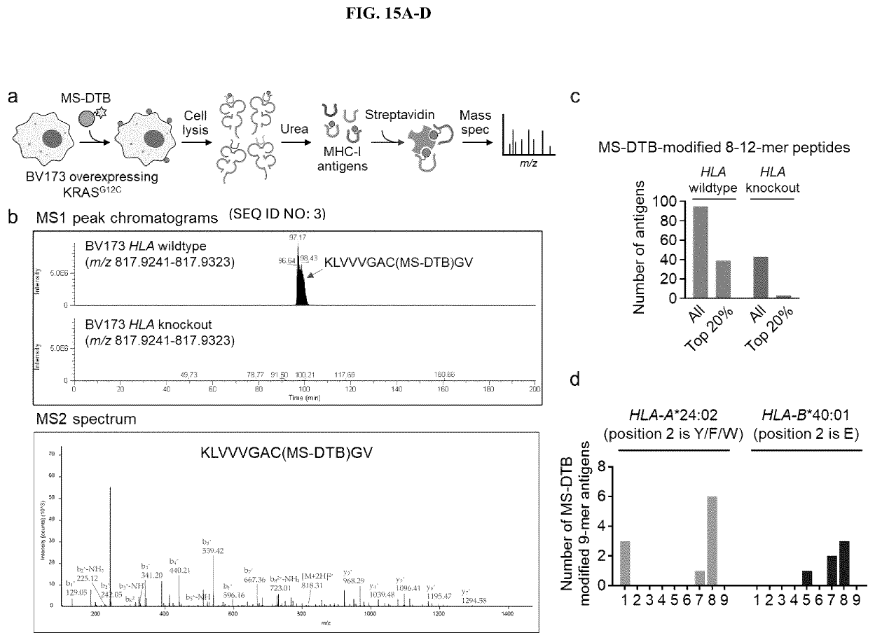

Building on the principle of cysteine-directed ABPP (Vinogradova, E. V. et al. An Activity-Guided Map of Electrophile-Cysteine Interactions in Primary Human T Cells. Cell 182, 1009-1026 e1029 (2020); Backus, K. M. et al. Proteome-wide covalent ligand discovery in native biological systems. Nature 534, 570-574 (2016)) it was explored whether sulfonated maleimide probes could facilitate global mapping of reactive cysteines on MHC-I-bound antigens. A chemical proteomics strategy wherein cells are treated with the MS-DTB probe, followed by cell lysis, streptavidin enrichment, and MS analysis was used to identify reactive cysteines on MHC-I-bound antigens. To validate this platform, KRAS-G12C was overexpressed in BV173 parental and HLA knockout cells and treated with MS-DTB (FIG. 15a). Subsequent proteomic analysis revealed the identification of MS-DTB-modified KRAS-G12C neoantigen only in BV173 parental cells, but not in HLA knockout cells (FIG. 15b), indicating the utility of this approach to profile reactive antigen cysteines in an untagged manner.

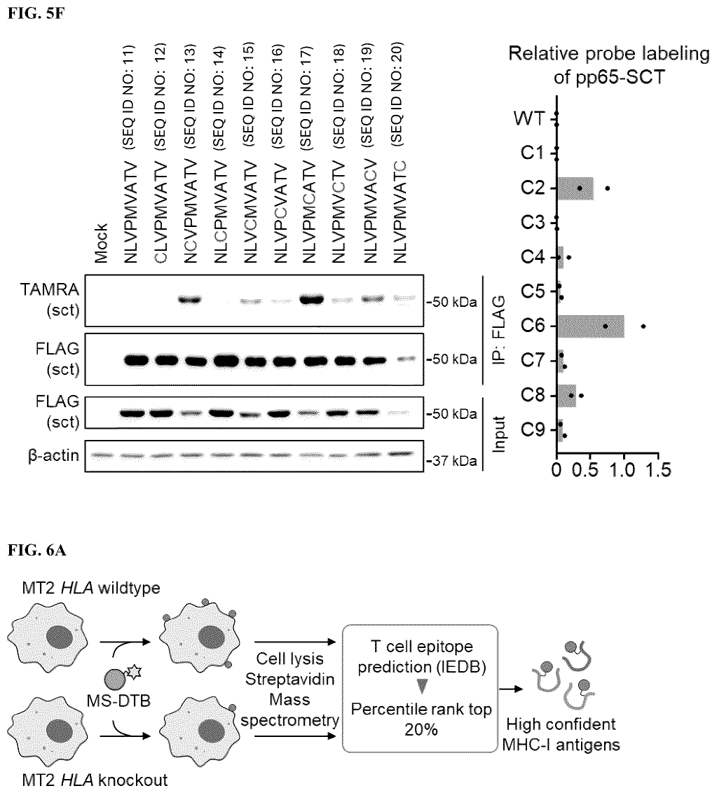

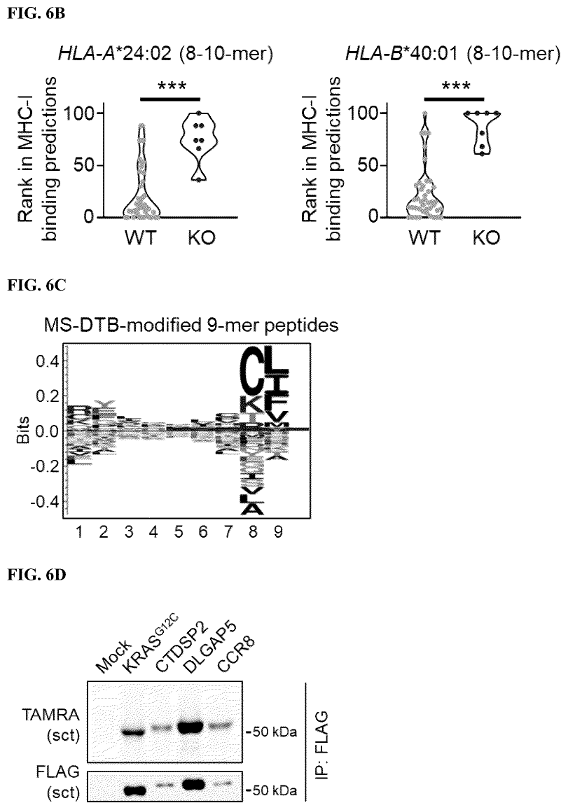

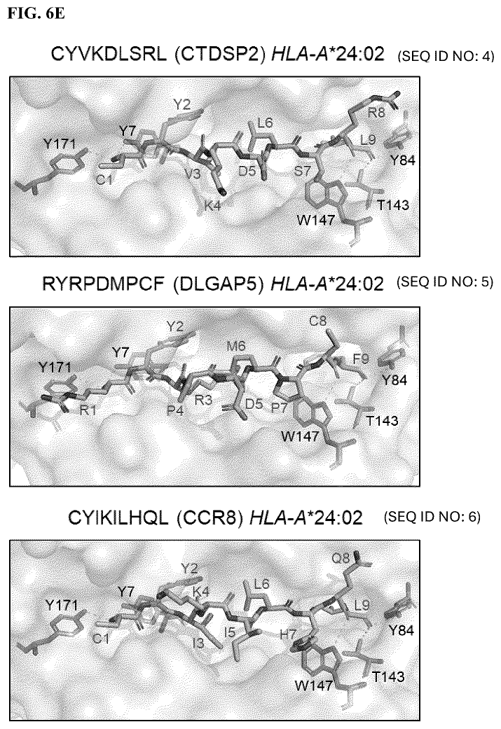

Next, this reactivity-based antigen profiling strategy was implemented in MT2 parental and HLA knockout cells to identify probe-modified reactive cysteine-containing MHC-I antigens (FIG. 6a). To evaluate the effectiveness of the probe in enriching MHC-I bound peptides, the MHC-I binding ranks predicted by IEDB for all probe-enriched 8-10-mer antigens were compared in MT2 parental and HLA knockout cells. The results indicated that probe-enriched peptides had significantly higher ranks for both HLA-A*24:02 and HLA-B*40:01 in HLA wildtype cells compared to HLA knockout cells, indicating that the MS-DTB probe is effective at enriching peptides bound to MHC-I (FIG. 6b). Nonetheless, the MS-DTB probe can still enrich peptides originating from other sources. To mitigate this effect, three filters were incorporated into the workflow: 1) restriction to 8-12-mer peptides in the search engine to exclude longer peptides from the pMHC-II complex; 2) utilization of a T cell epitope prediction algorithm to retain only top-ranked peptides for HLA-A*24:02 or HLA-B*40:01; and 3) selection of probe-modified peptides identified exclusively in MT2 parental cells, excluding peptides identified in HLA knockout cells. Using these filters, 95 MS-DTB-modified 8-12-mer peptides were identified in MT2 parental cells, with 39 falling within the top 20% ranking range using the T cell epitope prediction algorithm in IEDB (FIG. 15c). Motif analysis of 9-mer MS-DTB-modified peptides revealed a distribution pattern associated with the HLA alleles (HLA-A*24:01 and HLA-B*40:01) in MT2 cells (FIG. 6c). The preferred MS-DTB labeling site at position 8 is consistent with findings from immunopeptidomics (FIG. 3c, d). Conversely, in MT2 HLA knockout cells, among 43 MS-DTB-modified 8-12-mer peptides, only 3 are within the top 20% ranking range (FIG. 15c). Analysis of allele-specific MS-DTB modification of cysteines revealed distribution patterns similar to those observed in the immunopeptidomics study (FIG. 15d and FIG. 13d), further confirming the reliability of the findings regarding allele-specific probe modifications of antigen cysteines.

Next, probe-pulldown experiments were compared, focusing on 8-10-mer MS-DTB-modified peptides ranked within the top 20% ranking range, with 8-10-mer MS-DTB-modified peptides identified in immunopeptidomics. Of the 29 probe-modified peptides in probe-pulldown experiments, 9 were identified in both methods (FIG. 16a). The MHC-I binding ranks of these 9 peptides were compared with the 20 probe-modified peptides identified solely through probe-pulldown experiments. The result revealed that the 9 peptides identified by both methods have significantly higher MHC-I (HLA-A*24:02) binding ranks compared to the remaining 20 peptides (FIG. 16b). The relatively lower MHC-I (HLA-A*24:02) binding affinity of these 20 peptides may lead to their partial loss during sample preparation in immunopeptidomics. Conversely, in probe-pulldown experiments, the covalent interaction between the MS-DTB probe and these antigens may circumvent this limitation, allowing for the identification of probe-modified peptides.

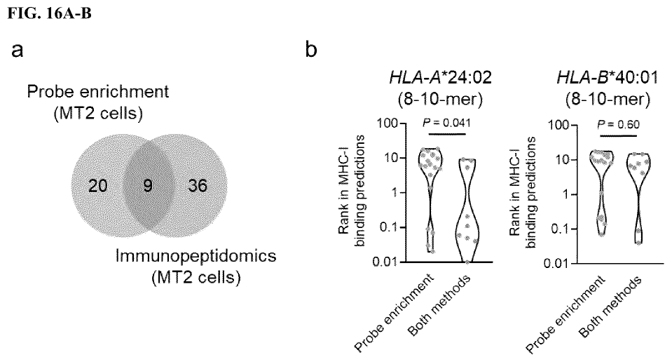

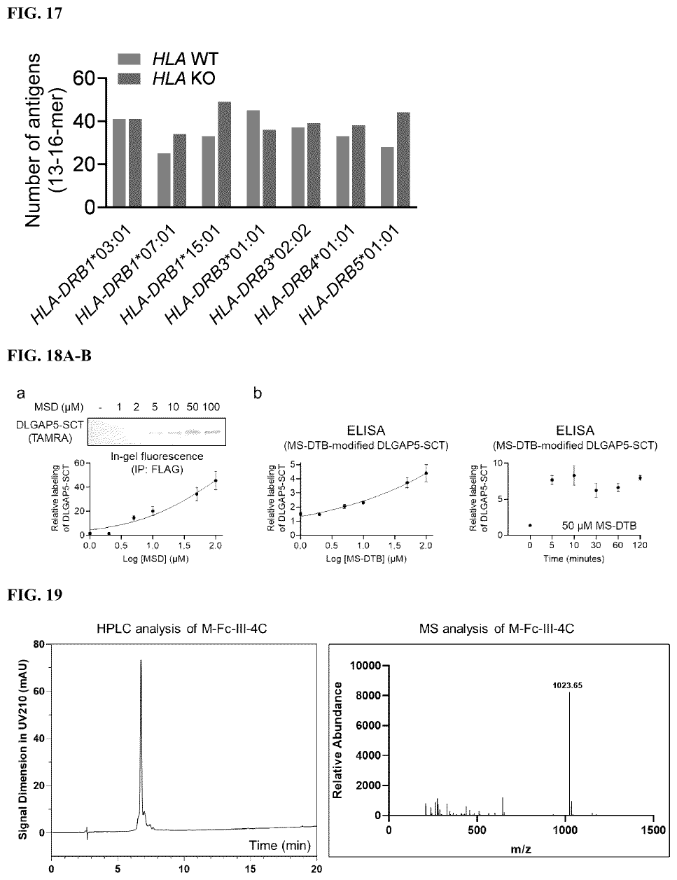

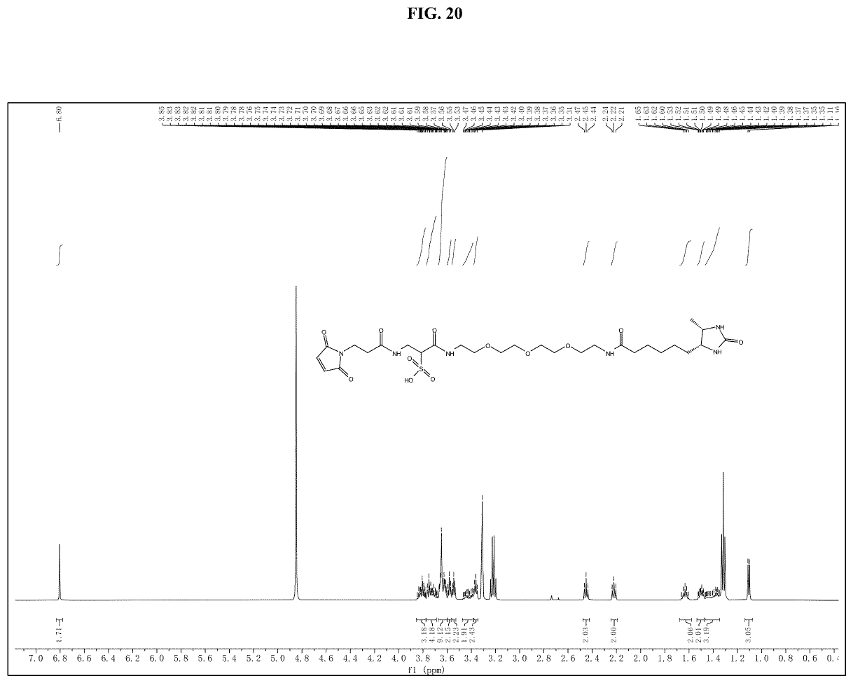

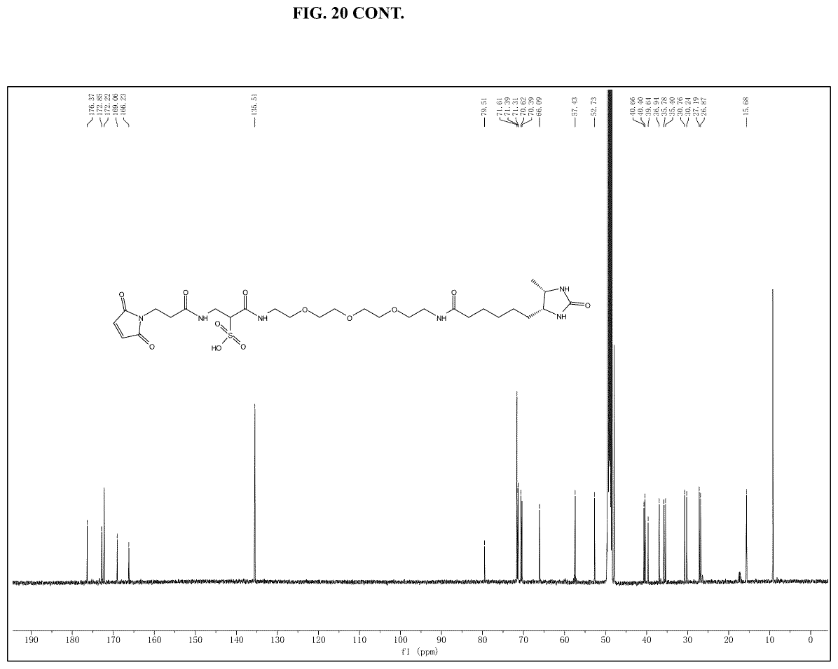

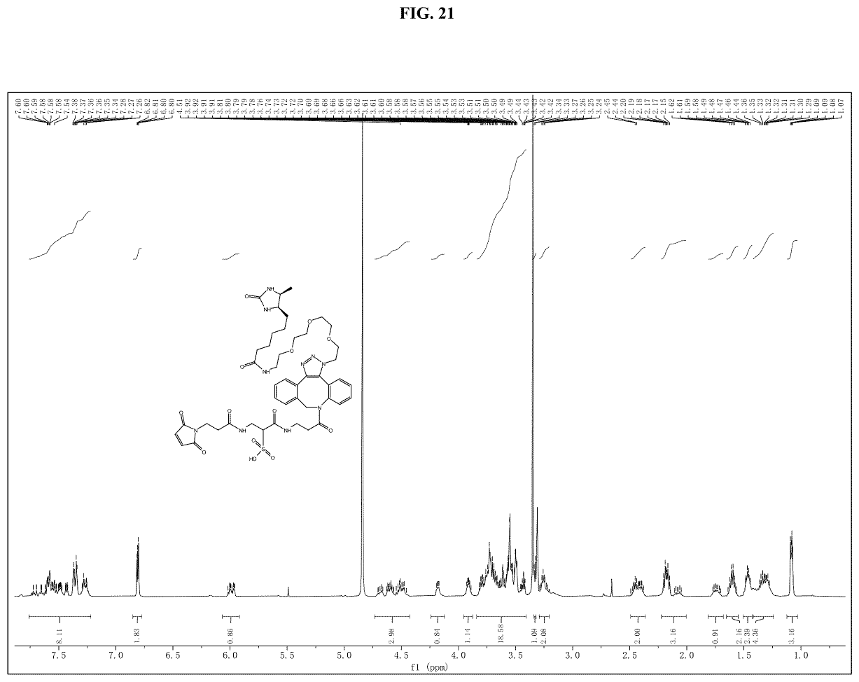

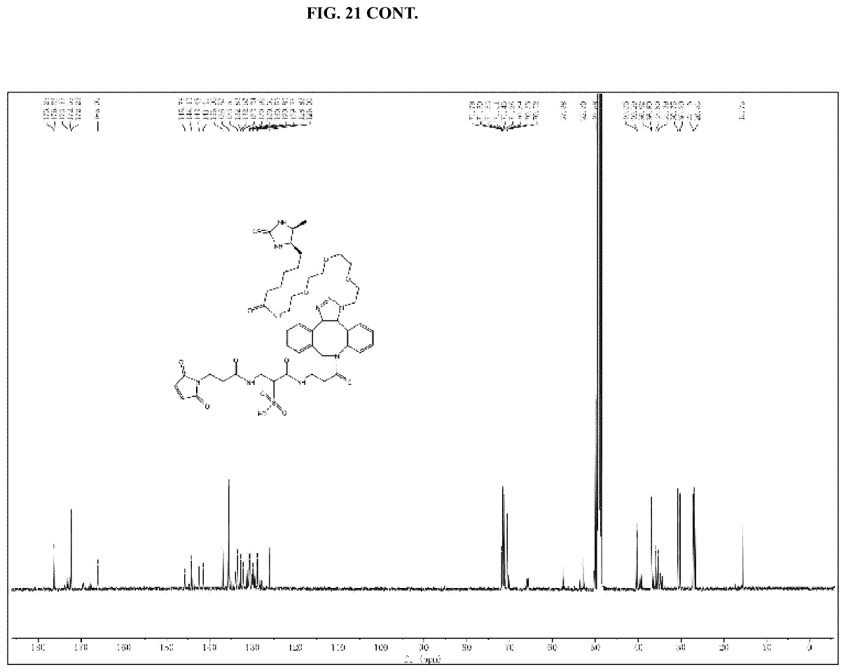

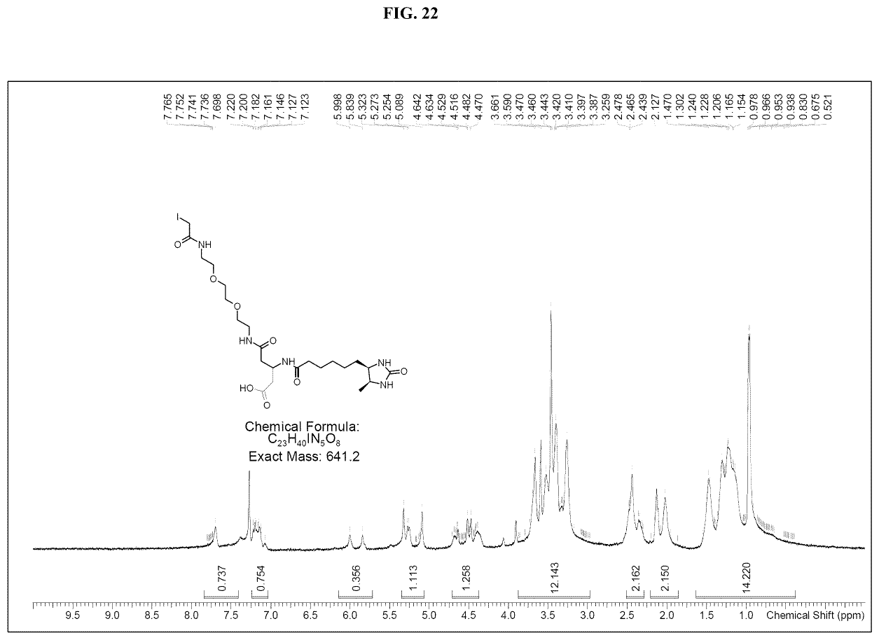

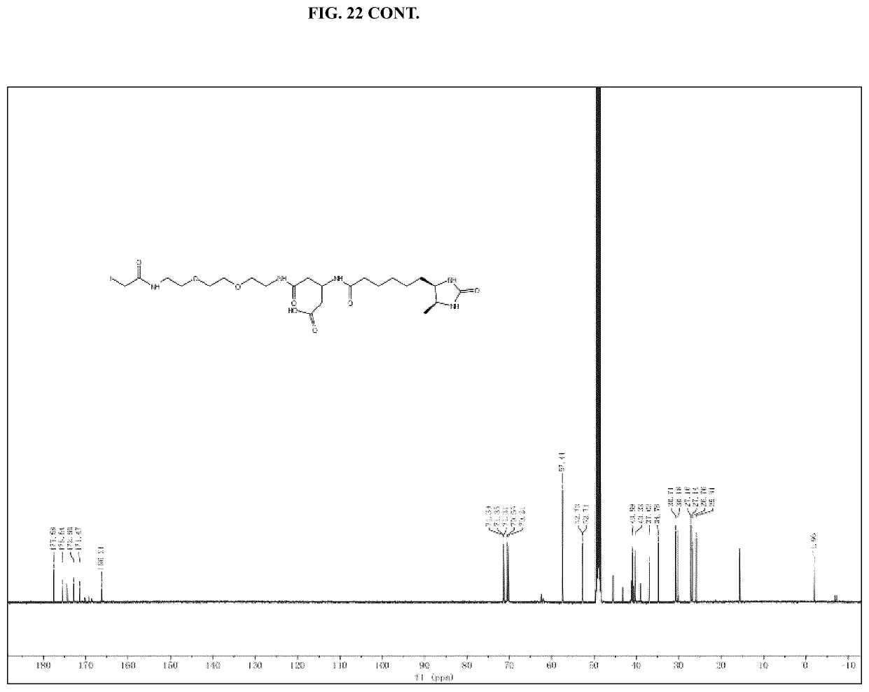

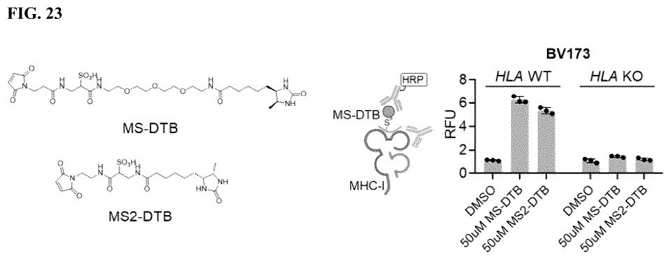

Since the MS-DTB probe may label MHC-II-associated peptides and cell surface proteins with unmodified cysteines, mass spectrometry data was analyzed to focus on 13-16-mer peptides within the top 20% ranks according to the IEDB MHC-II epitope prediction algorithm. The number of 13-16-mer peptides potentially associated with MHC-II is similar between MT2 parental and HLA knockout cells, which have comparable MHC-II protein expression (FIG. 10f and FIG. 17). Regarding cell surface proteins, the ABPP results from in-cell MS-DTB treatment (FIG. 1f) were re-analyzed. Among the 39 cysteines showing ≥15% engagement by the MS-DTB probe, only one cysteine, SORT_C86, is located in the extracellular domain of this transmembrane protein. Given the oxidizing extracellular environment, where most extracellular protein cysteines form disulfide bonds (Yi, M. C. & Khosla, C. Thiol-Disulfide Exchange Reactions in the Mammalian Extracellular Environment. Annu Rev Chem Biomol Eng 7, 197-222 (2016)), this data indicates that, at least in the HEK293T cell line, MS-DTB labeling of cell surface proteins is minimal.

Next, SCTs of HLA-A*24:02 were used to validate three MS-DTB-enriched antigens: CTDSP2 (CYVKDLSRL; SEQ ID NO: 4), DLGAP5 (RYRPDMPCF; SEQ ID NO: 5), and CCR8 (CYIKILHQL; SEQ ID NO: 6). In-gel fluorescence analysis demonstrated effective labeling of all three antigens by the MSD probe (FIG. 6d). Moreover, a modeling study using HLA-A*24:02 revealed that the anchor residues at positions 2 and 9 of all three antigen peptides fit into the B- and F-pockets, respectively (FIG. 6e). In the case of DLGAP5, where cysteine resides at position 8, the phenylalanine at anchoring position 9 is buried within the F-pocket and forms hydrogen bonds with Y84 and T143 in MHC-I. The carbonyl group of C8 is fixed with the NH in W147 through a hydrogen bond, which causes the cysteine at position 8 to be solvent exposed. As for CTDSP2 and CCR8 antigens, where cysteine is situated at position 1, the hydroxyl groups of Y7 and Y171 on MHC-I orient toward the A-pocket, forming hydrogen bonds with the N-terminal NH. Consequently, this arrangement results in the thiol of the cysteine residue being exposed to the solvent.

Using the DLGAP5-SCT, the probe was investigated through dose- and time-dependent labeling experiments. Both in-gel fluorescence and ELISA assays indicated that within the concentration range tested (1-100 μM), the probe exhibited increased labeling without reaching saturation (FIG. 18a, b). Since the cell-impermeable sulfonated maleimide probes are non-toxic (FIG. 9c), for future experiments, higher probe concentrations may provide broader coverage of labeled antigen cysteines. For labeling kinetics, results showed that 5 μM of MS-DTB achieved the highest labeling after 5-10 minutes (FIG. 18b), consistent with the rapid kinetics of the maleimide moiety in cysteine labeling (Ravasco, J., Faustino, H., Trindade, A. & Gois, P. M. P. Bioconjugation with Maleimides: A Useful Tool for Chemical Biology. Chemistry 25, 43-59 (2019)).

### Example 7

### Harnessing Cysteines in the Immunopeptidome to Induce Phagocytosis

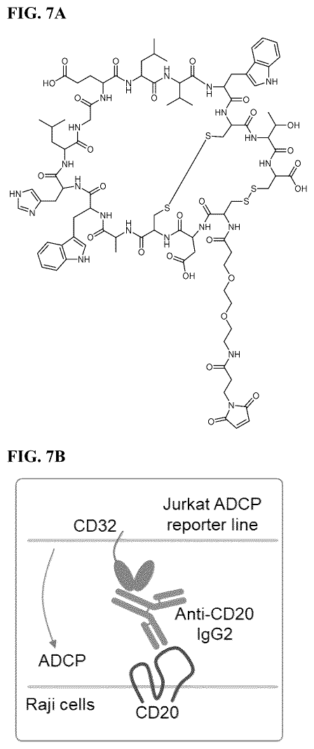

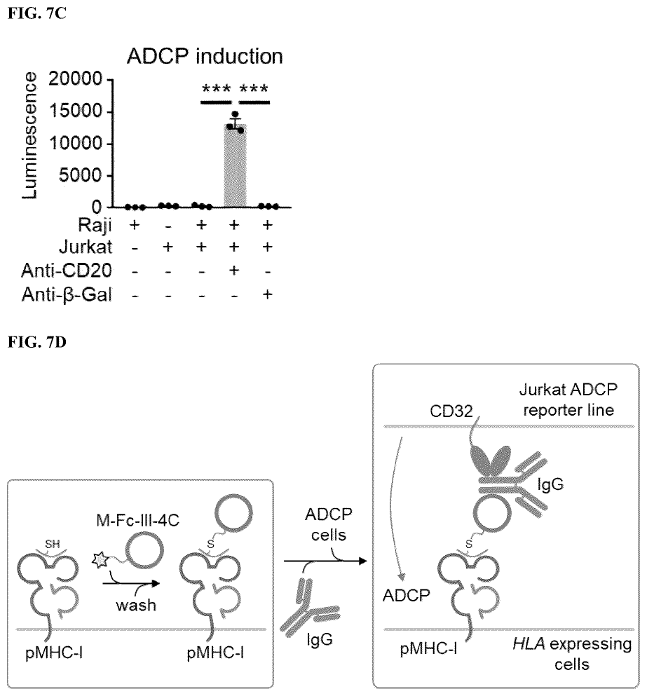

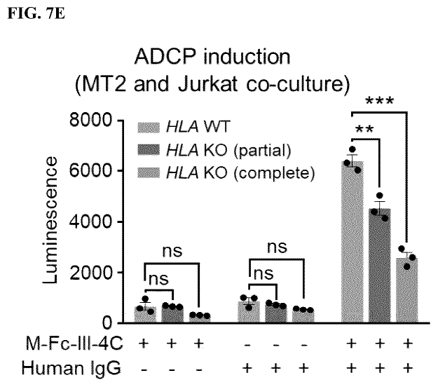

Experiments were conducted to demonstrate the application of this platform for targeting antigen cysteines to induce antibody-dependent cellular phagocytosis (ADCP), an immune mechanism involving Fc receptors, such as CD32, on effector cells that recognize and clear antibody-coated target cells41. A bifunctional molecule, M-Fc-III-4C (FIG. 7a), was synthesized by conjugating a maleimide moiety to Fc-III-4C, an Fc-binding cyclic peptide42, via a PEG linker. Fc-III-4C binds to a region of the Fc domain of human immunoglobulin G (IgG) that is distinct from the Fc receptor binding site (Sasaki, K. et al. Fc-binding antibody-recruiting molecules exploit endogenous antibodies for anti-tumor immune responses. Chem Sci 11, 3208-3214 (2020), making it a suitable component for Antibody Recruiting Molecules (ARMs) designed to recruit immune cells such as macrophages and natural killer (NK) cells (McEnaney, P. J., Parker, C. G., Zhang, A. X. & Spiegel, D. A. Antibody-recruiting molecules: an emerging paradigm for engaging immune function in treating human disease. ACS Chem Biol 7, 1139-1151 (2012)). To evaluate M-Fc-III-4C, a Jurkat-Luc NFAT-CD32 cell model, which features an NFAT-inducible luciferase reporter gene activated by the Fc receptor CD32 upon encountering antibody-coated cells, was used (FIG. 7b, c). MT2 parental, HLA partial knockout, and HLA complete knockout cells were treated with M-Fc-III-4C, unbound compound was washed away, and the labeled cells were co-cultured with Jurkat-Luc NFAT-CD32 cells in the presence of human IgG (FIG. 7d). ADCP was observed with MT2 parental cells, whereas a significant decrease in ADCP was noted in MT2 HLA knockout cells (FIG. 7e). These results indicate that the ADCP induced by M-Fc-III-4C is likely partially mediated by reactive cysteines on MHC-I-bound antigens.

### Example 8

### T-Cell Recruitment

Experiments are conducted to assay T-cell recruitment. The cysteine-reactive probe is conjugated to a T cell-recruiting moiety, such as a CD3-binding single-chain variable fragment (scFv), through a linker. The resulting molecule is used to facilitate T cell recruitment by simultaneously binding to antigen-presenting cells and CD3-expressing T cells.

### Methods

Reagents. The anti-FLAG HRP antibody (clone M2, cat #: A8592) and anti-FLAG affinity gel (clone M2, cat #: A2220) were purchased from Sigma-Aldrich. The anti-β-Actin antibody (clone #: C4, cat #: sc-47778) was purchased from Santa Cruz Biotechnology. Streptavidin-HRP (cat #: 3999), anti-GAPDH (clone 14C10, cat #: 3683), anti-MHC Class I (clone EMR8-5, cat #: 88274) and anti-PSMB9 (clone E7JIL, cat #: 87667) antibodies were purchased from Cell Signaling Technology. The anti-hCD20-hIgG2 (clone Rituximab: anti-hCD20-hIgG2, kappa, cat #: hcd20-mab2) and anti-β-Gal-hIgG2 (monoclonal, cat #: bga1-mab2) antibodies were purchased from InvivoGen. Puromycin (cat #: ant-pr-1) was purchased from InvivoGen. InVivoMAb anti-human MHC Class I (HLA-A, HLA-B, HLA-C) (clone W6/32, cat #: BE0079) for ELISA assay was purchased from Bio X Cell. Ultra-LEAF anti-human HLA-A,B,C antibody (clone W6/32, cat #: 311448) for immunopeptidomics was purchased from BioLegend. Human IgG isotype (cat #: 31154) was purchased from Thermo Scientific. Polyethylenimine (PEI, MW 40,000, cat #: 24765-1) was purchased from Polysciences, Inc. Tetramethylrhodamine (TAMRA) azide (cat #: T10182), enzyme-linked chemiluminescence (ECL) (cat #: 32106) western blotting detection reagents, QuantaBlu fluorogenic peroxidase substrate kit (cat #: 15169), Streptavidin agarose (cat #: 20349), Streptavidin-FITC (cat #: 11-4317-87), HLA-A,B,C FITC antibody (clone W6/32, cat #MA5-44095), beta-2-microglobulin HRP (clone B2M-01, cat #: MA1-19679), and Tandem Mass Tag (TMT) isobaric label reagent (cat #: 90066 for TMTsixplex and cat #90406 for TMT10plex) were purchased from Thermo Scientific. FuGene 6 (cat #: E2692) transfection reagent and sequencing grade modified trypsin (cat #: V5111) were purchased from Promega. Cas9 endonuclease was purchased from Integrated DNA Technologies. pMHC-I-HLA-A2-KRAS-G12C was purchased from ProImmune. Maleimide-sulfonate-dibenzocyclooctyne (MSD), Maleimide-dibenzocyclooctyne (MD), Maleimide-sulfonate-PEG4-dibenzocyclooctyne (MSD4), and N-hydroxysuccinimide (NHS)-sulfonate-biotin were purchased from BroadPharm.

Cell lines. HEK293T and MDA-MB-231 cells were obtained from ATCC. BV173 cells were obtained from CLS Cell Lines Service. MT2 cells were obtained from Thermo Scientific. Jurkat-Lucia NFAT-CD32 cells and Raji cells were obtained from InvivoGen. HEK293T and MDA-MB-231 cells were cultured in Dulbecco's Modified Eagle Medium (DMEM, Corning) with 10% (v/v) fetal bovine serum (FBS, Omega Scientific) and L-glutamine (2 mM, Gibco). BV173, MT2 and Raji cells were cultured in RPMI 1640 (Corning) with 10% (v/v) FBS (Omega Scientific) and L-glutamine (2 mM, Gibco). Jurkat-Lucia NFAT-CD32 cells were cultured in Iscove's Modification of DMEM (Corning) with 10% (v/v) FBS (Omega Scientific). All the cell lines were tested negative for mycoplasma contamination.

Generation of CRISPR-Cas9-mediated HLA knockout cells. BV173, MT2 and MDA-MB-231 cells with HLA-A,B,C CRISPR-Cas9 knockout were generated through electroporation of Cas9-sgRNA ribonucleoprotein (RNP) complex using 4D-Nucleofector (Lonza Bioscience). Three sgRNAs targeting HLA gene (HLA sgRNA #1: CGGCTACTACAACCAGAGCG (SEQ ID NO:7); HLA sgRNA #2: AGATCACACTGACCTGGCAG (SEQ ID NO:8); HLA sgRNA #3: AGGTCAGTGTGATCTCCGCA (SEQ ID NO:9)) were mixed for the electroporation.

Cloning and mutagenesis. Human KRAS4A-G12C cDNAs with N-terminal FLAG tag and all single-chain trimers were purchased as gene block from Integrated DNA Technologies and cloned into pCDH-CMV-MCS-EF1-Puro vector via NehI and BamHI sites. pp65 mutants were generated using Q5 site-directed mutagenesis kit (New England Biolabs).

Generation of KRAS4A-G12C stably expressed cells. Lentivirus containing FLAG-KRAS4A-G12C were generated by co-transfection of FLAG-KRAS4A-G12C, psPAX2 and pMD2. G into HEK293T cells using FuGene 6 transfection reagent. Medium containing lentiviral particles were collected 48 hours post transfection, filtered with 0.45 μM Millex-HV sterile syringe filter unit (MilliporeSigma), and used to transduce BV173 cells in the presence of 10 μg/mL polybrene. 48 hours post transduction, puromycin (2 μg/mL) was added and incubated with the cells for 7 days.

Cell lysis and Western blot. Cells were lysed utilizing radioimmunoprecipitation assay (RIPA) lysis buffer (Thermo Scientific) comprising 25 mM Tris-HCl, pH 7.6, 150 mM NaCl, 1% Nonidet P40 (NP-40), 1% sodium deoxycholate, and 0.1% sodium dodecyl sulfate (SDS). Before usage, the lysis buffer was supplemented with the complete protease inhibitor cocktail (Roche). The cell suspension underwent sonication through 5 cycles at 40% power for 4 pulses each. Subsequent to sonication, the resultant mixture underwent centrifugation at 16,000 g for 10 minutes at 4° C. to acquire the supernatant. The protein concentration in the supernatant was determined employing the DC assay (Bio-Rad). The protein lysate was combined with Laemmli sample buffer (Bio-Rad) and heated at 95° C. for 5 minutes. Proteins were analyzed using 4-20% Novex Tris-Glycine mini gels (Invitrogen), followed by transfer onto a 0.2 μM polyvinylidene fluoride (PVDF) membrane (Bio-Rad). The PVDF membrane was incubated with 5% non-fat milk in Tris-buffered saline with Tween 20 (TBST) buffer (0.1% Tween 20, 20 mM Tris-HCl at pH 7.6, and 150 mM NaCl) for 1 hour at room temperature. Primary antibodies were diluted in 5% non-fat milk in TBST buffer and incubated with the membrane. Incubation durations were 1 hour at room temperature for FLAG and β-actin, and overnight at 4° C. for others. Following primary antibody incubation, the membrane underwent three washes with TBST buffer and was then incubated with a secondary antibody (diluted 1:5000 in 5% non-fat milk in TBST) for 1 hour at room temperature. After three additional washes with TBST buffer, the chemiluminescence signal on the membrane was developed using ECL Western blotting detection reagent, and the resultant signal was captured using ChemiDoc MP (Bio-Rad).

Immunoprecipitations. Cells were lysed in NP-40 lysis buffer (25 mM Tris-HCl, pH 7.4, 150 mM NaCl, 10% glycerol, 1% NP-40) supplemented with complete protease inhibitor cocktail. The cell suspension was incubated on ice for 10 minutes. Following this, the mixture was centrifuged at 16,000 g for 10 minutes at 4° C., and the resulting supernatant was collected for use in immunoprecipitation. For immunoprecipitation, FLAG affinity gel (25 μL slurry per sample) was added to the protein lysates and rotated at 4° C. for 2 hours. The affinity gel was then washed four times with immunoprecipitation washing buffer comprising 0.2% NP-40, 25 mM Tris-HCl at pH 7.4, and 150 mM NaCl. Subsequently, the affinity gel was mixed with Laemmli sample buffer and heated at 95° C. for 10 minutes. The resulting supernatant, containing the eluted proteins, was collected and utilized for subsequent western blot analysis.

Global proteomics. For global proteomics comparing MT2 WT and HLA partial KO cells, two biological replicates were used for each group. For comparisons between MT2 WT and HLA complete KO cells, MDA-MB-231 WT and HLA KO cells, and BV173 WT and HLA KO cells, three biological replicates were used for each group. Cells were lysed in 100 μL of PBS using sonication (10 pulses at 40% intensity, 3 rounds). Protein concentration was determined via a DC assay. Next, 100 μg of proteins in 100 μL of lysis buffer were denatured with 8 M urea. For reduction, 5 μL of 200 mM dithiothreitol (DTT) stock solution in water was added, and the mixture was heated to 65° C. for 15 minutes. Alkylation was achieved by adding 5 μL of 400 mM iodoacetamide stock solution in water and incubating in the dark at 37° C. for 30 minutes. Proteins were then precipitated by adding 600 μL of methanol, 200 μL of chloroform, and 500 μL of water. After precipitation, protein pellets were washed with 1 mL of methanol. The resulting protein pellets were solubilized in 160 μL of 4-(2-hydroxyethyl) piperazine-1-propanesulfonic acid (EPPS) buffer (200 mM). Subsequently, 2 μg of LysC was added to each sample, and digestion was carried out at 37° C. for 2 hours. This was followed by the addition of 5 μg of trypsin to each sample for another round of digestion, allowed to proceed at 37° C. for 12 hours. For TMT labeling, 12.5 μg of resulting peptides in 35 μL of EPPS buffer were utilized. To each sample, 9 μL of acetonitrile was added, followed by TMT tags (3 μL per sample). The samples were then incubated at room temperature for 1 hour. The TMT labeling reaction was quenched by adding 6 μL of a 5% hydroxylamine solution, followed by the addition of 2.5 μL of formic acid. The samples were pooled and separated into 12 distinct fractions using the Thermo Vanquish Ultra High-Performance Liquid Chromatography (UHPLC) fractionator. These fractions were analyzed on an Orbitrap Eclipse Tribrid mass spectrometer coupled with a Vanquish Neo UHPLC system. Peptides were injected onto an EASY-Spray HPLC column (C18, 2 μm particle size, 75 μm inner diameter, 250 mm length) and eluted at a flow rate of 0.25 μL/min, following a gradient: 5% buffer B (80% acetonitrile with 0.1% formic acid) in buffer A (water with 0.1% formic acid) from 0 to 15 minutes, 5% to 45% buffer B from 15 to 155 minutes, and 45% to 100% buffer B from 155 to 180 minutes. The parameters for the MS1 scan are: resolution 120,000, m/z range 375-1600, RF lens 30%, standard automatic gain control (AGC) target and auto maximum injection time. In the MS2 analysis, precursor ions were quadrupole-isolated (isolation window 0.7) and then subjected to higher-energy collisional dissociation (HCD) collision in the ion trap (standard AGC, collision energy 30%, maximum injection time 35 ms). Following each MS2 spectrum, synchronous precursor selection (SPS) enabled the selection of 10 MS2 fragment ions for MS3 analysis. These MS3 precursors were fragmented by HCD and analyzed using the Orbitrap (collision energy 55%, AGC 250%, maximum injection time 200 ms, resolution 60,000). The RAW data was analyzed using Proteome Discoverer 2.5. Cysteine residues were searched with a static modification for carbamidomethylation (+57.0215). Methionine residues were searched with a dynamic modification for oxidation (+15.9949). Lysine residues and peptide N-termini were searched with a static modification for TMT labeling (+229.1629). MS3 quantification was performed with 6-plex or 10-plex TMT analysis parameters (6-plex: m/z 126.127725, 127.12476, 128.134433, 129.131468, 130.141141 and 131.138176; 10-plex: m/z 126.127726, 127.124761, 127.131081, 128.128116, 128.134436, 129.131471, 129.13779, 130.134825, 130.141145 and 131.13818) with a mass tolerance of 30 ppm. Protein relative abundance was calculated based on the corresponding MS3 intensity.

Cysteine-directed ABPP. For cysteine-directed ABPP that measures proteome-wide cysteine engagement by MS-DTB in cell lysates or live cells, and cysteine-directed ABPP that identifies cysteine-containing peptides directly enriched by IA-DTB, MS-DTB, or MSD-DTB, two biological replicates were used for each group. Cells were lysed in PBS via sonication (10 pulses at 40% intensity, 3 rounds). The protein concentration was determined using a DC assay and adjusted to 1 mg/mL. Next, 500 μL of lysates were labeled with 100 μM IA-DTB, DBIA, MS-DTB or MSD-DTB at room temperature for 1 hour. Protein precipitation was achieved by adding 500 μL of methanol and 100 μL of chloroform, followed by a methanol wash (1 mL). The resulting protein pellets were denatured using 90 μL of 9 M urea and 10 mM DTT in 50 mM tetramethylammonium bicarbonate. Alkylation was carried out using 50 mM iodoacetamide at 37° C. for 30 minutes. Subsequently, 350 μL of 50 mM tetramethylammonium bicarbonate was added to each sample, followed by the addition of 2 μg of trypsin. Digestion was allowed to proceed at 37° C. for 12 hours. Next, 50 μL of streptavidin-agarose beads were added to each sample, and the mixture was rotated at room temperature for 2 hours. The beads were washed three times with 1 mL of washing buffer consisting of 0.2% NP-40, 25 mM Tris-HCl pH 7.4, and 150 mM NaCl, followed by three washes with 1 mL of PBS, and two washes with 1 mL of water. Peptides were eluted using 300 μL of 50% acetonitrile containing 0.1% formic acid. The eluted peptides were subsequently dried using a SpeedVac vacuum concentrator. The subsequent steps of TMT labeling and LC-MS analysis were carried out following the methodology described in global proteomics. During RAW data analysis, cysteine residues were searched with a dynamic modification for carbamidomethylation (+57.0215), IA-DTB (455.2744), DBIA (296.1848), MS-DTB (690.2894), hydrolyzed MS-DTB (708.3000), MSD-DTB (992.4062) or hydrolyzed MSD-DTB (1010.4168).

Immunopeptidomics. For immunopeptidomics, two biological replicates were used for each group. 2×10⁸ BV173 or MT2 cells were lysed using 3 mL of lysis buffer (0.5% NP-40, 50 mM Tris pH 8.0, 150 mM NaCl, 1 mM ethylenediaminetetraacetic acid (EDTA), and protease inhibitor cocktail) by rotating at 4° C. for 30 minutes. Following centrifugation at 18,000 g for 10 minutes, the supernatant was collected for enrichment using an anti-MHC antibody (W6/32, BioLegend) conjugated to Affi-gel 10 matrix (Bio-Rad, 2 mg of antibody per 100 μL of slurry per sample). Enrichment occurred over 4 hours of rotation at 4° C., followed by transfer to a Bio-spin column (Bio-Rad) for washing with 3×1 mL of lysis buffer, wash buffer 1 (50 mM Tris pH 8, 150 mM NaCl), wash buffer 2 (50 mM Tris pH 8, 400 mM NaCl), and wash buffer 3 (50 mM Tris pH 8). MHC-conjugated peptides were subsequently eluted using 1 mL of 1% trifluoroacetic acid in water. Peptide samples were desalted using a Sep-Pak C18 cartridge (Waters), dried via speedavac, and analyzed using an Orbitrap Eclipse Tribrid mass spectrometer coupled with a Vanquish Neo UHPLC system. The subsequent step LC-MS analysis was carried out following the methodology described in global proteomics. Motif analysis of peptides was performed using the Seq2Logo method⁴⁸.

Reactivity-based antigen profiling. For probe enrichment experiments, two biological replicates were used for each group. 10⁸ cells were treated with 5 μM of MS-DTB for 30 minutes. After treatment, the cells were washed twice with PBS and then harvested. Subsequently, the cells were lysed in 5 mL of lysis buffer containing 2M urea and 0.2% NP-40 in PBS using sonication (10 pulses at 40% intensity, 3 rounds). Following centrifugation at 18,000 g for 10 minutes, the supernatant was collected for enrichment using streptavidin agarose beads. The mixture was rotated at room temperature for 2 hours. The beads were then washed three times with 1 mL of washing buffer (0.2% NP-40, 25 mM Tris-HCl pH 7.4, and 150 mM NaCl), followed by three washes with 1 mL of wash buffer 1 (50 mM Tris pH 8, 150 mM NaCl), wash buffer 2 (50 mM Tris pH 8, 400 mM NaCl), PBS, and water. Peptides were eluted using 300 μL of 50% acetonitrile containing 0.1% formic acid. The eluted peptides were subsequently dried using a SpeedVac vacuum concentrator and desalted using a Sep-Pak C18 cartridge. The peptides were analyzed using an Orbitrap Eclipse Tribrid mass spectrometer coupled with a Vanquish Neo UHPLC system. The subsequent step LC-MS analysis was carried out following the methodology described in global proteomics.

Modeling study. The crystal structures of HLA-A*02:01 (2X4R) and HLA-A*24:02 (2BCK) from Protein Data Bank (X-ray structures with a resolution finer than 3.5 Å), KRASG12C neoantigen (KLVVVGACGV; SEQ ID NO:3) and three MS-DTB-enriched antigens: CTDSP2 (CYVKDLSRL; SEQ ID NO:4), DLGAP5 (RYRPDMPCF; SEQ ID NO:5), and CCR8 (CYIKILHQL; SEQ ID NO:6) were used for the modeling study. MHC-Fine, a refined AlphaFold model, was used for MHC-peptide complex prediction (Glukhov, E. et al. MHC-Fine: Fine-tuned AlphaFold for Precise MHC-Peptide Complex Prediction. 2023.2011.2029.569310 (2023)). The final PDB files of the MHC-peptide complex were generated by running the inference code and datasets (https://bitbucket.org/abc-group/mhc-fine/src/main/). The figures were generated by PyMOL software. For each MHC protein, only the α1 and α2 domains were used.

The FASTA sequence of HLA-A*02:01:
(SEQ ID NO: 1)
GSHSMRYFFTSVSRPGRGEPRFIAVGYVDDTQFVRFDSDAASQRM
EPRAPWIEQEGPEYWDGETRKVKAHSQTHRVDLGTLRGYYNQSEA
GSHTVQRMYGCDVGSDWRFLRGYHQYAYDGKDYIALKEDLRSWTA
ADMAAQTTKHKWEAAHVAEQLRAYLEGTCVEWLRRYLENGKETLQ
RT
The FASTA sequence of HLA-A*24:02:
(SEQ ID NO: 2)
GSHSMRYFSTSVSRPGRGEPRFIAVGYVDDTQFVRFDSDAASQRM
EPRAPWIEQEGPEYWDEETGKVKAHSQTDRENLRIALRYYNQSEA
GSHTLQMMFGCDVGSDGRFLRGYHQYAYDGKDYIALKEDLRSWTA
ADMAAQITKRKWEAAHVAEQQRAYLEGTCVDGLRRYLENGKETLQ
RT

Cell surface probe labeling by flow cytometry. Cells were seeded in non-treated 6-well plates and treated with 50 μM of reactivity probes for 30 minutes. Following treatment, cells were rinsed with PBS and suspended in flow cytometry buffer (1 mM EDTA, 25 mM HEPES pH 7.0, 1% FBS in PBS) within Eppendorf tubes. Subsequently, Streptavidin-FITC or HLA-A,B,C-FITC antibody (diluted 1:50) was added, and the cells were rotated at room temperature for 30 minutes. Afterward, the cells were washed with PBS and resuspended in flow cytometry buffer. FITC fluorescence on the cell surface was quantified using a BD LSRFortessa Cell Analyzer, and the resulting data were analyzed utilizing FlowJo software.

LC-MS analysis of probe-peptide adduct. Peptides were synthesized by GenScript. 100 UM peptide and 100 μM compound were incubated in water for 30 minutes and analyzed by Thermo Vanquish UHPLC coupled to ISQ EC Single Quadrupole Mass Spectrometer. Peptide and probe-peptide adduct were separated on Gemini C18 column (Phenomenex, 5 μm, 50×4.6 mm) at a flow rate of 1 mL/min, following the gradient: 0 to 95% buffer C (acetonitrile with 0.1% formic acid) in buffer A (water with 0.1% formic acid) from 0 to 13 minutes, and 95% buffer C in buffer A from 13 to 20 minutes. 210 nm wavelength was used to monitor the peaks. A full scan from m/z 200-1250 with positive mode was used to analyze unmodified peptide and probe-peptide adduct. Two biological replicates were used for each group.

| ELISA assay. Nunc MaxiSorp 384-well plates (black) were coated overnight with 50 μL of the anti-heavy chain antibody W6/32 at a concentration of 5 μg/mL in PBS. Following coating, the plates were washed twice with PBS (100 μL) and blocked with 3% bovine serum albumin (BSA) in PBS (120 μL) at room temperature for 1 hour. Subsequently, the plates were washed three times with 0.05% Tween-20 in PBS (PBST) (100 μL each wash). BV173, MT2 parental, and HLA knockout cells were treated with 50 μM of MS-DTB for 30 minutes, then harvested and lysed in NP-40 lysis buffer with protease inhibitor cocktail. The protein concentration was adjusted to 1 mg/mL, and 50 μL of total lysates were added to each well. Plates were incubated at 4° C. for 4 hours, followed by three washes with 1% BSA in PBS (100 μL each wash). Next, 50 μL of either 1 μg/mL anti-beta-2-microglobulin HRP conjugate solution or Streptavidin-HRP (diluted 1:1000) in 1% BSA PBS was added to each well. Plates were incubated with shaking at room temperature for 1 hour. The plates were washed three times with PBST and three times with PBS (100 μL each wash). 50 μL of the HRP substrate QuantaBlu was added, and fluorescence was measured using the CLARIOstar Plus microplate reader (BMG Labtech).

In-gel fluorescence. HEK293T cells were transfected with SCTs using PEI transfection reagent. Following a 24-hour incubation, the cells were treated with 5 μM of MSD for 30 minutes. Subsequently, cells were harvested by centrifugation at 500 g for 5 minutes and then lysed in NP-40 lysis buffer containing protease inhibitor cocktail. The resulting lysate was subjected to immunoprecipitation with anti-Flag affinity gel at 4° C. for 2 hours. The affinity gel was washed three times with immunoprecipitation washing buffer and re-suspended in 18 μL of PBS. For the click chemistry reaction, the following reagents were added: 0.8 μL of 1.5 mM TAMRA azide solution in DMSO, 1.2 μL of 10 mM Tris(benzyltriazolylmethyl)amine solution in 4:1 tBuOH:DMSO, 1 μL of 40 mM CuSO₄ solution in H₂O, and 1 μL of 40 mM Tris(2-carboxyethyl) phosphine solution in H₂O. The reaction proceeded at room temperature for 1 hour. Subsequently, Laemmli sample buffer was added and heated at 95° C. for 10 minutes. Following centrifugation at 15,000 g for 2 minutes, the supernatant was collected, and the samples were resolved by 4-20% Novex Tris-Glycine mini gels. In-gel fluorescence signals were recorded using the ChemiDoc MP system.

Cell viability assay. Cells were plated in a 96-well clear bottom white plate (Corning) at a density of 5,000 cells per well in 100 μL of DMEM medium and incubated for 24 hours. Subsequently, the cells were treated with varying concentrations of compounds in 100 μL of DMEM medium for additional 72 hours. Following treatment, 50 μL of Cell Titer Glo reagent (Promega) was added to each well and incubated for 10 minutes at room temperature. Luminescence was measured using CLARIOstar Plus microplate reader (BMG Labtech).

Antibody-dependent cellular phagocytosis assay. For the assay with Raji cells, 90 μL of Raji cells in RPMI (1.1×10⁵ cells per well) were added in 96-well plate in the presence of 20 μL anti-hCD20-hIgG2 (5 μg/mL) or anti-β-Gal-hIgG2 (10 μg/mL). After 1 hour incubation, Raji cells were co-cultured with Jurkat-Luc NFAT-CD32 cells (2.2×10⁵ cells per well). The mixture was then incubated at 37° C. for 8 hours. 20 μL of the cultured supernatant was transferred to a 96-well white plate and mixed with 50 μL of QUANTI-Luc Lucia 4 (InvivoGen) per well. The luciferase activity was measured using CLARIOstar Plus microplate reader. For the assay targeting reactive cysteines of MHC-I-bound antigens in MT2 cells, MT2 wildtype and HLA knockout cells were treated with DMSO or M-Fc-III-4C (10 μM) in serum free RPMI medium for 30 minutes. After washing with PBS to remove free compound, 90 μL of cell suspension (1.1×10⁵ cells per well) were added in 96-well plate in the presence of 20 μL of human IgG isotype control (5 μg/mL). MT2 cells were subsequently co-cultured with Jurkat-Luc NFAT-CD32 cells (2.2×10⁵ cells per well). The mixture was incubated at 37° C. for 8 hours. 20 μL of the cultured supernatant was transferred to a 96-well white plate and mixed with 50 μL of QUANTI-Luc Lucia 4 (InvivoGen) per well. The luciferase activity was measured using CLARIOstar Plus microplate reader.

Statistical analysis. Quantitative data were depicted using scatter plots, displaying the mean accompanied by the standard error of the mean (SEM) represented as error bars. Differences between two groups were assessed using an unpaired two-tailed Student's t-test. Significance levels were denoted as follows: *P<0.05, **P<0.01, ***P<0.001, and ns, not significant. Statistical significance was defined for P values <0.05.

Data Availability. The mass spectrometry proteomics data have been deposited to the ProteomeXchange Consortium via the PRIDE (Perez-Riverol, Y. et al. The PRIDE database resources in 2022: a hub for mass spectrometry-based proteomics evidences. Nucleic Acids Res 50, D543-D552 (2022)) partner repository with the dataset identifier PXD054678 (https://proteomecentral.proteomexchange.org/cgi/GetDataset?ID=PXD054678). The protein structures were retrieved from the Protein Data Bank with the accession codes: 2X4R (https://doi.org/10.2210/pdb2x4r/pdb); 2BCK (https://doi.org/10.2210/pdb2bck/pdb).

Code Availability. The code for the MHC-peptide complex modeling study is available on Bitbucket: https://bitbucket.org/abc-group/mhc-fine/src/main/.

### Synthetic Procedures

### Synthesis of maleimide-sulfonate-DTB (MS-DTB)

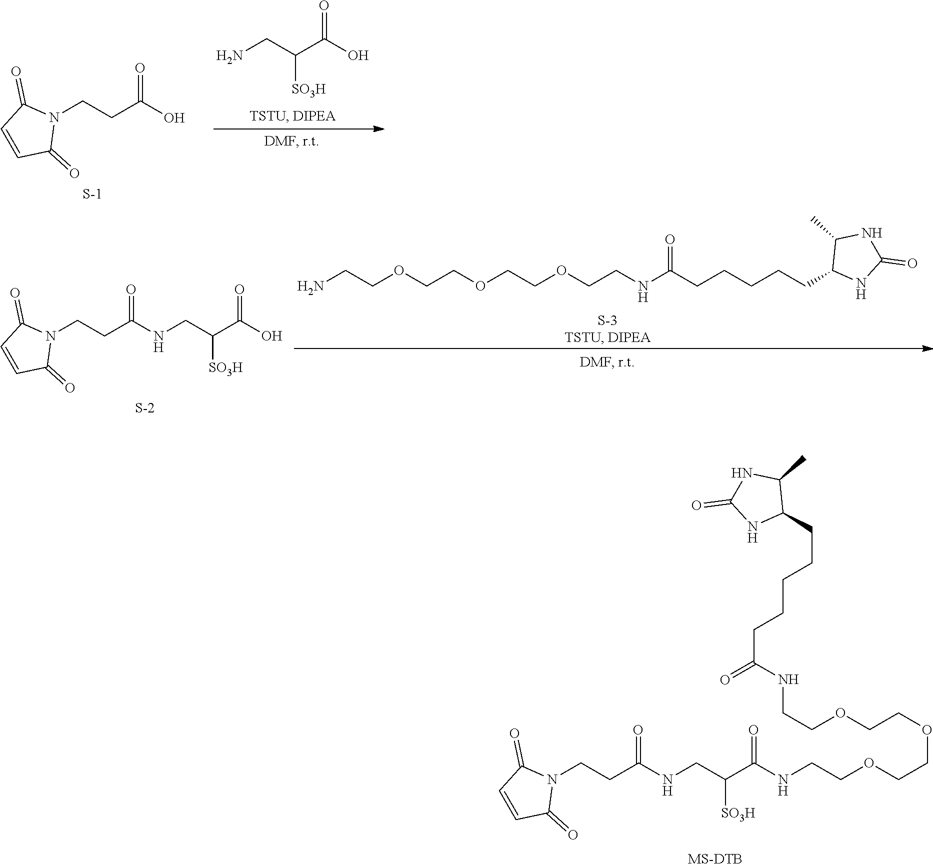

Step 1:3-Maleimidopropionic acid S-1 (50 mg, 0.30 mmol, 1.0 eq) and TSTU (117 mg, 0.39 mmol, 1.3 eq) was dissolved in DMF (1 mL). Then DIPEA (149 μL, 0.90 mmol, 3.0 eq) was added and stirred for 1 h at room temperature. After the formation of succinate, 3-amino-2-sulfopropanoic acid (75 mg, 0.45 mmol, 1.5 eq) was added and stirred overnight. Upon completion, the reaction was concentrated, and the resulting residue was purified by flash chromatography to provide S-2 as a colorless oil (61 mg, 0.19 mmol, 63%).

¹H NMR (400 MHz, CD₃OD) δ 6.79 (s, 2H), 3.79-3.70 (m, 5H), 2.46 (t, J=6.8 Hz, 2H).

Step 2: S-3 was prepared as reported previously (Zhang, T. et al. Covalent targeting of remote cysteine residues to develop CDK12 and CDK13 inhibitors. Nature Chemical Biology 12, 876-884 (2016)). S-2 (10 mg, 0.031 mmol, 1.0 eq) and TSTU (12 mg, 0.040 mmol, 1.3 eq) was dissolved in DMF (1 mL). Then DIPEA (15 μL, 0.093 mmol, 3.0 eq) was added and stirred at room temperature for 1 hour. After the formation of succinate, S-3 (18 mg, 0.047 mmol, 1.5 eq) was added and stirred overnight. Upon completion, the reaction was concentrated, and the resulting residue was purified by flash chromatography to provide MS-DTB as a colorless oil (11.2 mg, 0.016 mmol, 52%).

¹H NMR (500 MHz, CD₃OD) δ 6.80 (s, 2H), 3.85-3.78 (m, 3H), 3.77-3.68 (m, 4H), 3.67-3.60 (m, 9H), 3.58 (t, J=6.0 Hz, 2H), 3.55 (t, J=5.5 Hz, 3H), 3.47-3.39 (m, 2H), 3.38-3.35 (m, 2H), 2.45 (t, J=7.0 Hz, 3H), 2.22 (t, J=7.5 Hz, 3H), 1.63 (p, J=7.5 Hz, 3H), 1.53-1.48 (m, 2H), 1.47-1.35 (m, 3H), 1.11 (d, J=6.0 Hz, 3H).

¹³C NMR (126 MHz, CD₃OD) δ 176.37, 172.85, 172.22, 169.06, 166.23, 135.51, 79.51, 71.61, 71.39, 71.31, 70.62, 70.39, 66.09, 57.43, 52.73, 40.66, 40.40, 39.64, 36.94, 35.78, 35.40, 30.76, 30.24, 27.19, 26.87, 15.68.

HRMS (ESI+) m/z calcd for C₂₈H₄₇N₆O₁₂S⁺ [M+H]⁺: 691.2967, found 691.2956.

### Synthesis of maleimide-sulfonate-dibenzocyclooctyne-DTB (MSD-DTB)

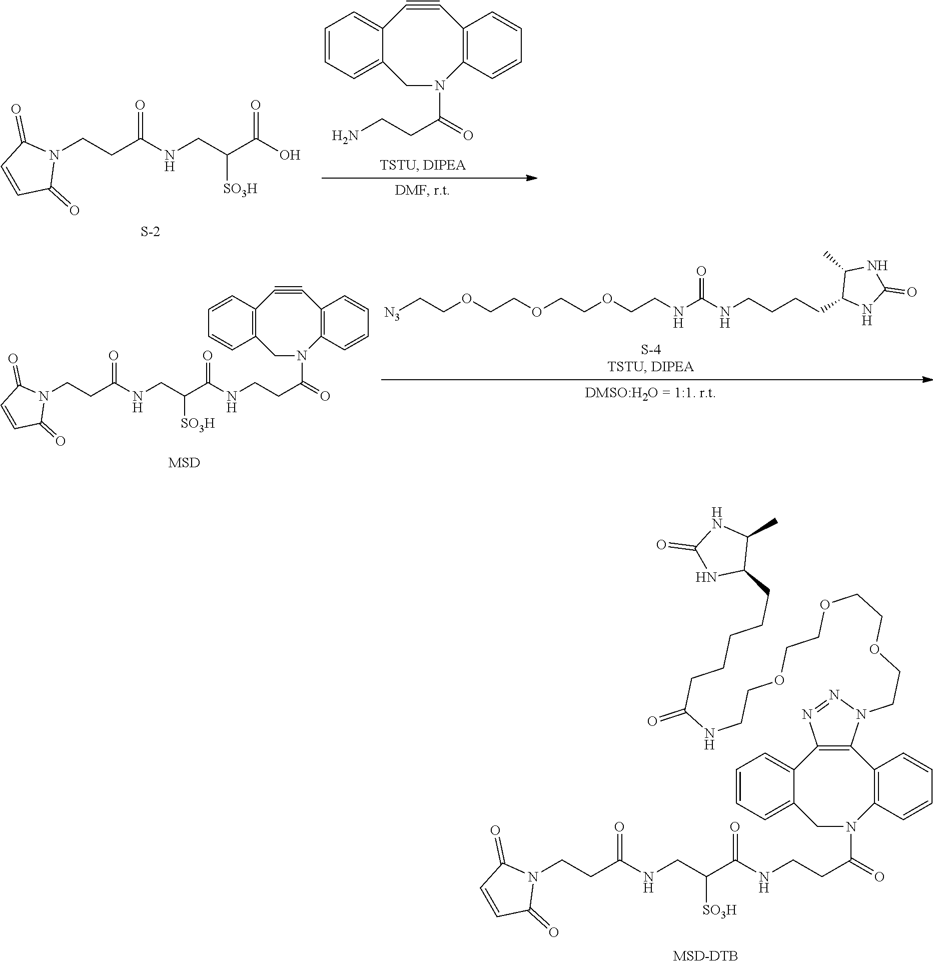

Step 1: S-2 (40 mg, 0.125 mmol, 1.0 eq) and TSTU (49 mg, 0.163 mmol, 1.3 eq) was dissolved in DMF (1 mL). Then DIPEA (149 μL, 0.90 mmol, 3.0 eq) was added and stirred for 1 h at room temperature. After the formation of succinate, dibenzocyclooctyne-amine (52 mg, 0.188 mmol, 1.5 eq) was added and stirred overnight. Upon completion, the reaction was concentrated, and the resulting residue was purified by flash chromatography to provide MSD as a white powder (67 mg, 0.116 mmol, 71%).

¹H NMR (500 MHz, CD₃OD) δ 7.82-7.24 (m, 8H), 6.79-6.78 (m, 2H), 5.15 (dd, J=14.0, 4.5 Hz, 1H), 3.77-3.56 (m, 6H), 3.43-3.37 (m, 1H), 3.28-3.24 (m, 1H), 2.65-2.57 (m, 1H), 2.44-2.36 (m, 2H), 2.07-1.98 (m, 1H).

Step 2: S-4 was prepared as reported previously (Osuna Gálvez, A. & Bode, J. W. Traceless Templated Amide-Forming Ligations. Journal of the American Chemical Society 141, 8721-8726 (2019)). MSD (30 mg, 0.052 mmol, 1.0 eq) and S-4 (22 mg, 0.052 mmol, 1.0 eq) was dissolved in DMSO:H₂O (1:1, 1 mL) and stirred at room temperature for 1 hour. Upon completion, the reaction was concentrated, and the resulting residue was purified by flash chromatography to provide MSD-DTB as a white powder (29.6 mg, 0.030 mmol, 58%).

¹H NMR (500 MHz, CD₃OD) δ 7.72-7.25 (m, 8H), 6.82-6.80 (m, 2H), 6.02-5.96 (m, 1H), 4.71-4.46 (m, 3H), 4.19-4.17 (m, 1H), 3.93-3.89 (m, 1H), 3.82-3.42 (m, 1H), 3.34-3.33 (m, 1H), 3.29-3.22 (m, 2H), 2.49-2.38 (m, 2H), 2.20-2.05 (m, 3H), 1.77-1.70 (m, 1H), 1.64-1.56 (m, 2H), 1.49-1.44 (m, 2H), 1.36-1.28 (m, 4H), 1.09-1.07 (m, 3H).

¹³C NMR (126 MHz, CD₃OD) δ 176.28, 176.23, 173.16, 172.58, 172.29, 166.09, 145.78, 144.19, 142.48, 141.45, 136.96, 135.57, 133.51, 132.83, 132.67, 132.24, 130.99, 130.65, 130.55, 129.89, 129.75, 128.83, 126.06, 71.78, 71.59, 71.53, 71.51, 71.45, 71.25, 70.59, 70.56, 70.52, 57.38, 52.70, 49.88, 40.36, 40.29, 36.92, 36.80, 35.80, 35.39, 30.75, 30.20, 27.16, 26.83, 15.73.

HRMS (ESI+) m/z calcd for C₄₆H₆₁N₁₀O₁₃S⁺ [M+H]⁺: 993.4135, found 993.4123.

### Synthesis of iodoacetamide-PEG-desthiobiotin (IA-DTB) and chloroacetamide-PEG-desthiobiotin (CA-DTB)

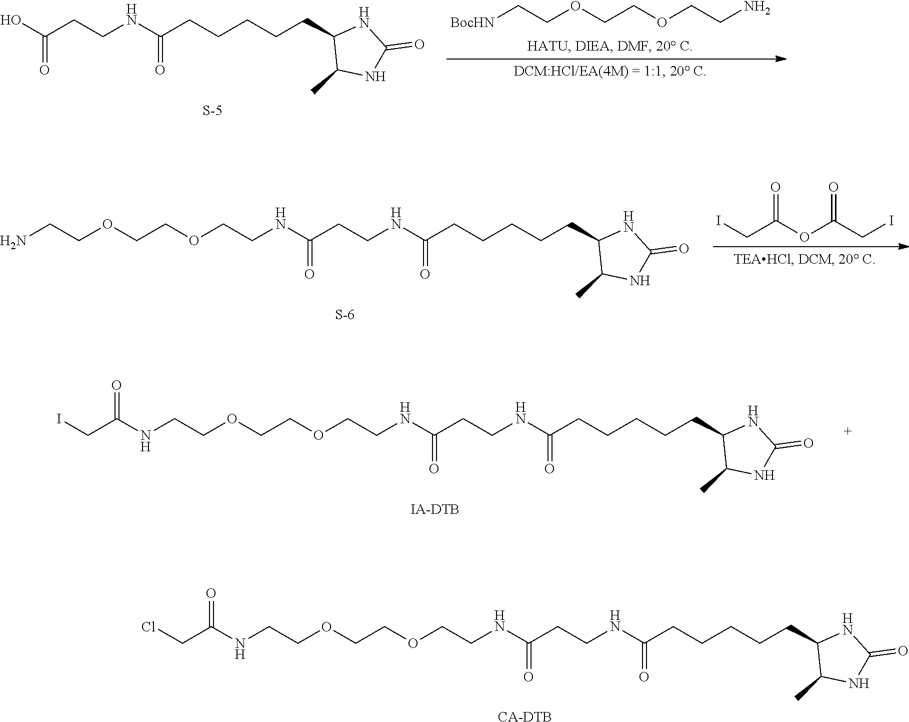

S-5, synthesized by previously reported method (Skander, M. et al. Artificial Metalloenzymes: (Strept) avidin as Host for Enantioselective Hydrogenation by Achiral Biotinylated Rhodium-Diphosphine Complexes. Journal of the American Chemical Society 126, 14411-14418 (2004)), was coupled with N-Boc-2,2′-(ethylenedioxy) diethylamine which followed by deprotection of tert-Butyloxycarbonyl to give S-6. In presence of iodoacetic anhydride and triethylamine hydrochloride, S-6 was stirred in DCM at room temperature for 2 days and finally gave mixture of IA-DTB (10.2 mg, 0.017 mmol, 51%) and CA-DTB (6.6 mg, 0.013 mmol, 33%).

IA-DTB: ¹H NMR (500 MHz, CDCl₃) δ 7.43 (s, 1H), 7.02 (s, 1H), 6.60 (s, 1H), 3.89-3.84 (m, 1H), 3.77-3.70 (m, 3H), 3.63-3.47 (m, 14H), 2.50 (t, J=4.0 Hz, 2H), 2.21 (t, J=8.0 Hz, 2H), 1.68-1.65 (m, 2H), 1.46-1.32 (m, 6H), 1.15 (d, J=8.0 Hz, 3H).

HRMS (ESI+) m/z calcd for C₂₁H₃₉IN₅O₆⁺ [M+H]⁺: 584.1940, found 584.1958.

CA-DTB: ¹H NMR (500 MHz, CDCl₃) δ 7.19 (s, 1H), 6.79 (s, 1H), 6.37 (s, 1H), 4.08 (s, 2H), 3.88-3.84 (m, 1H), 3.73-3.70 (m, 1H), 3.64-3.60 (m, 6H), 3.58-3.52 (m, 6H), 3.48-3.44 (m, 2H), 2.46 (t, J=4.0 Hz, 2H), 2.18 (t, J=8.0 Hz, 2H), 1.67-1.64 (m, 2H), 1.48-1.26 (m, 6H), 1.15 (d, J=4.0 Hz, 3H).

HRMS (ESI+) m/z calcd for C₂₁H₃₉ClN₅O₆+ [M+H]⁺: 492.2583, found 492.2597.

### Synthesis of iodoacetamide-carboxylate-PEG-desthiobiotin (IA-DTB—COOH)

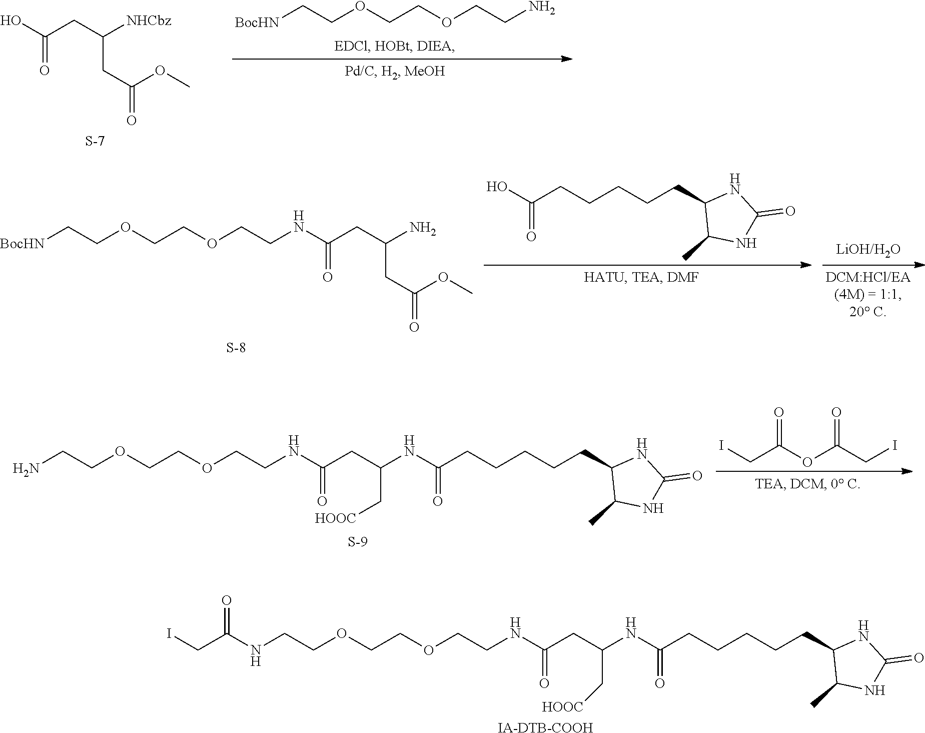

S-7, synthesized by previously reported method (Kadowaki, T., Kainuma, R., Kato, S. & Konno, H. Synthesis and Configuration Confirmation of the ATHOD Fatty Amino Acid Residue in the Burkholdines. Journal of Natural Products 85, 2052-2061 (2022)), was condensation with N-Boc-2,2′-(ethylenedioxy) diethylamine which then underwent deprotection of N-carbobenzyloxy group to give S-8. Intermediate S-8 went through sequentially coupling with D-Desthiobiotin and two steps of hydrolysis provided S-9. By using similar procedure with IA-DTB, IA-DTB-COOH was purified by pre-HPLC (12.7 mg, 0.020 mmol, 87%).

¹H NMR (500 MHz, CDCl₃) δ 7.70 (s, 1H), 7.22-7.15 (m, 1H), 6.00-5.84 (s, 1H), 5.32-5.09 (m, 1H), 4.63-4.39 (m, 1H), 3.66-3.26 (m, 12H), 2.44-2.35 (m, 2H), 2.13-2.02 (m, 2H), 1.47-1.23 (m, 11H), 0.96 (d, J=8.0 Hz, 3H).

¹³C NMR (125 MHz, CD₃OD) δ 177.58, 175.54, 172.90, 171.47, 166.21, 71.39, 71.35, 71.31, 70.55, 70.21, 57.41, 52.73, 52.71, 40.99, 40.33, 37.02, 34.78, 30.71, 30.18, 27.16, 27.14, 26.76, 25.91.

HRMS (ESI+) m/z calcd for C₂₃H₄₁IN₅O₈⁺ [M+H]⁺: 642.1994, found 642.1975.

### Synthesis of desthiobiotin iodoacetamide (DBIA)

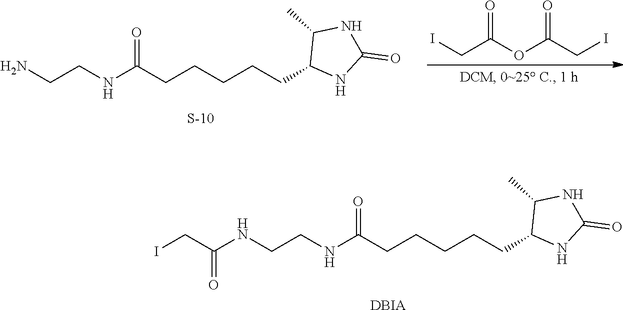

S-10, synthesized by previously reported method (Liu, W. et al. Identification of a Covalent Importin-5 Inhibitor, Goyazensolide, from a Collective Synthesis of Furanoheliangolides. ACS Central Science 7, 954-962 (2021)), was reacted with iodoacetic anhydride in DCM to afford DBIA (50.0 mg, 0.119 mmol, 63%).

¹H NMR (500 MHz, CDCl₃) δ 8.26 (s, 1H), 7.78 (s, 1H), 6.29 (s, 1H), 6.11 (s, 1H), 3.63-3.57 (m, 3H), 3.49-3.46 (m, 1H), 3.07 (s, 4H), 2.04 (t, J=8.0 Hz, 2H), 1.51-1.44 (m, 2H), 1.35-1.28 (m, 6H), 1.27-1.13 (m, 2H), 0.96 (d, J=4.0 Hz, 3H).

HRMS (ESI+) m/z calcd for C₁₄H₂₆IN₄O₃⁺ [M+H]⁺: 425.1044, found 425.1029.

### Synthesis of M-Fc-III-4C

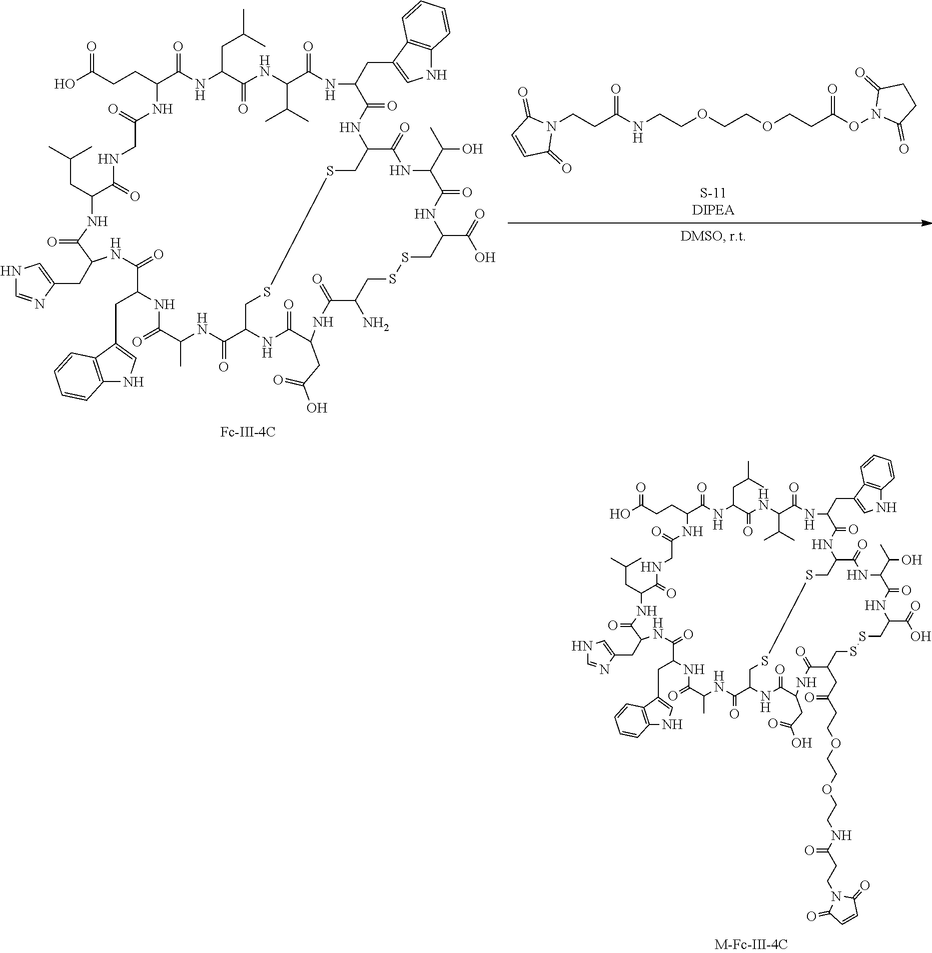

The Fc-III-4C peptide (purchased from GenScript) and Malcimide-PBG2-succinimidyl ester were dissolved in DMSO. Then DIPEA was added and stirred at room temperature for 12 hours. The reaction was purified by HPLC to provide M-Fc-III-4C as white solid. The mass peak of M-Fc-III-4C was shown at m/z 1023.65 in doubly charged state.

What is claimed is:

1. A cell impermeable probe comprising a cysteine-reactive group selected from iodoacetamide, chloroacetamide, and maleimide linked to desthiobiotin by a linker comprising a carboxyl and/or sulfonate functional group to impart cell impermeability.

2. The cell impermeable probe of claim 1, wherein said probe is selected from maleimide-sulfonate-dibenzocyclooctyne-desthiobiotin (MSD-DTB) and maleimide-sulfonate-desthiobiotin (MS-DTB).

3. The cell impermeable probe of claim 1, wherein said maleimide is hydrolyzed or non-hydrolyzed.

4. The cell impermeable probe of claim 1, wherein said probe has a structure selected from

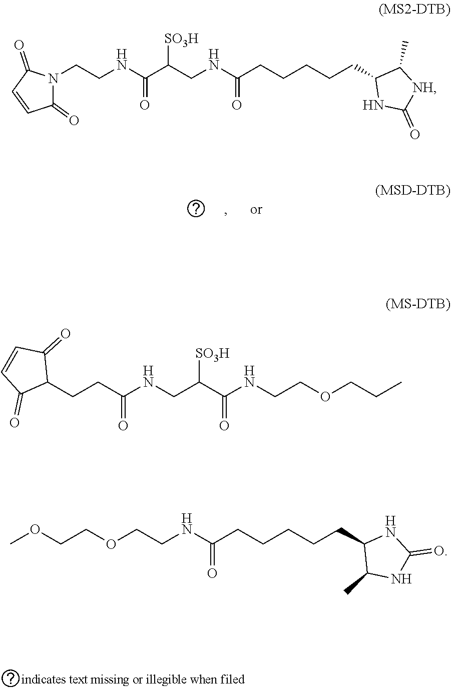

5. A composition, kit, or system, comprising the probe of claim 1 and a streptavidin-bound detectable label.

6. The composition, kit, or system of claim 4, where the detectable label is a fluorophore.

7. The composition, kit, or system of claim 5, where the fluorophore is fluorescein isothiocyanate (FITC).

8. The composition, kit, or system of claim 4, further comprising one or more additional components selected from a buffer, a cytokine, a test compound, and a detection reagent.

9. A method of detecting MHC-I-associated peptides, comprising:
a) contacting a sample comprising MHC-I with a probe of claim 1 under conditions such that said cysteine-reactive group binds to extracellularly-displayed cysteine-containing peptides bound to MHC-1; and
b) detecting said peptides bound to said cysteine-reactive group.

10. The method of claim 9, wherein said detecting comprises contacting the sample with a streptavidin-bound detectable label that binds to said desthiobiotin.

11. The method of claim 10, further comprising detecting the detectable label.

12. The method of claim 11, further comprising isolating the MHC-I-associated peptides and/or cells displaying the MHC-I-associated peptides.

13. The method of claim 9, wherein said peptides are displayed on single-chain trimers (SCTs) comprising covalently linked single chains of MHC-I, β2-microglobulin, and cysteine-containing peptides.

14. The method of claim 12, further comprising identifying the MHC-I-associated peptides.

15. The method of claim 9, further comprising contacting said sample with a test compound; and assaying the effect of the test compound on binding of said probe to cysteines on said peptide.

16. The method of claim 15, wherein said test compound is a bispecific T cell engager (BiTEs).

17. The method of claim 15, wherein said test compound induces antibody-dependent cellular phagocytosis (ADCP).

18. The method of claim 17, wherein said test compound comprises maleimide moiety bound to an Fc-binding cyclic peptide.

19. The method of claim 18, wherein said test compound is

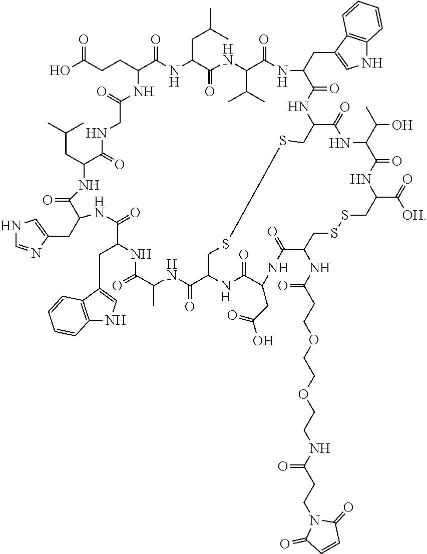 [SMILES: CC(C)CC1NC(=O)C(Cc2c[nH]cn2)NC(=O)C(Cc2c[nH]c3ccccc23)NC(=O)C(C)NC(=O)C2CSSCC(NC(=O)C(Cc3c[nH]c4ccccc34)NC(=O)C(C(C)C)NC(=O)C(CC(C)C)NC(=O)C(CCC(=O)O)NC(=O)CNC1=O)C(=O)NC(C(C)O)C(=O)NC(C(=O)O)CSSCC(NC(=O)CCOCCOCCNC(=O)CCN1C(=O)C=CC1=O)C(=O)NC(CC(=O)O)C(=O)N2]

20. The method of claim 9, wherein the sample is amniotic fluid, ascites, bile, breast milk, breast milk colostrum, bronchoalveolar lavage fluid, cerebrospinal fluid, dialysate, eye aqueous humor, eye vitreous humor, feces, paracentesis, pericardial fluid, peritoneal, blood plasma, pleural, semen, blood serum, synovial fluid, tears, thoracentesis, blood, saliva, gargle, or urine.
